(19) United States
(12) Patent Application Publication
(10) Pub. No.: US 2014/0283216 A1
(43) Pub. Date: Sep. 18, 2014

(54) COMPOSITIONS AND METHODS OF USE OF ACC OXIDASE POLYNUCLEOTIDES AND POLYPEPTIDES

(71) Applicant: PIONEER HI BRED INTERNATIONAL INC, Johnston, IA (US)

(72) Inventors: Xiaoming Bao, Beijing (CN); Jeffrey Habben, Urbandale, IA (US); Sabrina Humbert, Johnston, IA (US)

(73) Assignee: PIONEER HI BRED INTERNATIONAL INC, Johnston, IA (US)

(21) Appl. No.: 14/210,711

(22) Filed: Mar. 14, 2014

Related U.S. Application Data
(60) Provisional application No. 61/792,820, filed on Mar. 15, 2013.

(51) Int. Cl. C12N 15/113

(52) U.S. Cl. CPC C12N 15/1137
USPC 800/285; 800/320.1; 435/412; 536/24.5; 435/320.1; 435/6.18

(57) ABSTRACT

Compositions and methods reduce the expression of endogenous ACC oxidase genes to improve an agronomic characteristic of a crop plant, which may be maize. Yield increase and drought tolerance due to reduction in the endogenous ACC oxidase levels are observed. ACC oxidase genes are identified in maize, rice, and Arabidopsis genomes.

### CROSS REFERENCE

This utility application claims the benefit of U.S. Provisional Application No. 61/792,820, filed Mar. 15, 2013 which is incorporated herein by reference.

### BACKGROUND

Abiotic stress is the primary cause of crop loss worldwide, causing average yield losses more than 50% for major crops (Boyer, (1982) Science 218:443-448; Bray, et al., (2000) In Biochemistry and Molecular Biology of Plants, edited by Buchannan, et al., Amer. Soc. Plant Biol., pp. 1158-1249). Exposure of plants to a water-limiting environment during various developmental stages appears to activate various physiological and developmental changes. Thus there is a need to understand and manipulate biochemical and molecular mechanisms contributing to drought stress tolerance.

Ethylene (C2H4) is a gaseous plant hormone that affects myriad developmental processes and fitness responses in plants, such as germination, flower and leaf senescence, fruit ripening, leaf abscission, root nodulation, programmed cell death and responsiveness to stress and pathogen attack. Ethylene governs diverse processes in plants, and these effects are sometimes affected by the action of other plant hormones, other physiological signals and the environment, both biotic and abiotic.

Ethylene is generated from methionine by a biosynthetic pathway involving the conversion of S-adenosyl-L-methionine (SAM or Ado Met) to the cyclic amino acid 1-aminocyclopropane-1-carboxylic acid (ACC) which is facilitated by ACC synthase. Sulphur is conserved in the process by recycling 5′-methylthioadenosine.

ACC synthase is an aminotransferase which catalyzes the rate-limiting step in the formation of ethylene by converting S-adenosylmethionine to ACC. Typically, the enzyme requires pyridoxal phosphate as a cofactor.

The enzyme 1-aminocyclopropane-1-carboxylic acid oxidase (ACO or ACC oxidase) catalyzes the final step of ethylene biosynthesis which converts ACC and O₂ to ethylene, CO₂, cyanide (HCN) and two H₂O. The ACO enzyme is stereospecific and uses cofactors, e.g., Fe⁺², O₂, ascorbate, etc. Activity of ACO can be inhibited by anoxia and cobalt ions.

### SUMMARY

The disclosure provides methods and compositions for modulating yield, drought tolerance and/or nitrogen utilization efficiency in plants as well as modulating (e.g., reducing) ethylene production in plants. This disclosure provides compositions and methods for down-regulating the level and/or activity of 1-aminocyclopropane-1-carboxylic acid oxidase (ACO or ACC oxidase) in plants.

In certain embodiments are provided methods for modulating the expression of ACO polynucleotides or polypeptides in plants, including the development and deployment of specific RNAi constructs to create plants with improved yield and/or improved abiotic stress tolerance, which may include improved drought tolerance, improved density tolerance, and/or improved NUE (nitrogen utilization efficiency).

A method of improving abiotic stress tolerance in a crop plant, the method includes reducing the expression of an ACC oxidase gene in the crop plant and growing the crop plant in a plant growing environment, wherein the crop plant is exposed to an abiotic stress.

A method of improving drought tolerance in a crop plant, the method includes reducing the expression of an ACC oxidase gene in the crop plant and growing the crop plant in a plant growing environment, wherein the crop plant is exposed to drought stress. In an embodiment, the ACC oxidase gene that is down regulated includes a polynucleotide encoding a polypeptide selected from the group consisting of SEQ ID NOS: 21-30, 59, 61, 63, 65, 67, 69 and 71 or an amino acid sequence that is at least 95% identical to the polypeptide thereof. In an embodiment, the ACC oxidase gene that is down regulated comprises a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

In an embodiment, the ACC oxidase gene is down regulated by a RNA-interference construct that includes a nucleic acid element that targets an endogenous mRNA sequence transcribed a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

In an embodiment, the ACC oxidase gene includes a polynucleotide selected from the group consisting of SEQ ID NOS: 1-20, 31-40, 58, 60, 62, 64, 66, 68 and 70 or a nucleotide sequence that is at least 95% identical to the polynucleotide thereof and wherein the ACC oxidase gene is down regulated by a genetic modification.

An abiotic stress tolerant transgenic maize plant comprising in its genome a recombinant nucleic acid that down regulates the expression of an endogenous ACO gene, wherein the ACO gene includes a polynucleotide that encodes a polypeptide selected from the group consisting of SEQ ID NOS: 21-30. The abiotic stress is drought or low nitrogen. In an embodiment, the recombinant nucleic acid down regulates the expression of ACO2, ACO5, and ACO6. In an embodiment, the recombinant nucleic acid sequences comprise a polynucleotide sequence selected from the group consisting of SEQ ID NOS: 41-43.

In an embodiment, in the maize plant, the ACO2 is suppressed by the recombinant nucleic acid sequences comprising SEQ ID NO: 41, the ACO5 is suppressed by the recombinant nucleic acid sequences comprising SEQ ID NO: 42 and the ACO6 is suppressed by the recombinant nucleic acid sequences comprising SEQ ID NO: 43. In an embodiment, the maize plant includes in its genome wherein the nucleic acid simultaneously down regulates the expression of ACO2, ACO5 and ACO6.

A plant cell produced from the maize plant described herein is disclosed.

A seed produced from the maize plant described herein is disclosed.

A method of increasing grain yield of a crop plant under drought conditions, the method includes reducing the levels of ethylene in the crop plant, wherein the reduction in ethylene levels are not accompanied by a reduction in ACC levels within the crop plant and growing the crop plant in a crop growing condition, wherein the crop plant is exposed to drought stress and thereby increasing the grain yield of the crop plant. In an embodiment, the crop plant is maize. In an embodiment, the ethylene levels are reduced by the down regulation of a gene encoding an ACC oxidase. In an embodiment, the ACC oxidase gene that is down regulated includes a polynucleotide encoding a polypeptide selected from the group consisting of SEQ ID NOS: 21-30, 59, 61, 63, 65, 67, 69 and 71 or an amino acid sequence that is at least 95% identical to the polypeptide thereof. In an embodiment, the ACC oxidase gene that is down regulated includes a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

A gene down regulation construct comprising an isolated nucleic acid that is transcribed in to a plurality of interfering RNA transcripts, wherein the interfering RNA transcripts reduce the expression of a plurality of polynucleotide sequences that encode a plurality of polypeptides selected from the group consisting of SEQ ID NOS: 21-30, 59, 61, 63, 65, 67, 69 and 71 or an amino acid sequence that is at least 95% identical to the polypeptide thereof. In an embodiment, the construct is a hairpin construct.

A vector that includes the recombinant nucleic acids and constructs described herein are disclosed.

A method of down regulation of an endogenous ACC oxidase gene in a maize plant, the method includes expressing a recombinant nucleic acid construct that reduces the expression of the endogenous ACC oxidase selected from the group consisting of SEQ ID NOS: 1-20 or an allelic variant of the sequences thereof. In an embodiment, the expression of the endogenous ACC oxidase gene is reduced by a recombinant construct comprising a polynucleotide sequence selected from the group consisting of SEQ ID NOS: 41-43. In an embodiment, the ACC oxidase gene that is being down regulated is selected from the group consisting of SEQ ID NOS: 3-6, 11-12, 32-33, 36 and 39 or a nucleotide sequence that is an allelic variant of SEQ ID NOS: 3-6, 11-12, 32-33, 36 and 39. In an embodiment, the ACC oxidase gene is ACO2. In an embodiment, the ACC oxidase gene includes a polynucleotide encoding a polypeptide selected from the group consisting of SEQ ID NOS: 22 and 23. In an embodiment, the crop plant is monocot.

A method of selecting a maize plant from a population of maize plants for increased drought tolerance, the method includes screening a population of plants for a reduced expression of an ACO gene selected from the group consisting of SEQ ID NOS: 1-20 or an allelic variant of the sequences thereof. In an embodiment, the maize population is an inbred population.

### BRIEF DESCRIPTION OF THE DRAWINGS

FIG. 1. Phylogenetic relationship of ACC oxidase genes based on the encoded proteins.

FIG. 2 shows that the RNAi construct targeting ACO2 effectively reduced endogenous ACO2 transcript levels relative to the control. Data points “E1” through “E15” refer to Event 1 through Event 15. Data point “Cntrl” refers to Control.

FIG. 3 shows that endogenous ACO2, ACO5 and ACO6 expression was reduced to varying degrees by expression of an RNAi construct targeting ACO2, ACO5 and ACO6 as described in Example 3. Data points “E1” through “E15” refer to Event 1 through Event 15. Data point “Cntrl” refers to Control.

### BRIEF DESCRIPTION OF THE SEQUENCES

TABLE 1
Description of sequences and the listing.

| SEQ ID | Name |
|---|---|
| 1 | ZmACO1_transcribed |
| 2 | ZmACO1_cds |
| 3 | ZmACO2-1_transcribed |
| 4 | ZmACO2-1_cDNA |
| 5 | ZmACO2-2_transcribed |
| 6 | ZmACO2-2_cDNA |
| 7 | ZmACO3_transcribed |
| 8 | ZmACO3_cDNA |
| 9 | ZmACO4_transcribed |
| 10 | ZmACO4_cDNA |
| 11 | ZmACO5_transcribed |
| 12 | ZmACO5_cDNA |
| 13 | ZmACO8-1_transcribed |
| 14 | ZmACO8-1_cDNA |
| 15 | ZmACO8-2_transcribed |
| 16 | ZmACO8-2_cDNA |
| 17 | ZmACO6_transcribed |
| 18 | ZmACO6_cDNA |
| 19 | ZmACO9_transcribed |
| 20 | ZmACO9_cDNA |
| 21 | ZmACO1_aa |
| 22 | ZmACO2-1_aa |
| 23 | ZmACO2-2_aa |
| 24 | ZmACO3_aa |
| 25 | ZmACO4_aa |
| 26 | ZmACO5_aa |
| 27 | ZmACO8-1_aa |
| 28 | ZmACO8-2_aa |
| 29 | ZmACO6_aa |
| 30 | ZmACO9_aa |
| 31 | ZmACO1_genomic |
| 32 | ZmACO2-1_genomic |
| 33 | ZmACO2-2_genomic |
| 34 | ZmACO3_genomic |
| 35 | ZmACO4_genomic |
| 36 | ZmACO5_genomic |
| 37 | ZmACO8-1_genomic |
| 38 | ZmACO8-2_genomic |
| 39 | ZmACO6_genomic |
| 40 | ZmACO9_genomic |
| 41 | Construct_1(ACO2) |
| 42 | Construct_2(ACO5) |
| 43 | Construct_3(ACO6) |
| 44 | AT1G03400.1_DNA |
| 45 | AT1G03400.1_aa |
| 46 | AT1G62380.1_DNA_ACO2 |
| 47 | AT1G62380.1_aa_ACO2 |
| 48 | AT2G19590.1_DNA_ACO1 |
| 49 | AT2G19590.1_aa_ACO1 |
| 50 | AT2G25450.1_DNA |
| 51 | AT2G25450.1_aa |
| 52 | AT5G43440.1_DNA |
| 53 | AT5G43440.1_aa |
| 54 | AT5G43440.2_DNA |
| 55 | AT5G43440.2_aa |
| 56 | AT5G43450.1_DNA |
| 57 | AT5G43450.1_aa |
| 58 | Os02g0771600_ACO2_DNA |
| 59 | Os02g0771600_ACO2_aa |
| 60 | Os09g0451000_ACO1_DNA |
| 61 | Os09g0451000_ACO1_aa |
| 62 | Os09g0451400_DNA |
| 63 | Os09g0451400_aa |
| 64 | Os01g0580500_DNA |
| 65 | Os01g0580500_aa |
| 66 | Os11g0186900_DNA |
| 67 | Os11g0186900_aa |
| 68 | Os05g0149400_DNA |
| 69 | Os05g0149400_aa |
| 70 | Os05g0149300_DNA |
| 71 | Os05g0149300_aa |

A sequence listing is provided herewith in electronic medium. The contents of the sequence listing are hereby incorporated by reference in compliance with 37 CFR 1.52(e)

### DETAILED DESCRIPTION

Regulation of ZmACO provides methods to manipulate ACC for reducing ethylene levels and increasing drought stress tolerance. Regulation of ZmACO may be used in combination with other methods, such as manipulation of ACS expression, for reducing ethylene levels and increasing drought tolerance. Specific tissues may be targeted for regulation of ACO and/or ACS. ACC is highly mobile in the plant and several options can be implemented to regulate ACC levels including for example, ACO down regulation or ACS down regulation or a combination of both. ZmACO RNAi constructs are efficacious because endogenous ZmACO transcript levels are relatively high.

In certain embodiments, the present disclosure is directed to a transgenic plant or plant cell containing a polynucleotide comprising a down-regulation construct. In certain embodiments, a plant cell of the disclosure is from a dicot or monocot. Preferred plants containing the polynucleotides include, but are not limited to, maize, soybean, sunflower, sorghum, canola, wheat, alfalfa, cotton, rice, barley, tomato and millet. In certain embodiments, the transgenic plant is a maize plant or plant cell. A transgenic seed comprising a transgenic down-regulation construct as described herein is an embodiment. In one embodiment, the plant cell is in a hybrid or inbred plant comprising improved drought tolerance and/or an improved nitrogen use efficiency and/or improved yield, relative to a control. Plants may comprise a combination of such phenotypes. A plant regenerated from a plant cell of the disclosure is also a feature.

Certain embodiments have improved drought tolerance as compared to a control plant. The improved drought tolerance of a plant of the disclosure may reflect physiological aspects such as, but not limited to, (a) a reduction in the production of at least one ACO-encoding mRNA; (b) a reduction in the production of an ACO; (c) a reduction in the production of ACC; (d) a reduction in the production of ethylene; (e) an increase in plant height or (f) any combination of (a)-(e), compared to a corresponding control plant. Plants exhibiting improved drought tolerance may also exhibit one or more additional abiotic stress tolerance phenotyopes, such as improved nitrogen utilization efficiency or increased density tolerance.

A method of improving abiotic stress tolerance in a crop plant, the method includes reducing the expression of an ACC oxidase gene in the crop plant and growing the crop plant in a plant growing environment, wherein the crop plant is exposed to an abiotic stress. Abiotic stresses can include nutrient stress, water stress, drought, cold, frost, salt, heat, and nitrogen stress.

A method of improving drought tolerance in a crop plant, the method includes reducing the expression of an ACC oxidase gene in the crop plant and growing the crop plant in a plant growing environment, wherein the crop plant is exposed to drought stress or grown in conditions that are likely to result in water stress. In an embodiment, the ACC oxidase gene that is down regulated includes a polynucleotide encoding a polypeptide selected from the group consisting of SEQ ID NOS: 21-30, 59, 61, 63, 65, 67, 69 and 71 or an amino acid sequence that is at least 95% identical to the polypeptide thereof. In an embodiment, the ACC oxidase gene that is down regulated comprises a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

In an embodiment, the ACC oxidase gene is down regulated by a RNA-interference construct that includes a nucleic acid element that targets an endogenous mRNA sequence transcribed a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

In an embodiment, the ACC oxidase gene includes a polynucleotide selected from the group consisting of SEQ ID NOS: 1-20, 31-40, 58, 60, 62, 64, 66, 68 and 70 or a nucleotide sequence that is at least 95% identical to the polynucleotide thereof and wherein the ACC oxidase gene is down regulated by a genetic modification.

An abiotic stress tolerant transgenic maize plant comprising in its genome a recombinant nucleic acid that down regulates the expression of an endogenous ACO gene, wherein the ACO gene includes a polynucleotide that encodes a polypeptide selected from the group consisting of SEQ ID NOS: 21-30. The abiotic stress is drought or low nitrogen. In an embodiment, the recombinant nucleic acid down regulates the expression of ACO2, ACO5, and ACO6. In an embodiment, the recombinant nucleic acid sequences comprise a polynucleotide sequence selected from the group consisting of SEQ ID NOS: 41-43.

In an embodiment, in the maize plant, the ACO2 is suppressed by the recombinant nucleic acid sequences comprising SEQ ID NO: 41, the ACO5 is suppressed by the recombinant nucleic acid sequences comprising SEQ ID NO: 42 and the ACO6 is suppressed by the recombinant nucleic acid sequences comprising SEQ ID NO: 43. In an embodiment, the maize plant includes in its genome wherein the nucleic acid simultaneously down regulates the expression of ACO2, ACO5 and ACO6.

A plant cell produced from the maize plant described herein is disclosed.

A seed produced from the maize plant described herein is disclosed.

A method of increasing grain yield of a crop plant under drought conditions, the method includes reducing the levels of ethylene in the crop plant, wherein the reduction in ethylene levels are not accompanied by a reduction in ACC levels within the crop plant and growing the crop plant in a crop growing condition, wherein the crop plant is exposed to drought stress and thereby increasing the grain yield of the crop plant. In an embodiment, the crop plant is maize. In an embodiment, the ethylene levels are reduced by the down regulation of a gene encoding an ACC oxidase. In an embodiment, the ACC oxidase gene that is down regulated includes a polynucleotide encoding a polypeptide selected from the group consisting of SEQ ID NOS: 21-30, 59, 61, 63, 65, 67, 69 and 71 or an amino acid sequence that is at least 95% identical to the polypeptide thereof. In an embodiment, the ACC oxidase gene that is down regulated includes a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

A gene down regulation construct comprising an isolated nucleic acid that is transcribed in to a plurality of interfering RNA transcripts, wherein the interfering RNA transcripts reduce the expression of a plurality of polynucleotide sequences that encode a plurality of polypeptides selected from the group consisting of SEQ ID NOS: 21-30, 59, 61, 63, 65, 67, 69 and 71 or an amino acid sequence that is at least 95% identical to the polypeptide thereof. In an embodiment, the construct is a hairpin construct.

A vector that includes the recombinant nucleic acids and constructs described herein are disclosed. The vector can be a plant expressible vector or contains a plant expressible regulatory element. Suitable promoters include drought inducible promoters such as Rab17 and Rad29.

A method of down regulation of an endogenous ACC oxidase gene in a maize plant, the method includes expressing a recombinant nucleic acid construct that reduces the expression of the endogenous ACC oxidase selected from the group consisting of SEQ ID NOS: 1-20 or an allelic variant of the sequences thereof. In an embodiment, the expression of the endogenous ACC oxidase gene is reduced by a recombinant construct comprising a polynucleotide sequence selected from the group consisting of SEQ ID NOS: 41-43. In an embodiment, the ACC oxidase gene that is being down regulated is selected from the group consisting of SEQ ID NOS: 3-6, 11-12, 32-33, 36 and 39 or a nucleotide sequence that is an allelic variant of SEQ ID NOS: 3-6, 11-12, 32-33, 36 and 39. Allelic variations can occur in the coding region or the promoter or the intron regions of a gene or a genomic locus. In an embodiment, the ACC oxidase gene is ACO2. In an embodiment, the ACC oxidase gene includes a polynucleotide encoding a polypeptide selected from the group consisting of SEQ ID NOS: 22 and 23. In an embodiment, the crop plant is a monocot crop plant such as maize, rice, sorghum, and wheat. In an embodiment, the dicot crop plants include for example soybean and brassica.

A method of selecting a maize plant from a population of maize plants for increased drought tolerance, the method includes screening a population of plants for a reduced expression of an ACO gene selected from the group consisting of SEQ ID NOS: 1-20 or an allelic variant of the sequences thereof. In an embodiment, the maize population is an inbred population. Such screening also may include sequencing of the genomic locus of the ACO genes disclosed herein. In an embodiment, the screening may include analyzing the mRNA levels or protein levels of ACO.

### Methods for Modulating Drought Tolerance in a Plant

Methods for modulating drought tolerance in plants are also features of the disclosure. The ability to introduce different degrees of drought tolerance into plants offers flexibility in the use of the disclosure: for example, introduction of strong drought tolerance for improved grain-filling or for silage in areas with longer or drier growing seasons, versus the introduction of a moderate drought tolerance for silage in agricultural areas with shorter growing seasons. Modulation of drought tolerance of a plant of the disclosure may reflect one or more of the following: (a) a reduction in the production of at least one ACO-encoding mRNA; (b) a reduction in the production of an ACO; (c) a reduction in the production of ethylene; (d) an increase in plant height or (f) any combination of (a)-(e), compared to a corresponding control plant.

For example, methods include: (a) selecting at least one ACO gene; (b) introducing into a plant a polynucleotide targeting expression of the selected ACO gene; and (c) expressing the polynucleotide, thereby modulating drought tolerance in the plant. Plants produced by such methods are also a feature of the disclosure. The degree of drought tolerance introduced into a plant can be determined by a number of factors, e.g., which ACO gene is selected, whether the introduced polynucleotide is present in a heterozygous or homozygous state, or by the number of members of the ACO gene family which are inactivated, or by a combination of two or more such factors.

Once the desired ACO gene is selected, a polynucleotide targeting expression of the ACO gene is introduced into a plant. In certain embodiments, the polynucleotide is introduced by Agrobacterium-mediated transfer, electroporation, micro-projectile bombardment, homologous recombination or a sexual cross. In certain embodiments, the polynucleotide includes a subsequence of the selected ACO gene in an antisense, sense or RNA silencing or interference configuration. In certain embodiments, more than one ACO gene is selected for targeting. In certain embodiments, a polynucleotide may target more than one ACO gene. In certain embodiments, multiple polynucleotides are used to target the selected ACO genes.

Expression of the polynucleotide targeting the ACO gene can be determined in a number of ways. For example, detection of expression products is performed either qualitatively (presence or absence of one or more products of interest) or quantitatively (by monitoring the level of expression of one or more products of interest). In one embodiment, the expression product is an RNA expression product. The disclosure optionally includes monitoring the expression level of a nucleic acid or polypeptide as noted herein for detection of ACO in a plant or in a population of plants. Monitoring levels of ethylene or ACC can also serve to detect down-regulation of expression or activity of the ACO gene.

By “flowering stress” is meant that water is withheld from plants such that drought stress occurs at or around the time of anthesis.

By “grain fill stress” is meant that water is withheld from plants such that drought stress occurs during the time when seeds are accumulating storage products (carbohydrates, protein and/or oil).

By “rain-fed conditions” is meant that water is neither deliberately withheld nor artificially supplemented.

By “well-watered conditions” is meant that water available to the plant is generally adequate for optimum growth.

Drought stress conditions for maize may be controlled to result in a targeted yield reduction. For example, a 20%, 30%, 40%, 50%, 60%, 70%, or greater reduction in yield of control plants can be accomplished by providing measured amounts of water during specific phases of plant development.

“Drought” refers to a decrease in water availability to a plant that, especially when prolonged or when occurring during critical growth periods, can cause damage to the plant or prevent its successful growth (e.g., limiting plant growth or seed yield).

“Drought tolerance” reflects a plant's ability to survive under drought without exhibiting substantial physiological or physical deterioration, and/or its ability to recover when water is restored following a period of drought.

“Drought tolerance activity” of a polypeptide indicates that over-expression of the polypeptide in a transgenic plant confers increased drought tolerance of the transgenic plant relative to a reference or control plant.

“Increased drought tolerance” of a plant is measured relative to a reference or control plant, and reflects ability of the plant to survive under drought conditions with less physiological or physical deterioration than a reference or control plant grown under similar drought conditions or ability of the plant to recover more substantially and/or more quickly than would a control plant when water is restored following a period of drought.

### Methods for Modulating Density Tolerance in a Plant

In addition to increasing plant tolerance to drought stress, the disclosure also may enable higher density planting of plants of the disclosure, leading to increased yield per acre. In maize, for example, much of the increased yield per acre over the last century has come from increasing tolerance to density, which is a stress to plants. Methods for modulating plant stress response, e.g., increasing tolerance for density, are also a feature of the disclosure. For example, a method of the disclosure can include: (a) selecting at least one ACO gene; (b) introducing into a plant a polynucleotide targeting expression of the selected ACO gene; and (c) expressing the polynucleotide, thereby modulating density tolerance in the plant. Plants produced by such methods are also a feature of the disclosure. When ethylene production is reduced in a plant by regulation of expression of an ACO gene, the plant may have a reduced perception of and/or response to density. Thus, plants of the disclosure can be planted at higher density and produce an increase in yield of seed and/or biomass.

### Methods for Modulating Nitrogen Utilization Efficiency in a Plant

In addition to increasing plant tolerance to drought stress and improving plant density tolerance, the disclosure may also provide greater nitrogen utilization efficiency (NUE). For example, a method of the disclosure can include: (a) selecting at least one ACO gene; (b) introducing into a plant a polynucleotide targeting expression of the selected ACO gene; and (c) expressing the polynucleotide, thereby modulating NUE in the plant. Plants produced by such methods are also a feature of the disclosure. NUE reflects plant ability to uptake, assimilate, and/or otherwise utilize nitrogen.

Plants in which NUE is improved may be more productive than control plants under comparable conditions of ample nitrogen availability and/or may maintain productivity under significantly reduced nitrogen availability. Improved NUE may be reflected in one or more attributes such as increased biomass, increased grain yield, increased harvest index, increased photosynthetic rates and increased tolerance to biotic or abiotic stress. In particular, improving NUE in maize would increase harvestable yield per unit of input nitrogen fertilizer, both in developing nations where access to nitrogen fertilizer is limited and in developed nations where the level of nitrogen use remains high.

### Screening/Characterization of Plants or Plant Cells

Plants can be screened and/or characterized in many ways, e.g. genotypically, biochemically, phenotypically or by any combination of two or more of these methods. For example, plants may be characterized to determine the presence, absence and/or expression level (e.g., amount, modulation, such as a decrease or increase compared to a control cell) of a polynucleotide of the disclosure; the presence, absence, expression and/or enzymatic activity of a polypeptide of the disclosure; and/or modulation of drought tolerance, modulation of nitrogen use efficiency, modulation of density tolerance and/or modulation of ethylene production.

Molecules such as ACC and ethylene can be recovered and assayed from cell extracts. For example, internal concentrations of ACC can be assayed by LC-MS (liquid chromatography-mass spectrometry), in acidic plant extracts as ethylene after decomposition in alkaline hypochlorite solution, etc. The concentration of ethylene can be determined by, e.g., gas chromatography-mass spectroscopy, etc. See, e.g., Nagahama, et al., (1991) J. Gen. Microbiol. 137:2281-2286. For example, ethylene can be measured with a gas chromatograph equipped with, e.g., an alumina based column (such as an HP-PLOT A1203 capillary column (Agilent Technologies, Santa Clara, Calif.) and a flame ionization detector.

Phenotypic analysis includes, e.g., analyzing changes in chemical composition, morphology, or physiological properties of the plant. For example, phenotypic changes can include, but are not limited to, an increase in drought tolerance, an increase in density tolerance, an increase in nitrogen use efficiency and a decrease in ethylene production.

A variety of assays can be used for monitoring drought tolerance and/or NUE. For example, assays include, but are not limited to, visual inspection, monitoring photosynthesis measurements, and measuring levels of chlorophyll, DNA, RNA and/or protein content of, e.g., the leaves, under stress and non-stress conditions.

For example, plants are grown in the field under normal and drought-stress conditions. Under normal conditions, plants are watered with an amount sufficient for optimum growth and yield. For drought-stressed plants, water may be limited for a period starting approximately one week before pollination and continuing through three weeks after pollination. During the period of limited water availability, drought-stressed plants may show visible signs of wilting and leaf rolling. The degree of stress may be calculated as % yield reduction relative to that obtained under well-watered conditions. Transpiration, stomatal conductance and CO₂ assimilation are determined with a portable TPS-1 Photosynthesis System (PP Systems, Amesbury, Mass.). Each leaf on a plant may be measured, e.g. at forty days after pollination. Values typically represent a mean of six determinations.

The term “trait” refers to a physiological, morphological, biochemical or physical characteristics of a plant or particular plant material or cell. In some instances, this characteristics is visible to the human eye, such as seed or plant size, or can be measured by biochemical techniques, such as detecting the protein, starch or oil content of seed or leaves, or by observation of a metabolic or physiological process, e.g. by measuring tolerance to water deprivation or particular salt or sugar or nitrogen concentrations, or by the observation of the expression level of a gene or genes, or by agricultural observations such as osmotic stress tolerance or yield.

“Agronomic characteristics” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop cultivars may be developed to produce higher yield of the vegetative portion of the plant, to be used in food, feed, fiber, and/or biofuel.

Increased leaf size may be of particular interest. Increased leaf biomass can be used to increase production of plant-derived pharmaceutical or industrial products. Increased tiller number may be of particular interest and can be used to increase yield. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits, such as in the forms of greater yield or improved screening.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to a condition or stimulus that would induce expression of the gene of interest; or (e) the subject plant or plant cell itself, under conditions in which the gene of interest is not expressed.

“Genome” as it applies to plant cells encompasses not only chromosomal DNA found within the nucleus, but also organelle DNA found within subcellular components (e.g., mitochondria, plastid) of the cell.

“Plant” includes reference to whole plants, plant organs, plant tissues, seeds and plant cells and progeny of the same. Plant cells include, without limitation, cells from seeds, suspension cultures, embryos, meristematic regions, callus tissues, leaves, roots, shoots, gametophytes, sporophytes, pollen and microspores.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct. A T0 plant is directly recovered from the transformation and regeneration process. Progeny of T0 plants are referred to as T1 (first progeny generation), T2 (second progeny generation), etc.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence” and “nucleic acid fragment” are used interchangeably and refer to a polymer of RNA or DNA that is single- or double-stranded, optionally containing synthetic, non-natural or altered nucleotide bases. Nucleotides (usually found in their 5′-monophosphate form) are referred to by their single-letter designation as follows: “A” for adenylate or deoxyadenylate, “C” for cytidylate or deoxycytidylate and “G” for guanylate or deoxyguanylate for RNA or DNA, respectively; “U” for uridylate; “T” for deoxythymidylate; “R” for purines (A or G); “Y” for pyrimidines (C or T); “K” for G or T; “H” for A or C or T; “I” for inosine and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence” and “protein” are also inclusive of modifications including, but not limited to, glycosylation, lipid attachment and sulfation, gamma-carboxylation of glutamic acid residues, hydroxylation and ADP-ribosylation.

“Messenger RNA (mRNA)” refers to the RNA which has no intron and can be translated into protein by the cell.

“cDNA” refers to a DNA that is complementary to and synthesized from an mRNA template using reverse transcriptase. The cDNA can be single-stranded or converted into the double-stranded form using the Klenow fragment of DNA polymerase I.

“Mature” protein refers to a post-translationally processed polypeptide; i.e., any pre- or pro-peptides present in the primary translation product has been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterogonous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources or regulatory sequences and coding sequences derived from the same source, but arranged in a manner different than that normally found in nature.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and influencing the transcription, RNA processing or stability, or translation of the associated coding sequence. Regulatory sequences may include, but are not limited to, promoters, translation leader sequences, introns and poly-adenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription of genes in plant cells whether or not its origin is from a plant cell.

“Tissue-specific promoter” and “tissue-preferred promoter” may refer to a promoter that is expressed predominantly but not necessarily exclusively in one tissue or organ, but that may also be expressed in one specific cell or cell type.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

An “allele” is one of two or more alternative forms of a gene occupying a given locus on a chromosome. When the alleles present at a given locus on a pair of homologous chromosomes in a diploid plant are the same, that plant is homozygous at that locus. If the alleles present at a given locus on a pair of homologous chromosomes in a diploid plant differ, that plant is heterozygous at that locus. If a transgene is present on one of a pair of homologous chromosomes in a diploid plant, that plant is hemizygous at that locus.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required, or no water, over a period of time, and one can evaluate drought tolerance by observing and measuring differences in physiological and/or physical condition, including (but not limited to) vigor, overall growth, leaf color, or size or growth rate of one or more tissues (e.g. leaf or root). Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-20 days. Acute stress may last 3-15 days. The following variables may be measured during drought stress and well-watered treatments of transgenic plants and relevant control plants:

The variable “% area chg_start chronic-acute 2” is a measure of the percent change in total area determined by remote visible spectrum imaging between the first day of chronic stress and the day of the second acute stress.

The variable “% area chg_start chronic-end chronic” is a measure of the percent change in total area determined by remote visible spectrum imaging between the first day of chronic stress and the last day of chronic stress.

The variable “% area chg_start chronic-harvest” is a measure of the percent change in total area determined by remote visible spectrum imaging between the first day of chronic stress and the day of harvest.

The variable “% area chg_start chronic-recovery 24 h” is a measure of the percent change in total area determined by remote visible spectrum imaging between the first day of chronic stress and 24 h into the recovery (24 h after acute stress 2).

The variable “psii_acute 1” is a measure of Photosystem II (PSII) efficiency at the end of the first acute stress period. It provides an estimate of the efficiency at which light is absorbed by PSII antennae and is directly related to carbon dioxide assimilation within the leaf.

The variable “psii_acute 2” is a measure of Photosystem II (PSII) efficiency at the end of the second acute stress period. It provides an estimate of the efficiency at which light is absorbed by PSII antennae and is directly related to carbon dioxide assimilation within the leaf.

The variable “fv/fm_acute 1” is a measure of the optimum quantum yield (Fv/Fm) at the end of the first acute stress-(variable fluorescence difference between the maximum and minimum fluorescence/maximum fluorescence) The variable “fv/fm_acute 2” is a measure of the optimum quantum yield (Fv/Fm) at the end of the second acute stress-(variable fluorescence difference between the maximum and minimum fluorescence and maximum fluorescence).

The variable “leaf rolling_harvest” is a measure of the ratio of top image to side image on the day of harvest.

The variable “leaf rolling_recovery 24 h” is a measure of the ratio of top image to side image 24 hours (h) into the recovery.

The variable “specific growth rate (SGR)” represents the change in total plant surface area (as measured by LemnaTec Instrument) over a single day (Y (t)=Y0e^{r*t}). Y(t)=Y0e^{r*t} is equivalent to % change in Y/Δt where the individual terms are as follows: Y(t)=Total surface area at t; Y0=Initial total surface area (estimated); r=Specific Growth Rate day⁻¹ and t=Days After Planting (“DAP”).

The variable “shoot dry weight” is a measure of the shoot weight 96 h after being placed into a 104° C. oven.

The variable “shoot fresh weight” is a measure of the shoot weight immediately after being cut from the plant.

Soil plant analyses development (SPAD) value is SPAD reading which is measured by SPAD-502 plus (a chlorophyll meter, made by KONICA MINOLTA). the SPAD value is relative content of leaf chlorophyll and an important indicator of plant health. Many studies indicated that a significant and positive correlation was observed between leaf nitrogen content and SPAD value (Swain and Sandip, (2010) Journal of Agronomy 9(2):38-44) and leaf SPAD value is used as index of nitrogen status diagnosis in crops (Cai, et al., (2010) Acta metallurgica sinica 16(4):866-873).

The SPAD value is measured during low nitrogen treatment.

The Examples below describe some representative protocols and techniques for simulating drought conditions and/or evaluating drought tolerance.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% yield) in field testing under simulated or naturally-occurring drought conditions (e.g., by measuring for substantially equivalent yield under drought conditions compared to non-drought conditions or by measuring for less yield loss under drought conditions compared to yield loss exhibited by a control or reference plant).

Parameters such as gene expression level, water use efficiency, level or activity of an encoded protein and others are typically presented with reference to a control cell or control plant. A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration. One of ordinary skill in the art would readily recognize a suitable control or reference plant to be utilized when assessing or measuring an agronomic characteristics or phenotype of a transgenic plant described herein.

### Use in Breeding Methods

The transformed plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity and plant and ear height is desirable. Traditional plant breeding is an important tool in developing new and improved commercial crops. This disclosure encompasses methods for producing a maize plant by crossing a first parent maize plant with a second parent maize plant wherein one or both of the parent maize plants is a transformed plant displaying a drought tolerance phenotype, a sterility phenotype, a density tolerance phenotype or the like, as described herein.

Plant breeding techniques known in the art and used in a maize plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of maize hybrids in a maize plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular maize plant using transformation techniques can be moved into another line using traditional breeding techniques that are well known in the plant breeding arts. For example, a backcrossing approach is commonly used to move a transgene from a transformed maize plant to an elite inbred line and the resulting progeny would then comprise the transgene(s). Also, if an inbred line was used for the transformation, then the transgenic plants could be crossed to a different inbred in order to produce a transgenic hybrid maize plant. As used herein, “crossing” can refer to a simple X by Y cross or the process of backcrossing, depending on the context.

The development of a maize hybrid in a maize plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process in maize, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Transgenic plants of the present disclosure may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

All references referred to are incorporated herein by reference.

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present disclosure will employ, unless otherwise indicated, conventional techniques of botany, microbiology, tissue culture, molecular biology, chemistry, biochemistry and recombinant DNA technology, which are within the skill of the art.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present disclosure, the following terms will be employed and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., Diagnostic Molecular Microbiology: Principles and Applications, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32)) can be modified to yield a functionally identical molecule. Accordingly, each silent variation of a nucleic acid, which encodes a polypeptide of the present disclosure, is implicit in each described polypeptide sequence and incorporated herein by reference.

As to amino acid sequences, one of skill will recognize that individual substitution, deletion or addition to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity or ligand/receptor binding is generally at least 30%, 40%, 50%, 60%, 70%, 80% or 90%, preferably 60-90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V) and

6) Phenylalanine (F), Tyrosine (Y), Tryptophan (W).

See also, Creighton, Proteins, W.H. Freeman and Co. (1984).

As used herein, “consisting essentially of” means the inclusion of additional sequences to an object polynucleotide or polypeptide where the additional sequences do not materially affect the basic function of the claimed polynucleotide or polypeptide sequences.

The term “construct” is used to refer generally to an artificial combination of polynucleotide sequences, i.e. a combination which does not occur in nature, normally comprising one or more regulatory elements and one or more coding sequences. The term may include reference to expression cassettes and/or vector sequences, as is appropriate for the context.

A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed. A control plant may also be a plant transformed with an alternative construct.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, the term “endogenous”, when used in reference to a gene, means a gene that is normally present in the genome of cells of a specified organism and is present in its normal state in the cells (i.e., present in the genome in the state in which it normally is present in nature).

The term “exogenous” is used herein to refer to any material that is introduced into a cell. The term “exogenous nucleic acid molecule” or “transgene” refers to any nucleic acid molecule that either is not normally present in a cell genome or is introduced into a cell. Such exogenous nucleic acid molecules generally are recombinant nucleic acid molecules, which are generated using recombinant DNA methods as disclosed herein or otherwise known in the art. In various embodiments, a transgenic non-human organism as disclosed herein, can contain, for example, a first transgene and a second transgene. Such first and second transgenes can be introduced into a cell, for example, a progenitor cell of a transgenic organism, either as individual nucleic acid molecules or as a single unit (e.g., contained in different vectors or contained in a single vector, respectively). In either case, confirmation may be made that a cell from which the transgenic organism is to be derived contains both of the transgenes using routine and well-known methods such as expression of marker genes or nucleic acid hybridization or PCR analysis. Alternatively, or additionally, confirmation of the presence of transgenes may occur later, for example, after regeneration of a plant from a putatively transformed cell.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell which comprises a heterologous nucleic acid sequence of the disclosure, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected m RNA).

The terms “isolated” refers to material, such as a nucleic acid or a protein, which is substantially or essentially free from components which normally accompany or interact with it as found in its naturally occurring environment. The terms “non-naturally occurring”; “mutated”, “recombinant”; “recombinantly expressed”; “heterologous” or “heterologously expressed” are representative biological materials that are not present in its naturally occurring environment.

By “line” with reference to plants is meant a collection of genetically identical plants.

The term “NUE nucleic acid” means a nucleic acid comprising a polynucleotide (“NUE polynucleotide”) encoding a full length or partial length polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3 and Current Protocols in Molecular Biology, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter, and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, a cell present in or isolated from plant tissues including seeds, suspension cultures, embryos, meristematic regions, callus tissue, leaves, roots, shoots, gametophytes, sporophytes, pollen and microspores. The class of plants which can be used in the methods of the disclosure is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” may include reference to bushels per acre of a grain crop at harvest, as adjusted for grain moisture (15% typically for maize, for example) and/or the volume of biomass generated (for forage crops such as alfalfa and plant root size for multiple crops). Grain moisture is measured in the grain at harvest. The adjusted test weight of grain is determined to be the weight in pounds per bushel, adjusted for grain moisture level at harvest. Biomass is measured as the weight of harvestable plant material generated.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term may include reference to the specified sequence as well as the complementary sequence thereof.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such as Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters are members of the class of “non-constitutive” promoters. A “constitutive” promoter is a promoter which is active in essentially all tissues of a plant, under most environmental conditions and states of development or cell differentiation.

The term “polypeptide” refers to one or more amino acid sequences. The term is also inclusive of fragments, variants, homologs, alleles or precursors (e.g., preproproteins or proproteins) thereof. A “NUE protein” comprises a polypeptide. Unless otherwise stated, the term “NUE nucleic acid” means a nucleic acid comprising a polynucleotide (“NUE polynucleotide”) encoding a polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C. and a wash in 0.1×SSC, 0.1% SDS at 65° C.

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, Current Protocols in Molecular Biology, Chapter 19, Ausubel et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, optionally at least 50%, at least 60%, at least 70%, at least 80%, at least 90%, or at least 95% sequence identity, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, such as at least 55%, at least 60%, at least 70%, at least 80%, at least 90%, at least 95%, up to 100% identity.

The terms “substantial identity” in the context of a peptide indicates that a peptide comprises a sequence with between 55-100% sequence identity to a reference sequence, such as at least 55%, at least 60%, at least 70%, at least 80%, at least 90%, at least 95%, up to 100% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides which are “substantially similar” share sequences as noted above, except that residue positions, which are not identical, may differ by conservative amino acid changes.

### Construction of Nucleic Acids

The isolated nucleic acids of the present disclosure can be made using (a) standard recombinant methods, (b) synthetic techniques or combinations thereof. In some embodiments, the polynucleotides of the present disclosure will be cloned, amplified or otherwise constructed from a fungus or bacteria.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

Further, the polypeptide-encoding segments of the polynucleotides of the present disclosure can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present disclosure can be analyzed statistically using commercially available software packages such as “Codon Preference” available from the University of Wisconsin Genetics Computer Group. See, Devereaux, et al., (1984) Nucleic Acids Res. 12:387-395) or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides (3 nucleotides per amino acid) that can be used to determine a codon usage frequency can be any integer from 3 to the number of polynucleotides of the present disclosure as provided herein. Optionally, the polynucleotides will be full-length sequences. An exemplary number of sequences for statistical analysis can be at least 1, 5, 10, 20, 50 or 100.

### Sequence Shuffling

The present disclosure provides methods for sequence shuffling using polynucleotides of the present disclosure, and compositions resulting therefrom. Sequence shuffling is described in PCT Publication Number 1996/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present disclosure further provides recombinant expression cassettes comprising a nucleic acid of the present disclosure. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

### Promoters, Terminators, Introns

A plant promoter fragment can be employed which will direct expression of a polynucleotide of the present disclosure in essentially all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 1996/30530 and other transcription initiation regions from various plant genes known to those of skill. For the present disclosure ubiquitin is the preferred promoter for expression in monocot plants.

Alternatively, the plant promoter can direct expression of a polynucleotide of the present disclosure in a specific tissue or may be otherwise under more precise environmental or developmental control. Such promoters may be “inducible” promoters. Environmental conditions that may affect transcription by inducible promoters include pathogen attack, anaerobic conditions or the presence of light. Examples of inducible promoters are the Adh1 promoter, which is inducible by hypoxia or cold stress, the Hsp70 promoter, which is inducible by heat stress and the PPDK promoter, which is inducible by light. Diurnal promoters that are active at different times during the circadian rhythm are also known (US Patent Application Publication Number 2011/0167517, incorporated herein by reference).

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, The Maize Handbook, Chapter 116, Freeling and Walbot, eds., Springer, New York (1994).

### Signal Peptide Sequences

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119) or signal peptides which target proteins to the plastids such as that of rapeseed enoyl-Acp reductase (Verwaert, et al., (1994) Plant Mol. Biol. 26:189-202) are useful in the disclosure.

### Markers

The vector comprising the sequences from a polynucleotide of the present disclosure will typically comprise a marker gene, which confers a selectable phenotype on plant cells. The selectable marker gene may encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance. Also useful are genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron.

Constructs described herein may comprise a polynucleotide of interest encoding a reporter or marker product. Examples of suitable reporter polynucleotides known in the art can be found in, for example, Jefferson, et al., (1991) in Plant Molecular Biology Manual, ed. Gelvin, et al., (Kluwer Academic Publishers), pp. 1-33; DeWet, et al. (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330. In certain embodiments, the polynucleotide of interest encodes a selectable reporter. These can include polynucleotides that confer antibiotic resistance or resistance to herbicides. Examples of suitable selectable marker polynucleotides include, but are not limited to, genes encoding resistance to chloramphenicol, methotrexate, hygromycin, streptomycin, spectinomycin, bleomycin, sulfonamide, bromoxynil, glyphosate and phosphinothricin.

In some embodiments, the expression cassettes disclosed herein comprise a polynucleotide of interest encoding scorable or screenable markers, where presence of the polynucleotide produces a measurable product. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase and alkaline phosphatase. Other screenable markers include the anthocyanin/flavonoid polynucleotides including, for example, a R-locus polynucleotide, which encodes a product that regulates the production of anthocyanin pigments (red color) in plant tissues, the genes which control biosynthesis of flavonoid pigments, such as the maize C1 and C2, the B gene, the p1 gene and the bronze locus genes, among others. Further examples of suitable markers encoded by polynucleotides of interest include the cyan fluorescent protein (CYP) gene, the yellow fluorescent protein gene, a lux gene, which encodes a luciferase, the presence of which may be detected using, for example, X-ray film, scintillation counting, fluorescent spectrophotometry, low-light video cameras, photon counting cameras or multiwell luminometry, a green fluorescent protein (GFP) and DsRed2 (Clontechniques, 2001) where plant cells transformed with the marker gene are red in color, and thus visually selectable. Additional examples include a p-lactamase gene encoding an enzyme for which various chromogenic substrates are known (e.g., PADAC, a chromogenic cephalosporin), a xylE gene encoding a catechol dioxygenase that can convert chromogenic catechols, an α-amylase gene and a tyrosinase gene encoding an enzyme capable of oxidizing tyrosine to DOPA and dopaquinone, which in turn condenses to form the easily detectable compound melanin.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su, et al., (2004) Biotechnol Bioeng 85:610-9 and Fetter, et al., (2004) Plant Cell 16:215-28), cyan florescent protein (CYP) (Bolte, et al., (2004) J. Cell Science 117:943-54 and Kato, et al., (2002) Plant Physiol 129:913-42) and yellow florescent protein (PhiYFP™ from Evrogen, see, Bolte, et al., (2004) J. Cell Science 117:943-54). For additional selectable markers, see generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Aci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Bairn, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the compositions and methods disclosed herein.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present disclosure, one may express a protein of the present disclosure in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present disclosure. No attempt to describe in detail the various methods known for the expression of proteins in prokaryotes or eukaryotes will be made.

In brief summary, the expression of isolated nucleic acids encoding a protein of the present disclosure will typically be achieved by operably linking, for example, the DNA or cDNA to a promoter, followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA of the present disclosure. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

One of skill would recognize that modifications could be made to a protein of the present disclosure without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present disclosure are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present disclosure.

### Expression in Eukaryotes

A variety of eukaryotic expression systems such as yeast, insect cell lines, plant and mammalian cells, are known to those of skill in the art. As explained briefly below, the present disclosure can be expressed in these eukaryotic systems. In some embodiments, transformed/transfected plant cells, as discussed infra, are employed as expression systems for production of the proteins of the instant disclosure.

Synthesis of heterologous proteins in yeast is well known. Sherman, et al., (1982) Methods in Yeast Genetics, Cold Spring Harbor Laboratory is a well-recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present disclosure, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present disclosure can also be ligated to various expression vectors for use in transfecting cell cultures of, for instance, mammalian, insect or plant origin. Mammalian cell systems often will be in the form of monolayers of cells although mammalian cell suspensions may also be used. A number of suitable host cell lines capable of expressing intact proteins have been developed in the art, and include the HEK293, BHK21 and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins of the present disclosure are available, for instance, from the American Type Culture Collection Catalogue of Cell Lines and Hybridomas (7^{th} ed., 1992).

Appropriate vectors for expressing proteins of the present disclosure in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al., (1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA Cloning: A Practical Approach, vol. II, Glover, ed., IRL Press, Arlington, Va., pp. 213-38 (1985)).

In addition, the gene of interest placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing heterologous genes into plants are known and can be used to insert a polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant and include chemical transfection methods such as calcium phosphate, microorganism-mediated gene transfer such as Agrobacterium (Horsch, et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. Gamborg and Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 1991/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas, (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209. Longman, N Y (pollen); Kaeppler, et al., (1990) Plant Cell Reports 9:415-418 and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive or tissue-preferred expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once constructed, these plasmids can be placed into A. rhizogenes or A. tumefaciens and these vectors used to transform cells of plant species which are ordinarily susceptible to Fusarium or Alternaria infection. Several other transgenic plants are also contemplated by the present disclosure including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can also be transformed. EP Patent Application Number 604 662 A1 discloses a method for transforming monocots using Agrobacterium. EP Patent Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue in the form of an explant, such as cotyledonary tissue or leaf disks, can be inoculated with these vectors, and cultured under conditions which promote plant regeneration. Examples of such methods for regenerating plant tissue are disclosed in Shahin, (1985) Theor. Appl. Genet. 69:235-40; U.S. Pat. No. 4,658,082; Simpson, et al., supra and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Reducing the Activity and/or Level of a Polypeptide

Methods are provided to reduce or eliminate the activity of a polypeptide of the disclosure by transforming a plant cell with an expression cassette that expresses a polynucleotide that inhibits the expression of the polypeptide. The polynucleotide may inhibit the expression of the polypeptide directly, by preventing transcription or translation of the messenger RNA, or indirectly, by encoding a polypeptide that inhibits the transcription or translation of a gene encoding polypeptide. Methods for inhibiting or eliminating the expression of a gene in a plant are well known in the art and any such method may be used in the present disclosure to inhibit the expression of polypeptide.

In accordance with the present disclosure, the expression of a polypeptide may be inhibited so that the protein level of the polypeptide is, for example, less than 70% of the protein level of the same polypeptide in a plant that has not been genetically modified or mutagenized to inhibit the expression of that polypeptide. In particular embodiments of the disclosure, the protein level of the polypeptide in a modified plant according to the disclosure is less than 60%, less than 50%, less than 40%, less than 30%, less than 20%, less than 10%, less than 5% or less than 2% of the protein level of the same polypeptide in a plant that is not a mutant or that has not been genetically modified to inhibit the expression of that polypeptide. The expression level of the polypeptide may be measured directly, for example, by assaying for the level of polypeptide expressed in the plant cell or plant, or indirectly, for example, by measuring the nitrogen uptake activity of the polypeptide in the plant cell or plant or by measuring the phenotypic changes in the plant. Methods for performing such assays are described elsewhere herein.

In other embodiments of the disclosure, the activity of the polypeptide is reduced or eliminated by transforming a plant cell with an expression cassette comprising a polynucleotide encoding a polypeptide that inhibits the activity of a polypeptide. The activity of a polypeptide is inhibited according to the present disclosure if the activity of the polypeptide is, for example, less than 70% of the activity of the same polypeptide in a plant that has not been modified to inhibit the activity of that polypeptide. In particular embodiments of the disclosure, the activity of the polypeptide in a modified plant according to the disclosure is less than 60%, less than 50%, less than 40%, less than 30%, less than 20%, less than 10% or less than 5% of the activity of the same polypeptide in a plant that that has not been modified to inhibit the expression of that polypeptide. The activity of a polypeptide is “eliminated” according to the disclosure when it is not detectable by the assay methods described elsewhere herein. Methods of determining the alteration of activity of a polypeptide are described elsewhere herein.

In other embodiments, the activity of a polypeptide may be reduced or eliminated by disrupting the gene encoding the polypeptide. The disclosure encompasses mutagenized plants that carry mutations in genes, where the mutations reduce expression of the gene or inhibit the activity of the encoded polypeptide.

Thus, many methods may be used to reduce or eliminate the activity of a polypeptide. In addition, more than one method may be used to reduce the activity of a single polypeptide.

### 1. Polynucleotide-Based Methods:

In some embodiments of the present disclosure, a plant is transformed with an expression cassette that is capable of expressing a polynucleotide that inhibits the expression of a polypeptide of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one polypeptide is an expression cassette capable of producing an RNA molecule that inhibits the transcription and/or translation of at least one polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a polypeptide are given below.

i. Sense Suppression/Cosuppression

In some embodiments of the disclosure, inhibition of the expression of a polypeptide may be obtained by sense suppression or cosuppression. For cosuppression, an expression cassette is designed to express an RNA molecule corresponding to all or part of a messenger RNA encoding a polypeptide in the “sense” orientation. Over expression of the RNA molecule can result in reduced expression of the native gene. Accordingly, multiple plant lines transformed with the cosuppression expression cassette are screened to identify those that show the desired degree of inhibition of polypeptide expression.

The polynucleotide used for cosuppression may correspond to all or part of the sequence encoding the polypeptide, all or part of the 5′ and/or 3′ untranslated region of a polypeptide transcript or all or part of both the coding sequence and the untranslated regions of a transcript encoding a polypeptide. In some embodiments where the polynucleotide comprises all or part of the coding region for the polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323, herein incorporated by reference.

ii. Antisense Suppression

In some embodiments of the disclosure, inhibition of the expression of the polypeptide may be obtained by antisense suppression. For antisense suppression, the expression cassette is designed to express an RNA molecule complementary to all or part of a messenger RNA encoding the polypeptide. Over expression of the antisense RNA molecule can result in reduced expression of the target gene. Accordingly, multiple plant lines transformed with the antisense suppression expression cassette are screened to identify those that show the desired degree of inhibition of polypeptide expression.

The polynucleotide for use in antisense suppression may correspond to all or part of the complement of the sequence encoding the polypeptide, all or part of the complement of the 5′ and/or 3′ untranslated region of the target transcript or all or part of the complement of both the coding sequence and the untranslated regions of a transcript encoding the polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the disclosure, inhibition of the expression of a polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the desired degree of inhibition of polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

iv. Hairpin RNA Interference and Intron-Containing Hairpin RNA Interference

In some embodiments of the disclosure, inhibition of the expression of a polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoded by the gene whose expression is to be inhibited, and an antisense sequence that is fully or partially complementary to the sense sequence. Alternatively, the base-paired stem region may correspond to a portion of a promoter sequence controlling expression of the gene whose expression is to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., Curr. Biol. (2001) 11:436-440), herein incorporated by reference.

v. Amplicon-Mediated Interference

Amplicon expression cassettes comprise a plant-virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the disclosure is catalytic RNA or has ribozyme activity specific for the messenger RNA of the polypeptide. Thus, the polynucleotide causes the degradation of the endogenous messenger RNA, resulting in reduced expression of the polypeptide. This method is described, for example, in U.S. Pat. No. 4,987,071, herein incorporated by reference.

vii. Small Interfering RNA or Micro RNA

In some embodiments of the disclosure, inhibition of the expression of a polypeptide may be obtained by RNA interference by expression of a polynucleotide encoding a micro RNA (miRNA). miRNAs are regulatory agents consisting of about 22 ribonucleotides. miRNA are highly efficient at inhibiting the expression of endogenous genes. See, for example Javier, et al., (2003) Nature 425:257-263, herein incorporated by reference.

For miRNA interference, the expression cassette is designed to express an RNA molecule that is modeled on an endogenous miRNA gene. For example, the miRNA gene encodes an RNA that forms a hairpin structure containing a 22-nucleotide sequence that is complementary to an endogenous gene target sequence. For suppression of NUE expression, the 22-nucleotide sequence is selected from a NUE transcript sequence and contains 22 nucleotides of said NUE sequence in sense orientation and 21 nucleotides of a corresponding antisense sequence that is complementary to the sense sequence. A fertility gene, whether endogenous or exogenous, may be a miRNA target. miRNA molecules are highly efficient at inhibiting the expression of endogenous genes, and the RNA interference they induce is inherited by subsequent generations of plants.

### 2. Polypeptide-Based Inhibition of Gene Expression

In one embodiment, the polynucleotide encodes a zinc finger protein that binds to a gene encoding a polypeptide, resulting in reduced expression of the gene. In particular embodiments, the zinc finger protein binds to a regulatory region of a gene. In other embodiments, the zinc finger protein binds to a messenger RNA encoding a polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US Patent Application Publication Number 2003/0037355, each of which is herein incorporated by reference.

### 3. Polypeptide-Based Inhibition of Protein Activity

In some embodiments of the disclosure, the polynucleotide encodes an antibody that binds to at least one polypeptide and reduces the activity of the polypeptide. In another embodiment, the binding of the antibody results in increased turnover of the antibody-polypeptide complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

### 4. Gene Disruption

In some embodiments of the present disclosure, the activity of a polypeptide is reduced or eliminated by disrupting the gene encoding the polypeptide. The gene encoding the polypeptide may be disrupted by any method known in the art. For example, in one embodiment, the gene is disrupted by transposon tagging. In another embodiment, the gene is disrupted by mutagenizing plants using random or targeted mutagenesis and selecting for plants that have reduced nitrogen utilization activity.

i. Transposon Tagging

In one embodiment of the disclosure, transposon tagging is used to reduce or eliminate the activity of one or more polypeptide. Transposon tagging comprises inserting a transposon within an endogenous gene to reduce or eliminate expression of the polypeptide.

In this embodiment, the expression of one or more polypeptides is reduced or eliminated by inserting a transposon within a regulatory region or coding region of the gene encoding the polypeptide. A transposon that is within an exon, intron, 5′ or 3′ untranslated sequence, a promoter or any other regulatory sequence of a gene may be used to reduce or eliminate the expression and/or activity of the encoded polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764, each of which is herein incorporated by reference.

ii. Mutant Plants with Reduced Activity

Additional methods for decreasing or eliminating the expression of endogenous genes in plants are known in the art and can be similarly applied to the instant disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See, McCallum, et al., (2000) Nat. Biotechnol. 18:455-457, herein incorporated by reference.

Mutations that impact gene expression or that interfere with the function of the encoded protein are well known in the art. Insertional mutations in gene exons usually result in null-mutants. Mutations in conserved residues are particularly effective in inhibiting the activity of the encoded protein. Conserved residues of plant polypeptides suitable for mutagenesis with the goal to eliminate activity have been described. Such mutants can be isolated according to well-known procedures and mutations in different loci can be stacked by genetic crossing. See, for example, Gruis, et al., (2002) Plant Cell 14:2863-2882.

In another embodiment of this disclosure, dominant mutants can be used to trigger RNA silencing due to gene inversion and recombination of a duplicated gene locus. See, for example, Kusaba, et al., (2003) Plant Cell 15:1455-1467.

The disclosure encompasses additional methods for reducing or eliminating the activity of one or more polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 1998/49350, WO 1999/07865, WO 1999/25821 and Beetham, et al., (1999) Proc. Natl. Acad. Sci. USA 96:8774-8778, each of which is herein incorporated by reference.

iii. Modulating Nitrogen Utilization Activity

In specific methods, the level and/or activity of a NUE regulator in a plant is decreased by increasing the level or activity of the polypeptide in the plant. The increased expression of a negative regulatory molecule may decrease the level of expression of downstream one or more genes responsible for an improved NUE phenotype.

Methods for increasing the level and/or activity of polypeptides in a plant are discussed elsewhere herein. Briefly, such methods comprise providing a polypeptide of the disclosure to a plant and thereby increasing the level and/or activity of the polypeptide. In other embodiments, a NUE nucleotide sequence encoding a polypeptide can be provided by introducing into the plant a polynucleotide comprising a NUE nucleotide sequence of the disclosure, expressing the NUE sequence, increasing the activity of the polypeptide and thereby decreasing the number of tissue cells in the plant or plant part. In other embodiments, the NUE nucleotide construct introduced into the plant is stably incorporated into the genome of the plant.

In other methods, the growth of a plant tissue is increased by decreasing the level and/or activity of the polypeptide in the plant. Such methods are disclosed in detail elsewhere herein. In one such method, a NUE nucleotide sequence is introduced into the plant and expression of said NUE nucleotide sequence decreases the activity of the polypeptide and thereby increasing the tissue growth in the plant or plant part. In other embodiments, the NUE nucleotide construct introduced into the plant is stably incorporated into the genome of the plant.

As discussed above, one of skill will recognize the appropriate promoter to use to modulate the level/activity of a NUE in the plant. Exemplary promoters for this embodiment have been disclosed elsewhere herein.

In other embodiments, such plants have stably incorporated into their genome a nucleic acid molecule comprising a NUE nucleotide sequence of the disclosure operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the polypeptide in the plant. In one method, a sequence of the disclosure is provided to the plant. In another method, the nucleotide sequence is provided by introducing into the plant a polynucleotide comprising a nucleotide sequence of the disclosure, expressing the sequence and thereby modifying root development. In still other methods, the nucleotide construct introduced into the plant is stably incorporated into the genome of the plant.

In other methods, root development is modulated by altering the level or activity of the polypeptide in the plant. A change in activity can result in at least one or more of the following alterations to root development, including, but not limited to, alterations in root biomass and length.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by decreasing the activity and/or level of the polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse environmental conditions. This trait relates to the size, depth and morphology of the root system. In addition, stimulating root growth and increasing root mass by altering the level and/or activity of the polypeptide finds use in promoting in vitro propagation of explants.

Furthermore, higher root biomass production has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present disclosure further provides plants having modulated root development when compared to the root development of a control plant. In some embodiments, the plant of the disclosure has an increased level/activity of a polypeptide of the disclosure and has enhanced root growth and/or root biomass. In other embodiments, such plants have stably incorporated into their genome a nucleic acid molecule comprising a nucleotide sequence of the disclosure operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a polypeptide of the disclosure. In one embodiment, a sequence of the disclosure is provided. In other embodiments, the nucleotide sequence can be provided by introducing into the plant a polynucleotide comprising a nucleotide sequence of the disclosure, expressing the sequence and thereby modifying shoot and/or leaf development. In other embodiments, the nucleotide construct introduced into the plant is stably incorporated into the genome of the plant.

In specific embodiments, shoot or leaf development is modulated by altering the level and/or activity of the polypeptide in the plant. A change in activity can result in at least one or more of the following alterations in shoot and/or leaf development, including, but not limited to, changes in leaf number, altered leaf surface, altered vasculature, internodes and plant growth and alterations in leaf senescence when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters and leaf-preferred promoters. Exemplary promoters have been disclosed elsewhere herein.

Increasing activity and/or level of a polypeptide of the disclosure in a plant may result in altered internodes and growth. Thus, the methods of the disclosure find use in producing modified plants. In addition, as discussed above, activity in the plant modulates both root and shoot growth. Thus, the present disclosure further provides methods for altering the root/shoot ratio. Shoot or leaf development can further be modulated by altering the level and/or activity of the polypeptide in the plant.

Accordingly, the present disclosure further provides plants having modulated shoot and/or leaf development when compared to a control plant. In some embodiments, the plant of the disclosure has an increased level/activity of a polypeptide of the disclosure. In other embodiments, a plant of the disclosure has a decreased level/activity of a polypeptide of the disclosure.

vi. Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the polypeptide has not been modulated. “Modulating floral development” further includes any alteration in the timing of the development of a plant's reproductive tissue (e.g., a delayed or an accelerated timing of floral development) when compared to a control plant in which the activity or level of the polypeptide has not been modulated. Changes in timing of reproductive development may result in altered synchronization of development of male and female reproductive tissues. Macroscopic alterations may include changes in size, shape, number or location of reproductive organs, the developmental time period that these structures form or the ability to maintain or proceed through the flowering process in times of environmental stress. Microscopic alterations may include changes to the types or shapes of cells that make up the reproductive organs.

The method for modulating floral development in a plant comprises modulating activity in a plant. In one method, a sequence of the disclosure is provided. A nucleotide sequence can be provided by introducing into the plant a polynucleotide comprising a nucleotide sequence of the disclosure, expressing the sequence and thereby modifying floral development. In other embodiments, the nucleotide construct introduced into the plant is stably incorporated into the genome of the plant.

In specific methods, floral development is modulated by increasing the level or activity of the polypeptide in the plant. A change in activity can result in at least one or more of the following alterations in floral development, including, but not limited to, altered flowering, changed number of flowers, modified male sterility and altered seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

In other methods, floral development is modulated by altering the level and/or activity of a sequence of the disclosure. Such methods can comprise introducing a nucleotide sequence into the plant and changing the activity of the polypeptide. In other methods, the nucleotide construct introduced into the plant is stably incorporated into the genome of the plant. Altering expression of the sequence of the disclosure can modulate floral development during periods of stress. Such methods are described elsewhere herein. Accordingly, the present disclosure further provides plants having modulated floral development when compared to the floral development of a control plant. Compositions include plants having an altered level/activity of the polypeptide of the disclosure and having an altered floral development. Compositions also include plants having a modified level/activity of the polypeptide of the disclosure wherein the plant maintains or proceeds through the flowering process in times of stress.

Methods are also provided for the use of the sequences of the disclosure to increase seed size and/or weight. The method comprises increasing the activity of the sequences in a plant or plant part, such as the seed. An increase in seed size and/or weight comprises an increased size or weight of the seed and/or an increase in the size or weight of one or more seed part including, for example, the embryo, endosperm, seed coat, aleurone or cotyledon.

As discussed above, one of skill will recognize the appropriate promoter to use to increase seed size and/or seed weight. Exemplary promoters of this embodiment include constitutive promoters, inducible promoters, seed-preferred promoters, embryo-preferred promoters and endosperm-preferred promoters.

A method for altering seed size and/or seed weight in a plant may increasing activity in the plant. In one embodiment, the nucleotide sequence can be provided by introducing into the plant a polynucleotide comprising a nucleotide sequence of the disclosure, expressing the sequence and thereby impacting seed weight and/or size. In certain embodiments, the nucleotide construct introduced into the plant is stably incorporated into the genome of the plant.

It is further recognized that increasing seed size and/or weight can also be accompanied by an increase in the speed of growth of seedlings or an increase in early vigor. As used herein, the term “early vigor” refers to the ability of a plant to grow rapidly during early development, and relates to the successful establishment, after germination, of a well-developed root system and a well-developed photosynthetic apparatus. In addition, an increase in seed size and/or weight can also result in an increase in plant yield when compared to a control.

Accordingly, the present disclosure further provides plants having an increased seed weight and/or seed size when compared to a control plant. In other embodiments, plants having an increased vigor and plant yield are also provided. In some embodiments, the plant of the disclosure has a modified level/activity of the polypeptide of the disclosure and has an increased seed weight and/or seed size. In other embodiments, such plants have stably incorporated into their genome a nucleic acid molecule comprising a nucleotide sequence of the disclosure operably linked to a promoter that drives expression in the plant cell.

vii. Method of Use for Polynucleotide, Expression Cassettes, and Additional Polynucleotides

The nucleotides, expression cassettes and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increases, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading and the like.

In certain embodiments the nucleic acid sequences of the present disclosure can be used in combination (“stacked”) with other polynucleotide sequences of interest in order to create plants with a desired phenotype. The combinations generated can include multiple copies of any one or more of the polynucleotides of interest. The stacked polynucleotides or constructs may target genes of the same family, or target genes within the same biosynthetic pathway. Such stacking may amplify a desired impact, response, or phenotype.

The promoter which is operably linked to a polynucleotide sequence of interest can be any promoter that is active in plant cells. In some embodiments it is particularly advantageous to use a promoter that is active (or can be activated) in reproductive tissues of a plant (e.g., stamens or ovaries). As such, the promoter can be, for example, a constitutively active promoter, an inducible promoter, a tissue-specific promoter or a developmental stage specific promoter. Also, the promoter of a exogenous nucleic acid molecule can be the same as or different from the promoter of a second exogenous nucleic acid molecule.

The polynucleotides of the present disclosure may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present disclosure can also be stacked with traits desirable for insect, disease or herbicide resistance (e.g., Bacillus thuringiensis toxic proteins (U.S. Pat. Nos. 5,366,892; 5,747,450; 5,737,514; 5,723,756; U.S. Pat. No. 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present disclosure with polynucleotides affecting agronomic traits such as male sterility (e.g., see, U.S. Pat. No. 5,583,210), stalk strength, flowering time or transformation technology traits such as cell cycle regulation or gene targeting (e.g., WO 1999/61619; WO 2000/17364; WO 1999/25821), the disclosures of which are herein incorporated by reference.

Transgenic plants comprising or derived from plant cells or native plants of this disclosure can be further enhanced with stacked traits, e.g., a crop plant having an enhanced trait resulting from expression of DNA disclosed herein in combination with herbicide tolerance and/or pest resistance traits. For example, plants with an altered trait of interest can be stacked with other traits of agronomic interest, such as a trait providing herbicide resistance and/or insect resistance, such as using a gene from Bacillus thuringensis to provide resistance against one or more of lepidopteran, coleopteran, homopteran, hemiopteran and other insects. Known genes that confer tolerance to herbicides such as e.g., auxin, HPPD, glyphosate, dicamba, glufosinate, sulfonylurea, bromoxynil and norflurazon herbicides can be stacked either as a molecular stack or a breeding stack with plants expressing the traits disclosed herein. Polynucleotide molecules encoding proteins involved in herbicide tolerance include, but are not limited to, a polynucleotide molecule encoding 5-enolpyruvylshikimate-3-phosphate synthase (EPSPS) disclosed in U.S. Pat. Nos. 39,247; 6,566,587 and for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Pat. Nos. 7,622,641; 7,462,481; 7,531,339; 7,527,955; 7,709,709; 7,714,188 and 7,666,643, also for providing glyphosate tolerance; dicamba monooxygenase disclosed in U.S. Pat. No. 7,022,896 and WO 2007/146706 A2 for providing dicamba tolerance; a polynucleotide molecule encoding AAD12 disclosed in US Patent Application Publication Number 2005/731044 or WO 2007/053482 A2 or encoding AAD1 disclosed in US Patent Application Publication Number 2011/0124503 A1 or U.S. Pat. No. 7,838,733 for providing tolerance to auxin herbicides (2,4-D); a polynucleotide molecule encoding hydroxyphenylpyruvate dioxygenase (HPPD) for providing tolerance to HPPD inhibitors (e.g., hydroxyphenylpyruvate dioxygenase) disclosed in e.g., U.S. Pat. No. 7,935,869; US Patent Application Publication Numbers 2009/0055976 A1 and 2011/0023180 A1; each publication is herein incorporated by reference in its entirety.

Other examples of herbicide-tolerance traits that could be combined with the traits disclosed herein include those conferred by polynucleotides encoding an exogenous phosphinothricin acetyltransferase, as described in U.S. Pat. Nos. 5,969,213; 5,489,520; 5,550,318; 5,874,265; 5,919,675; 5,561,236; 5,648,477; 5,646,024; 6,177,616 and 5,879,903. Plants containing an exogenous phosphinothricin acetyltransferase can exhibit improved tolerance to glufosinate herbicides, which inhibit the enzyme glutamine synthase. Other examples of herbicide-tolerance traits include those conferred by polynucleotides conferring altered protoporphyrinogen oxidase (protox) activity, as described in U.S. Pat. Nos. 6,288,306 B1; 6,282,837 B1 and 5,767,373 and international publication WO 2001/12825. Plants containing such polynucleotides can exhibit improved tolerance to any of a variety of herbicides which target the protox enzyme (also referred to as “protox inhibitors”)

In one embodiment, sequences of interest improve plant growth and/or crop yields. For example, sequences of interest include agronomically important genes that result in improved primary or lateral root systems. Such genes include, but are not limited to, nutrient/water transporters and growth inducers. Examples of such genes include, but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that negatively affect root development.

Additional, agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley described in Williamson, et al., (1987) Eur. J. Biochem. 165:99-106, the disclosures of which are herein incorporated by reference. Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996, and WO 1998/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen, et al., (1986) J. Biol. Chem. 261:6279; Kirihara, et al., (1988) Gene 71:359, both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109) and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089) and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical emasculation. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase, for example, starch for ethanol production or provide expression of proteins. Another important commercial use of transformed plants is the production of polymers and bioplastics such as described in U.S. Pat. No. 5,602,321. Genes such as 13-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

Genome Editing and Induced Mutagenesis

In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein is generated using “custom” meganucleases produced to modify plant genomes (see, e.g., WO 2009/114321; Gao, et al., (2010) Plant Journal 1:176-187). Other site-directed engineering is through the use of zinc finger domain recognition coupled with the restriction properties of restriction enzyme. See, e.g., Urnov, et al., (2010) Nat Rev Genet. 11(9):636-46; Shukla, et al., (2009) Nature 459(7245):437-41.

“TILLING” or “Targeting Induced Local Lesions IN Genomics” refers to a mutagenesis technology useful to generate and/or identify and to eventually isolate mutagenised variants of a particular nucleic acid with modulated expression and/or activity (McCallum, et al., (2000), Plant Physiology 123:439-442; McCallum, et al., (2000) Nature Biotechnology 18:455-457 and Colbert, et al., (2001) Plant Physiology 126:480-484).

TILLING combines high density point mutations with rapid sensitive detection of the mutations. Typically, ethylmethanesulfonate (EMS) is used to mutagenize plant seed. EMS alkylates guanine, which typically leads to mispairing. For example, seeds are soaked in an about 10-20 mM solution of EMS for about 10 to 20 hours; the seeds are washed and then sown. The plants of this generation are known as M1. M1 plants are then self-fertilized. Mutations that are present in cells that form the reproductive tissues are inherited by the next generation (M2). Typically, M2 plants are screened for mutation in the desired gene and/or for specific phenotypes.

TILLING also allows selection of plants carrying mutant variants. These mutant variants may exhibit modified expression, either in strength or in location or in timing (if the mutations affect the promoter, for example). These mutant variants may exhibit higher or lower activity than that exhibited by the gene in its natural form. TILLING combines high-density mutagenesis with high-throughput screening methods. The steps typically followed in TILLING are: (a) EMS mutagenesis (Redei and Koncz, (1992) In Methods in Arabidopsis Research, Koncz, et al., eds. Singapore, World Scientific Publishing Co, pp. 16-82; Feldmann, et al., (1994) In Arabidopsis. Meyerowitz and Somerville, eds, Cold Spring Harbor Laboratory Press, Cold Spring Harbor, N.Y., pp 137-172; Lightner and Caspar, (1998) In Methods on Molecular Biology 82:91-104; Martinez-Zapater and Salinas, eds, Humana Press, Totowa, N.J.); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (U.S. Pat. No. 8,071,840).

Other mutagenic methods can also be employed to introduce mutations in a disclosed gene. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

Embodiments of the disclosure reflect the determination that the genotype of an organism can be modified to contain dominant suppressor alleles or transgene constructs that suppress (i.e., reduce, but not ablate) the activity of a gene, wherein the phenotype of the organism is not substantially affected.

Hybrid seed production requires elimination or inactivation of pollen produced by the female parent. Incomplete removal or inactivation of the pollen provides the potential for selfing, raising the risk that inadvertently self-pollinated seed will unintentionally be harvested and packaged with hybrid seed. Once the seed is planted, the selfed plants can be identified and selected; the selfed plants are genetically equivalent to the female inbred line used to produce the hybrid. Typically, the selfed plants are identified and selected based on their decreased vigor relative to the hybrid plants. For example, female selfed plants of maize are identified by their less vigorous appearance for vegetative and/or reproductive characteristics, including shorter plant height, small ear size, ear and kernel shape, cob color or other characteristics. Selfed lines also can be identified using molecular marker analyses (see, e.g., Smith and Wych, (1995) Seed Sci. Technol. 14:1-8). Using such methods, the homozygosity of the self-pollinated line can be verified by analyzing allelic composition at various loci in the genome.

Because hybrid plants are important and valuable field crops, plant breeders are continually working to develop high-yielding hybrids that are agronomically sound based on stable inbred lines. The availability of such hybrids allows a maximum amount of crop to be produced with the inputs used, while minimizing susceptibility to pests and environmental stresses. To accomplish this goal, the plant breeder must develop superior inbred parental lines for producing hybrids by identifying and selecting genetically unique individuals that occur in a segregating population. The present disclosure contributes to this goal, for example by providing plants that, when crossed, generate male sterile progeny, which can be used as female parental plants for generating hybrid plants.

A large number of genes have been identified as being tassel preferred in their expression pattern using traditional methods and more recent high-throughput methods. The correlation of function of these genes with important biochemical or developmental processes that ultimately lead to functional pollen is arduous when approaches are limited to classical forward or reverse genetic mutational analysis. As disclosed herein, suppression approaches in maize provide an alternative rapid means to identify genes that are directly related to pollen development in maize.

Promoters useful for expressing a nucleic acid molecule of interest can be any of a range of naturally-occurring promoters known to be operative in plants or animals, as desired. Promoters that direct expression in cells of male or female reproductive organs of a plant are useful for generating a transgenic plant or breeding pair of plants of the disclosure. The promoters useful in the present disclosure can include constitutive promoters, which generally are active in most or all tissues of a plant; inducible promoters, which generally are inactive or exhibit a low basal level of expression and can be induced to a relatively high activity upon contact of cells with an appropriate inducing agent; tissue-specific (or tissue-preferred) promoters, which generally are expressed in only one or a few particular cell types (e.g., plant anther cells) and developmental- or stage-specific promoters, which are active only during a defined period during the growth or development of a plant. Often promoters can be modified, if necessary, to vary the expression level. Certain embodiments comprise promoters exogenous to the species being manipulated. For example, the Ms45 gene introduced into ms45ms45 maize germplasm may be driven by a promoter isolated from another plant species; a hairpin construct may then be designed to target the exogenous plant promoter, reducing the possibility of hairpin interaction with non-target, endogenous maize promoters.

Exemplary constitutive promoters include the 35S cauliflower mosaic virus (CaMV) promoter promoter (Odell, et al., (1985) Nature 313:810-812), the maize ubiquitin promoter (Christensen, et al., (1989) Plant Mol. Biol. 12:619-632 and Christensen, et al., (1992) Plant Mol. Biol. 18:675-689); the core promoter of the Rsyn7 promoter and other constitutive promoters disclosed in WO 1999/43838 and U.S. Pat. No. 6,072,050; rice actin (McElroy, et al., (1990) Plant Cell 2:163-171); pEMU (Last, et al., (1991) Theor. Appl. Genet. 81:581-588); MAS (Velten, et al., (1984) EMBO J. 3:2723-2730); ALS promoter (U.S. Pat. No. 5,659,026); rice actin promoter (U.S. Pat. No. 5,641,876; WO 2000/70067), maize histone promoter (Brignon, et al., (1993) Plant Mol Bio 22(6):1007-1015; Rasco-Gaunt, et al., (2003) Plant Cell Rep. 21(6):569-576) and the like. Other constitutive promoters include, for example, those described in U.S. Pat. Nos. 5,608,144 and 6,177,611 and PCT Publication Number WO 2003/102198.

Tissue-specific, tissue-preferred or stage-specific regulatory elements further include, for example, the AGL8/FRUITFULL regulatory element, which is activated upon floral induction (Hempel, et al., (1997) Development 124:3845-3853); root-specific regulatory elements such as the regulatory elements from the RCP1 gene and the LRP1 gene (Tsugeki and Fedoroff, (1999) Proc. Natl. Acad., USA 96:12941-12946; Smith and Fedoroff, (1995) Plant Cell 7:735-745); flower-specific regulatory elements such as the regulatory elements from the LEAFY gene and the APETALA1 gene (Blazquez, et al., (1997) Development 124:3835-3844; Hempel, et al., supra, 1997); seed-specific regulatory elements such as the regulatory element from the oleosin gene (Plant, et al., (1994) Plant Mol. Biol. 25:193-205) and dehiscence zone specific regulatory element. Additional tissue-specific or stage-specific regulatory elements include the Zn13 promoter, which is a pollen-specific promoter (Hamilton, et al., (1992) Plant Mol. Biol. 18:211-218); the UNUSUAL FLORAL ORGANS (UFO) promoter, which is active in apical shoot meristem; the promoter active in shoot meristems (Atanassova, et al., (1992) Plant J. 2:291), the cdc2 promoter and cyc07 promoter (see, for example, Ito, et al., (1994) Plant Mol. Biol. 24:863-878; Martinez, et al., (1992) Proc. Natl. Acad. Sci., USA 89:7360); the meristematic-preferred meri-5 and H3 promoters (Medford, et al., (1991) Plant Cell 3:359; Terada, et al., (1993) Plant J. 3:241); meristematic and phloem-preferred promoters of Myb-related genes in barley (Wissenbach, et al., (1993) Plant J. 4:411); Arabidopsis cyc3aAt and cyc1At (Shaul, et al., (1996) Proc. Natl. Acad. Sci. 93:4868-4872); C. roseus cyclins CYS and CYM (Ito, et al., (1997) Plant J. 11:983-992); and Nicotiana CyclinB1 (Trehin, et al., (1997) Plant Mol. Biol. 35:667-672); the promoter of the APETALA3 gene, which is active in floral meristems (Jack, et al., (1994) Cell 76:703; Hempel, et al., supra, 1997); a promoter of an agamous-like (AGL) family member, for example, AGL8, which is active in shoot meristem upon the transition to flowering (Hempel, et al., supra, 1997); floral abscission zone promoters; L1-specific promoters; the ripening-enhanced tomato polygalacturonase promoter (Nicholass, et al., (1995) Plant Mol. Biol. 28:423-435), the E8 promoter (Deikman, et al., (1992) Plant Physiol. 100:2013-2017) and the fruit-specific 2A1 promoter, U2 and U5 snRNA promoters from maize, the Z4 promoter from a gene encoding the Z4 22 kD zein protein, the Z10 promoter from a gene encoding a 10 kD zein protein, a Z27 promoter from a gene encoding a 27 kD zein protein, the A20 promoter from the gene encoding a 19 kD zein protein, and the like. Additional tissue-specific promoters can be isolated using well known methods (see, e.g., U.S. Pat. No. 5,589,379). Shoot-preferred promoters include shoot meristem-preferred promoters such as promoters disclosed in Weigel, et al., (1992) Cell 69:843-859 (Accession Number M91208); Accession Number AJ131822; Accession Number Z71981; Accession Number AF049870 and shoot-preferred promoters disclosed in McAvoy, et al., (2003) Acta Hort. (ISHS) 625:379-385. Inflorescence-preferred promoters include the promoter of chalcone synthase (Van der Meer, et al., (1992) Plant J. 2(4):525-535), anther-specific LAT52 (Twell, et al., (1989) Mol. Gen. Genet. 217:240-245), pollen-specific Bp4 (Albani, et al., (1990) Plant Mol Biol. 15:605, maize pollen-specific gene Zm13 (Hamilton, et al., (1992) Plant Mol. Biol. 18:211-218; Guerrero, et al., (1993) Mol. Gen. Genet. 224:161-168), microspore-specific promoters such as the apg gene promoter (Twell, et al., (1993) Sex. Plant Reprod. 6:217-224) and tapetum-specific promoters such as the TA29 gene promoter (Mariani, et al., (1990) Nature 347:737; U.S. Pat. No. 6,372,967) and other stamen-specific promoters such as the MS45 gene promoter, 5126 gene promoter, BS7 gene promoter, PG47 gene promoter (U.S. Pat. No. 5,412,085; U.S. Pat. No. 5,545,546; Plant J 3(2):261-271 (1993)), SGB6 gene promoter (U.S. Pat. No. 5,470,359), G9 gene promoter (U.S. Pat. No. 5,8937,850; U.S. Pat. No. 5,589,610), SB200 gene promoter (WO 2002/26789), or the like. Tissue-preferred promoters of interest further include a sunflower pollen-expressed gene SF3 (Baltz, et al., (1992) The Plant Journal 2:713-721), B. napus pollen specific genes (Arnoldo, et al., (1992) J. Cell. Biochem, Abstract Number Y101204). Tissue-preferred promoters further include those reported by Yamamoto, et al., (1997) Plant J. 12(2):255-265 (psaDb); Kawamata, et al., (1997) Plant Cell Physiol. 38(7):792-803 (PsPAL1); Hansen, et al., (1997) Mol. Gen Genet. 254(3):337-343 (ORF13); Russell, et al., (1997) Transgenic Res. 6(2):157-168 (waxy or ZmGBS; 27 kDa zein, ZmZ27; osAGP; osGT1); Rinehart, et al., (1996) Plant Physiol. 112(3):1331-1341 (Fbl2A from cotton); Van Camp, et al., (1996) Plant Physiol. 112(2):525-535 (Nicotiana SodA1 and SodA2); Canevascini, et al., (1996) Plant Physiol. 112(2):513-524 (Nicotiana Itp1); Yamamoto, et al., (1994) Plant Cell Physiol. 35(5):773-778 (Pinus cab-6 promoter); Lam, (1994) Results Probl. Cell Differ. 20:181-196; Orozco, et al., (1993) Plant Mol Biol. 23(6):1129-1138 (spinach rubisco activase (Rca)); Matsuoka, et al., (1993) Proc Natl. Acad. Sci. USA 90(20):9586-9590 (PPDK promoter) and Guevara-Garcia, et al., (1993) Plant J. 4(3):495-505 (Agrobacterium pmas promoter). A tissue-preferred promoter that is active in cells of male or female reproductive organs can be particularly useful in certain aspects of the present disclosure.

“Seed-preferred” promoters include both “seed-developing” promoters (those promoters active during seed development such as promoters of seed storage proteins) as well as “seed-germinating” promoters (those promoters active during seed germination). See, Thompson, et al., (1989) BioEssays 10:108. Such seed-preferred promoters include, but are not limited to, Cim1 (cytokinin-induced message), cZ19B1 (maize 19 kDa zein), mi1ps (myo-inositol-1-phosphate synthase); see, WO 2000/11177 and U.S. Pat. No. 6,225,529. Gamma-zein is an endosperm-specific promoter. Globulin-1 (Glob-1) is a representative embryo-specific promoter. For dicots, seed-specific promoters include, but are not limited to, bean 3-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, globulin 1, etc. See also, WO 2000/12733 and U.S. Pat. No. 6,528,704, where seed-preferred promoters from end1 and end2 genes are disclosed. Additional embryo specific promoters are disclosed in Sato, et al., (1996) Proc. Natl. Acad. Sci. 93:8117-8122 (rice homeobox, OSH1) and Postma-Haarsma, et al., (1999) Plant Mol. Biol. 39:257-71 (rice KNOX genes). Additional endosperm specific promoters are disclosed in Albani, et al., (1984) EMBO 3:1405-15; Albani, et al., (1999) Theor. Appl. Gen. 98:1253-62; Albani, et al., (1993) Plant J. 4:343-55; Mena, et al., (1998) The Plant Journal 116:53-62 (barley DOF); Opsahl-Ferstad, et al., (1997) Plant J 12:235-46 (maize Esr) and Wu, et al., (1998) Plant Cell Physiology 39:885-889 (rice GluA-3, GluB-1, NRP33, RAG-1).

An inducible regulatory element is one that is capable of directly or indirectly activating transcription of one or more DNA sequences or genes in response to an inducer. The inducer can be a chemical agent such as a protein, metabolite, growth regulator, herbicide or phenolic compound or a physiological stress, such as that imposed directly by heat, cold, salt or toxic elements or indirectly through the action of a pathogen or disease agent such as a virus or other biological or physical agent or environmental condition. A plant cell containing an inducible regulatory element may be exposed to an inducer by externally applying the inducer to the cell or plant such as by spraying, watering, heating or similar methods. An inducing agent useful for inducing expression from an inducible promoter is selected based on the particular inducible regulatory element. In response to exposure to an inducing agent, transcription from the inducible regulatory element generally is initiated de novo or is increased above a basal or constitutive level of expression. Typically the protein factor that binds specifically to an inducible regulatory element to activate transcription is present in an inactive form which is then directly or indirectly converted to the active form by the inducer. Any inducible promoter can be used in the instant disclosure (See, Ward, et al., (1993) Plant Mol. Biol. 22:361-366).

Examples of inducible regulatory elements include a metallothionein regulatory element, a copper-inducible regulatory element or a tetracycline-inducible regulatory element, the transcription from which can be effected in response to divalent metal ions, copper or tetracycline, respectively (Furst, et al., (1988) Cell 55:705-717; Mett, et al., (1993) Proc. Natl. Acad. Sci., USA 90:4567-4571; Gatz, et al., (1992) Plant J. 2:397-404; Roder, et al., (1994) Mol. Gen. Genet. 243:32-38). Inducible regulatory elements also include an ecdysone regulatory element or a glucocorticoid regulatory element, the transcription from which can be effected in response to ecdysone or other steroid (Christopherson, et al., (1992) Proc. Natl. Acad. Sci., USA 89:6314-6318; Schena, et al., (1991) Proc. Natl. Acad. Sci. USA 88:10421-10425; U.S. Pat. No. 6,504,082); a cold responsive regulatory element or a heat shock regulatory element, the transcription of which can be effected in response to exposure to cold or heat, respectively (Takahashi, et al., (1992) Plant Physiol. 99:383-390); the promoter of the alcohol dehydrogenase gene (Gerlach, et al., (1982) PNAS USA 79:2981-2985; Walker, et al., (1987) PNAS 84(19):6624-6628), inducible by anaerobic conditions; and the light-inducible promoter derived from the pea rbcS gene or pea psaDb gene (Yamamoto, et al., (1997) Plant J. 12(2):255-265); a light-inducible regulatory element (Feinbaum, et al., (1991) Mol. Gen. Genet. 226:449; Lam and Chua, (1990) Science 248:471; Matsuoka, et al., (1993) Proc. Natl. Acad. Sci. USA 90(20):9586-9590; Orozco, et al., (1993) Plant Mol. Bio. 23(6):1129-1138), a plant hormone inducible regulatory element (Yamaguchi-Shinozaki, et al., (1990) Plant Mol. Biol. 15:905; Kares, et al., (1990) Plant Mol. Biol. 15:225), and the like. An inducible regulatory element also can be the promoter of the maize In2-1 or In2-2 gene, which responds to benzenesulfonamide herbicide safeners (Hershey, et al., (1991) Mol. Gen. Gene. 227:229-237; Gatz, et al., (1994) Mol. Gen. Genet. 243:32-38) and the Tet repressor of transposon Tn10 (Gatz, et al., (1991) Mol. Gen. Genet. 227:229-237). Stress inducible promoters include salt/water stress-inducible promoters such as P5CS (Zang, et al., (1997) Plant Sciences 129:81-89); cold-inducible promoters, such as, cor15a (Hajela, et al., (1990) Plant Physiol. 93:1246-1252), cor15b (Wlihelm, et al., (1993) Plant Mol Biol 23:1073-1077), wsc120 (Ouellet, et al., (1998) FEBS Lett. 423:324-328), ci7 (Kirch, et al., (1997) Plant Mol Biol. 33:897-909), ci21A (Schneider, et al., (1997) Plant Physiol. 113:335-45); drought-inducible promoters, such as, Trg-31 (Chaudhary, et al., (1996) Plant Mol. Biol. 30:1247-57), rd29 (Kasuga, et al., (1999) Nature Biotechnology 18:287-291); osmotic inducible promoters, such as Rab17 (Vilardell, et al., (1991) Plant Mol. Biol. 17:985-93) and osmotin (Raghothama, et al., (1993) Plant Mol Biol 23:1117-28) and heat inducible promoters, such as heat shock proteins (Barros, et al., (1992) Plant Mol. 19:665-75; Marrs, et al., (1993) Dev. Genet. 14:27-41), smHSP (Waters, et al., (1996) J. Experimental Botany 47:325-338) and the heat-shock inducible element from the parsley ubiquitin promoter (WO 2003/102198). Other stress-inducible promoters include rip2 (U.S. Pat. No. 5,332,808 and US Patent Application Publication Number 2003/0217393) and rd29a (Yamaguchi-Shinozaki, et al., (1993) Mol. Gen. Genetics 236:331-340). Certain promoters are inducible by wounding, including the Agrobacterium pmas promoter (Guevara-Garcia, et al., (1993) Plant J. 4(3):495-505) and the Agrobacterium ORF13 promoter (Hansen, et al., (1997) Mol. Gen. Genet. 254(3):337-343).

In certain embodiments, a promoter is selected based, for example, on whether male fertility or female fertility is to be impacted Thus, where the male fertility is to be impacted, (e.g., a BS7 gene and an SB200 gene), the promoter may be, for example, an MS45 gene promoter (U.S. Pat. No. 6,037,523), a 5126 gene promoter (U.S. Pat. No. 5,837,851), a BS7 gene promoter (WO 2002/063021), an SB200 gene promoter (WO 2002/26789), a TA29 gene promoter (Nature 347:737 (1990)), a PG47 gene promoter (U.S. Pat. No. 5,412,085; U.S. Pat. No. 5,545,546; Plant J 3(2):261-271 (1993)) an SGB6 gene promoter (U.S. Pat. No. 5,470,359) a G9 gene promoter (U.S. Pat. Nos. 5,837,850 and 5,589,610) or the like. Where female fertility is to be impacted, the promoter can target female reproductive genes, for example an ovary specific promoter. In certain embodiments, any promoter can be used that directs expression in the tissue of interest, including, for example, a constitutively active promoter such as an ubiquitin promoter, which generally effects transcription in most or all plant cells.

Additional regulatory elements active in plant cells and useful in the methods or compositions of the disclosure include, for example, the spinach nitrite reductase gene regulatory element (Back, et al., (1991) Plant Mol. Biol. 17:9); a gamma zein promoter, an oleosin ole16 promoter, a globulin I promoter, an actin I promoter, an actin cI promoter, a sucrose synthetase promoter, an INOPS promoter, an EXM5 promoter, a globulin2 promoter, a b-32, ADPG-pyrophosphorylase promoter, an Ltp1 promoter, an Ltp2 promoter, an oleosin ole17 promoter, an oleosin ole18 promoter, an actin 2 promoter, a pollen-specific protein promoter, a pollen-specific pectate lyase gene promoter or PG47 gene promoter, an anther specific RTS2 gene promoter, SGB6 gene promoter or G9 gene promoter, a tapetum specific RAB24 gene promoter, an anthranilate synthase alpha subunit promoter, an alpha zein promoter, an anthranilate synthase beta subunit promoter, a dihydrodipicolinate synthase promoter, a Thi I promoter, an alcohol dehydrogenase promoter, a cab binding protein promoter, an H3C4 promoter, a RUBISCO SS starch branching enzyme promoter, an actin3 promoter, an actin7 promoter, a regulatory protein GF14-12 promoter, a ribosomal protein L9 promoter, a cellulose biosynthetic enzyme promoter, an S-adenosyl-L-homocysteine hydrolase promoter, a superoxide dismutase promoter, a C-kinase receptor promoter, a phosphoglycerate mutase promoter, a root-specific RCc3 mRNA promoter, a glucose-6 phosphate isomerase promoter, a pyrophosphate-fructose 6-phosphate-l-phosphotransferase promoter, a beta-ketoacyl-ACP synthase promoter, a 33 kDa photosystem 11 promoter, an oxygen evolving protein promoter, a 69 kDa vacuolar ATPase subunit promoter, a glyceraldehyde-3-phosphate dehydrogenase promoter, an ABA- and ripening-inducible-like protein promoter, a phenylalanine ammonia lyase promoter, an adenosine triphosphatase S-adenosyl-L-homocysteine hydrolase promoter, a chalcone synthase promoter, a zein promoter, a globulin-1 promoter, an auxin-binding protein promoter, a UDP glucose flavonoid glycosyl-transferase gene promoter, an NTI promoter, an actin promoter and an opaque 2 promoter.

Plants suitable for purposes of the present disclosure can be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis thaliana and woody plants such as coniferous and deciduous trees. Thus, a transgenic plant or genetically modified plant cell of the disclosure can be an angiosperm or gymnosperm.

Angiosperms are divided into two broad classes based on the number of cotyledons, which are seed leaves that generally store or absorb food; a monocotyledonous angiosperm has a single cotyledon and a dicotyledonous angiosperm has two cotyledons. Angiosperms produce a variety of useful products including materials such as lumber, rubber and paper; fibers such as cotton and linen; herbs and medicines such as quinine and vinblastine; ornamental flowers such as roses and where included within the scope of the present disclosure, orchids and foodstuffs such as grains, oils, fruits and vegetables. Angiosperms encompass a variety of flowering plants, including, for example, cereal plants, leguminous plants, oilseed plants, hardwood trees, fruit-bearing plants and ornamental flowers, which general classes are not necessarily exclusive. Cereal plants, which produce an edible grain, include, for example, corn, rice, wheat, barley, oat, rye, orchardgrass, guinea grass and sorghum. Leguminous plants include members of the pea family (Fabaceae) and produce a characteristic fruit known as a legume. Examples of leguminous plants include, for example, soybean, pea, chickpea, moth bean, broad bean, kidney bean, lima bean, lentil, cowpea, dry bean and peanut, as well as alfalfa, birdsfoot trefoil, clover and sainfoin. Oilseed plants, which have seeds that are useful as a source of oil, include soybean, sunflower, rapeseed (canola) and cottonseed. Angiosperms also include hardwood trees, which are perennial woody plants that generally have a single stem (trunk). Examples of such trees include alder, ash, aspen, basswood (linden), beech, birch, cherry, cottonwood, elm, eucalyptus, hickory, locust, maple, oak, persimmon, poplar, sycamore, walnut, sequoia and willow. Trees are useful, for example, as a source of pulp, paper, structural material and fuel.

Angiosperms produce seeds enclosed within a mature, ripened ovary. An angiosperm fruit can be suitable for human or animal consumption or for collection of seeds to propagate the species. For example, hops are a member of the mulberry family that are prized for their flavoring in malt liquor. Fruit-bearing angiosperms also include grape, orange, lemon, grapefruit, avocado, date, peach, cherry, olive, plum, coconut, apple and pear trees and blackberry, blueberry, raspberry, strawberry, pineapple, tomato, cucumber and eggplant plants. An ornamental flower is an angiosperm cultivated for its decorative flower. Examples of commercially important ornamental flowers include rose, lily, tulip and chrysanthemum, snapdragon, camellia, carnation and petunia plants and can include orchids. It will be recognized that the present disclosure also can be practiced using gymnosperms, which do not produce seeds in a fruit.

Homozygosity is a genetic condition existing when identical alleles reside at corresponding loci on homologous chromosomes. Heterozygosity is a genetic condition existing when different alleles reside at corresponding loci on homologous chromosomes. Hemizygosity is a genetic condition existing when there is only one copy of a gene (or set of genes) with no allelic counterpart on the sister chromosome.

The plant breeding methods used herein are well known to one skilled in the art. For a discussion of plant breeding techniques, see, Poehlman, (1987) Breeding Field Crops AVI Publication Co., Westport Conn. Many of the plants which would be most preferred in this method are bred through techniques that take advantage of the plant's method of pollination.

Backcrossing methods may be used to introduce a gene into the plants. This technique has been used for decades to introduce traits into a plant. An example of a description of this and other plant breeding methodologies that are well known can be found in references such as Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

By transgene is meant any nucleic acid sequence which has been introduced into the genome of a cell by genetic engineering techniques. A transgene may be a native DNA sequence or a heterologous DNA sequence. The term native DNA sequence can refer to a nucleotide sequence which is naturally found in the cell but that may have been modified from its original form.

Using well-known techniques, additional promoter sequences may be isolated based on their sequence homology. In these techniques, all or part of a known promoter sequence is used as a probe which selectively hybridizes to other sequences present in a population of cloned genomic DNA fragments (i.e., genomic libraries) from a chosen organism. Methods that are readily available in the art for the hybridization of nucleic acid sequences may be used to obtain sequences which correspond to these promoter sequences in species including, but not limited to, maize (corn; Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats, barley, vegetables, ornamentals and conifers. Preferably, plants include maize, soybean, sunflower, safflower, canola, wheat, barley, rye, alfalfa and sorghum.

The entire promoter sequence or portions thereof can be used as a probe capable of specifically hybridizing to corresponding promoter sequences. To achieve specific hybridization under a variety of conditions, such probes include sequences that are unique and are preferably at least about 10 nucleotides in length and most preferably at least about 20 nucleotides in length. Such probes can be used to amplify corresponding promoter sequences from a chosen organism by the well-known process of polymerase chain reaction (PCR). This technique can be used to isolate additional promoter sequences from a desired organism or as a diagnostic assay to determine the presence of the promoter sequence in an organism. Examples include hybridization screening of plated DNA libraries (either plaques or colonies; see e.g., Innis, et al., (1990) PCR Protocols, A Guide to Methods and Applications, eds., Academic Press).

In general, sequences that correspond to a promoter sequence of the present disclosure and hybridize to a promoter sequence disclosed herein will be at least 50% homologous, 55% homologous, 60% homologous, 65% homologous, 70% homologous, 75% homologous, 80% homologous, 85% homologous, 90% homologous, 95% homologous and even 98% homologous or more with the disclosed sequence.

Fragments of a particular promoter sequence disclosed herein may operate to promote the pollen-preferred expression of an operably-linked isolated nucleotide sequence. These fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequences disclosed herein. The nucleotides of such fragments will usually comprise the TATA recognition sequence of the particular promoter sequence. Such fragments can be obtained by use of restriction enzymes to cleave the naturally-occurring promoter sequences disclosed herein; by synthesizing a nucleotide sequence from the naturally-occurring DNA sequence or through the use of PCR technology. See particularly, Mullis, et al., (1987) Methods Enzymol. 155:335-350 and Erlich, ed. (1989) PCR Technology (Stockton Press, New York). Again, variants of these fragments, such as those resulting from site-directed mutagenesis, are encompassed by the compositions of the present disclosure.

Biologically active variants of the promoter sequence are also encompassed by the compositions of the present disclosure. A regulatory “variant” is a modified form of a promoter wherein one or more bases have been modified, removed or added. For example, a routine way to remove part of a DNA sequence is to use an exonuclease in combination with DNA amplification to produce unidirectional nested deletions of double-stranded DNA clones. A commercial kit for this purpose is sold under the trade name Exo-Size™ (New England Biolabs, Beverly, Mass.). Briefly, this procedure entails incubating exonuclease III with DNA to progressively remove nucleotides in the 3′ to 5′ direction at 5′ overhangs, blunt ends or nicks in the DNA template. However, exonuclease III is unable to remove nucleotides at 3′, 4-base overhangs. Timed digests of a clone with this enzyme produce unidirectional nested deletions.

One example of a regulatory sequence variant is a promoter formed by causing one or more deletions in a larger promoter. Deletion of the 5′ portion of a promoter up to the TATA box near the transcription start site may be accomplished without abolishing promoter activity, as described by Zhu, et al., (1995) The Plant Cell 7:1681-89. Such variants should retain promoter activity, particularly the ability to drive expression in specific tissues. Biologically active variants include, for example, the native regulatory sequences of the disclosure having one or more nucleotide substitutions, deletions or insertions. Activity can be measured by Northern blot analysis, reporter activity measurements when using transcriptional fusions, and the like. See, for example, Sambrook, et al., (1989) Molecular Cloning: A Laboratory Manual (2nd ed. Cold Spring Harbor Laboratory, Cold Spring Harbor, N.Y.), herein incorporated by reference.

The nucleotide sequences for the pollen-preferred promoters disclosed in the present disclosure, as well as variants and fragments thereof, are useful in the genetic manipulation of any plant when operably linked with an isolated nucleotide sequence whose expression is to be controlled to achieve a desired phenotypic response.

The nucleotide sequence operably linked to the regulatory elements disclosed herein can be an antisense sequence for a targeted gene. By “antisense DNA nucleotide sequence” is intended a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing with the endogenous messenger RNA (mRNA) produced by transcription of the DNA nucleotide sequence for the targeted gene. In this case, production of the native protein encoded by the targeted gene is inhibited to achieve a desired phenotypic response. Thus the regulatory sequences claimed herein can be operably linked to antisense DNA sequences to reduce or inhibit expression of a native or exogenous protein in the plant.

Regulation of gene expression may be measured in terms of its effect on individual cells. Successful modulation of a trait may be accomplished with high stringency, for example impacting expression in all or nearly all cells of a particular cell type, or with lower stringency. Within a particular tissue, for example, modulation of expression in 98%, 95%, 90%, 80% or fewer cells may result in the desired phenotype.

### EXAMPLES

### Example 1

### Identification and Isolation of ACO Genes

Bioinformatic search tools were used to identify polynucleotides or polypeptides with common sequences or sequence elements. Four ZmACOs (SEQ ID NO: 4, 8, 10, 20) were used to search maize databases for any additional ZmACO sequences. Six additional ZmACOs were identified (SEQ ID NO: 2, 6, 12, 14, 16, 18). FIG. 1 shows a phylogenetic tree that was created to compare the ten ZmACOs. ZmACO6 and ZmACO9 appear to be more distinct in their origin, while the other ZmACOs fall into two separate groups.

### Example 2

### ACO2 RNAi Construct (PHP583) and Results

The objective of this research was to use a transgenic approach to reduce the synthesis of ethylene in maize to permit growth under drought stress and lead to an increase in grain yield. This goal was accomplished by silencing the expression of ACC oxidase (ACO) via an ACC oxidase 2 (ACO2) hairpin construct.

A hairpin construct was designed and built to silence the expression of ACO2. The plasmid was generated by linking an ubiquitin promoter to inverted repeats which contained a fragment of the ACO2 sequence (SEQ ID NO: 41) that targets the ACO2 gene for down regulation. The construct included an ADH1 intron spacer segment between the inverted repeat sequences. PHP583 was introduced into maize via Agrobacterium-mediated transformation using methods known in the art and referenced elsewhere herein. FIG. 2 demonstrates that an RNAi construct targeting ACO2 effectively knocked down endogenous ACO2 transcript levels relative to the control.

Transgenic hybrid events transformed with UBI:ZM-ACO2 RNAi showed improved yield under drought conditions in field yield trials. The effect of silencing ACO2 in transgenic maize hybrids was evaluated in field yield trials. Multiple events were created by independent transformation of a maize line with PHP583. Transgenic lines from eight independent events were top-crossed to an appropriate tester. The transgenic hybrids were tested in both managed drought stress and normal Corn-Belt locations. The grain yield of transgenic events was evaluated against a bulk null comparator. Multi-location statistical analysis indicated that 4 out of the 8 events had a statistically significant (P<0.1) grain yield increase relative to the comparator. A significant increase in yield was determined for the four events at a managed drought stress location with no significant yield penalty measured at normal Corn-Belt sites.

This Example demonstrates that the down regulation of an ACC oxidase gene in a crop plant resulted in a significant increase in grain yield of the crop plant under drought conditions and no significant yield penalty under normal water conditions.

### Example 3

### ACO2-ACO5-ACO6 RNAi Stack Construct (PHP666) and Results

A hairpin construct was designed and built to silence the expression of several ACC oxidases. The plasmid was generated by linking an ubiquitin promoter to inverted repeats which contained individual fragments of ACO2, ACO5 and ACO6 (SEQ ID NO: 41, 43, 42; respectively), including an ADH1 intron spacer segment between the inverted repeat sequences. PHP666 was introduced into maize via Agrobacterium-mediated transformation using methods known in the art and referenced elsewhere herein. FIG. 3 shows that the RNAi construct targeting ACO2, ACO5, and ACO6 effectively knocked down endogenous transcript levels of all genes relative to the control.

One of the objectives of this research was to use a transgenic approach to reduce the synthesis of ethylene in maize to permit growth under drought stress and lead to an increase in grain yield. An approach undertaken was to reduce the expression of ACC oxidases via an ACC oxidase 2/5/6 hairpin construct. The down-regulation elements were expressed in maize via a constitutive Ubiquitin promoter. Transgenic hybrid events transformed with the ZM-ACO2 (TR1)/ZM-ACO5 (TR1)/ZM-ACO6 (TR1) RNAi construct showed improved yield under drought conditions in field yield trials.

The effect of reducing multiple (ACO2, ACO5, ACO6) ACC oxidases in transgenic maize hybrids was evaluated in field yield trials. Multiple events were created by independent transformation of a maize line with PHP666. Transgenic lines from seven independent events were top-crossed to an appropriate tester. The transgenic hybrids were tested in both managed drought stress and normal Corn-Belt locations. The grain yield of transgenic events was evaluated against a bulk null comparator. Multi-location statistical analysis indicated that 4 out of the 7 events had a statistically significant (P<0.1) grain yield increase relative to the comparator and there was no yield penalty at any of the locations.

This Example demonstrates that the down regulation of a combination of ACC oxidase genes in a crop plant resulted in a significant increase in grain yield of the crop plant under drought conditions and no significant yield penalty under normal water conditions.

We claim:

1. A method of improving abiotic stress tolerance in a crop plant, the method comprising reducing the expression of an ACC oxidase gene in the crop plant and growing the crop plant in a plant growing environment, wherein the crop plant is exposed to an abiotic stress.

2. The method of claim 1, wherein the abiotic stress is drought stress.

3. The method of claim 1, wherein the ACC oxidase gene expression that is reduced comprises a polynucleotide encoding a polypeptide selected from the group consisting of SEQ ID NOS: 21-30, 59, 61, 63, 65, 67, 69 and 71 or an amino acid sequence that is at least 95% identical to the polypeptide thereof.

4. The method of claim 1, wherein the ACC oxidase gene that is down regulated comprises a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

5. The method of claim 1, wherein the ACC oxidase gene is down regulated by a RNA-interference construct that comprises a nucleic acid element that targets an endogenous mRNA sequence transcribed from a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

6. The method of claim 1, wherein the ACC oxidase gene comprises a polynucleotide selected from the group consisting of SEQ ID NOS: 1-20, 31-40, 58, 60, 62, 64, 66, 68 and 70 or a nucleotide sequence that is at least 95% identical to the polynucleotide thereof and wherein the ACC oxidase gene is down regulated by a genetic modification.

7. An abiotic stress tolerant transgenic maize plant comprising in its genome a recombinant nucleic acid that down regulates the expression of an endogenous ACO gene, wherein the ACO gene comprises a polynucleotide that encodes a polypeptide selected from the group consisting of SEQ ID NOS: 21-30.

8. The maize plant of claim 7, wherein the abiotic stress is drought, low nitrogen, heat or salt.

9. The maize plant of claim 7, wherein the recombinant nucleic acid down regulates the expression of ACO2, ACO5 and ACO6.

10. The maize plant of claim 9, wherein the recombinant nucleic acid sequences comprises a polynucleotide sequence selected from the group consisting of SEQ ID NOS: 41-43.

11. A plant cell produced from the maize plant of claim 7.

12. A seed produced from the maize plant of claim 7.

13. A method of increasing grain yield of a crop plant under drought conditions, the method comprising reducing the levels of ethylene in the crop plant, wherein the reduction in ethylene levels are not accompanied by a reduction in ACC levels within the crop plant and growing the crop plant in a crop growing condition, wherein the crop plant is exposed to drought stress and thereby increasing the grain yield of the crop plant.

14. The method of claim 13, wherein the crop plant is maize.

15. The method of claim 13, wherein the ethylene levels are reduced by the down regulation of a gene encoding an ACC oxidase.

16. The method of claim 15, wherein the ACC oxidase gene that is down regulated comprises a polynucleotide encoding a polypeptide selected from the group consisting of SEQ ID NOS: 21-30, 59, 61, 63, 65, 67, 69 and 71 or an amino acid sequence that is at least 95% identical to the polypeptide thereof.

17. The method of claim 15, wherein the ACC oxidase gene that is down regulated comprises a polynucleotide selected from the group consisting of SEQ ID NOS: 1-20, 31-40, 58, 60, 62, 64, 66, 68 and 70 or a nucleotide sequence that is at least 95% identical to the polynucleotide thereof.

18. A gene down regulation construct comprising an isolated nucleic acid that is transcribed into a plurality of interfering RNA transcripts, wherein the interfering RNA transcripts reduce the expression of a plurality of polynucleotide sequences that encode a plurality of polypeptides selected from the group consisting of SEQ ID NOS: 21-30, 59, 61, 63, 65, 67, 69 and 71 or an amino acid sequence that is at least 95% identical to the polypeptide thereof.

19. The construct of claim 18 wherein the construct is a hairpin construct.

20. A vector comprising the construct of claim 18.

21. A method of down regulation of an endogenous ACC oxidase gene in a maize plant, the method comprising expressing a recombinant nucleic acid construct that reduces the expression of the endogenous ACC oxidase gene selected from the group consisting of SEQ ID NOS: 1-20 or an allelic variant of the sequences thereof.

22. The method of claim 21, wherein the expression of the endogenous ACC oxidase gene is reduced by a recombinant construct comprising a polynucleotide sequence selected from the group consisting of SEQ ID NOS: 41-43.

23. The method of claim 21, wherein the ACC oxidase gene that is being down regulated is selected from the group consisting of SEQ ID NOS: 3-6, 11-12, 32-33, 36 and 39 or a nucleotide sequence that is an allelic variant of SEQ ID NOS: 3-6, 11-12, 32-33, 36 and 39.

24. The method of claim 21, wherein the ACC oxidase gene is ACO2.

25. The method of claim 21, wherein ACC oxidase gene comprises a polynucleotide encoding a polypeptide selected from the group consisting of SEQ ID NOS: 22 and 23.

26. The method of claim 1, wherein the crop plant is monocot.

27. A method of selecting a maize plant from a population of maize plants for increased drought tolerance, the method comprising screening a population of plants for a reduced expression of an ACO gene selected from the group consisting of SEQ ID NOS: 1-20 or an allelic variant of the sequences thereof.

28. The method of claim 27, wherein the maize population is an inbred population.


SEQUENCE LISTING (71 sequences)

SEQ ID NO: 1 - DNA, 1501 bp, Zea mays
gcgttcagcattagacacgagagctcctagtagccagaccagtagtcccgcgaccctgtcgagagaaacagacagagcaacatggcgcctgcattgtcattcccgatcatcgacatggggctgctcgccggggaggagaggccggcggcgatggagctgctgcaagatgcgtgcgagaactggggcttcttcgagattctgaaccacggcatctcgacggagctgatggacgaggtagagaagctgaccaaggagcactacaagcgggtgcgcgagcagaggttcctcgagttcgccagcaagacgctcggggacggccgcgacattgcgcagggcgtgaaggcggagaacctggactgggagagcaccttcttcgtccgccacctcccggagcccaacatcgccgagataccggacctggacgacgagtaccggcgcgtcatgaagcggttcgccggcgagctggaggcgctggcggagcggctgctggacctgctgtgcgagaacctcggcctcgacaggggctacctggcgcgcgcgttccgcgggcccagcaagggcgccccgacgttcggcaccaaggtcagcagctacccgccgtgcccgcgcccggacctcgtcagcggcctgcgcgcgcacaccgacgccggcggcatcatcctgctgttccaggacgaccgggtgggcggcctccagctgctcaaggacggcgagtgggttgacgtgccgcccatgcgccacgccgtcgtcgtcaacctgggcgaccagctggaggtgatcaccaacggcaggtacaagagcgtcatgcaccgggtggtggcgcagcccagcgggaacaggatgtccatcgcgtccttctacaacccgggcagcgacgcggtcatcttcccggcgccggcgctggtcaaggccgaggaggcggcagcgggggcgtaccccagcttcgtcttcgaggactacatgaagctgtacgtgcggcacaagttcgaggccaaggagccacggttcgaggccttcaagtccatggagacggacagctccaatcgcatagccatcgcgtgaaacaccggacctgcgccgagctctggcttactgttcgagatgtacgtgcggcgtactgtactcactaccggaatccgagactttgccgagtgttggcttctttgccgagtgccttttgtcgggcactcggcaaagaaagctttgccgagtgccgtactcggtaacgttaggcactcggcaaaacgtgctttgccgagggctgaacactcggcacagaacggcactcggcaaagacaactttgccgagagtcaaacactcggcaaaggaggctctcggcgagcggccgtcccaaagctgacggccgttagtctttgccgagtgtcatccgttggctctcggcaaagaggttctgtaccgagtgccacatagtaggcactcggcaaagcatactttgccgagtgtcatctctggacactcggcaaagtatatttttatttt

SEQ ID NO: 2 - DNA, 978 bp, Zea mays
atggcgcctgcattgtcattcccgatcatcgacatggggctgctcgccggggaggagaggccggcggcgatggagctgctgcaagatgcgtgcgagaactggggcttcttcgagattctgaaccacggcatctcgacggagctgatggacgaggtagagaagctgaccaaggagcactacaagcgggtgcgcgagcagaggttcctcgagttcgccagcaagacgctcggggacggccgcgacattgcgcagggcgtgaaggcggagaacctggactgggagagcaccttcttcgtccgccacctcccggagcccaacatcgccgagataccggacctggacgacgagtaccggcgcgtcatgaagcggttcgccggcgagctggaggcgctggcggagcggctgctggacctgctgtgcgagaacctcggcctcgacaggggctacctggcgcgcgcgttccgcgggcccagcaagggcgccccgacgttcggcaccaaggtcagcagctacccgccgtgcccgcgcccggacctcgtcagcggcctgcgcgcgcacaccgacgccggcggcatcatcctgctgttccaggacgaccgggtgggcggcctccagctgctcaaggacggcgagtgggttgacgtgccgcccatgcgccacgccgtcgtcgtcaacctgggcgaccagctggaggtgatcaccaacggcaggtacaagagcgtcatgcaccgggtggtggcgcagcccagcgggaacaggatgtccatcgcgtccttctacaacccgggcagcgacgcggtcatcttcccggcgccggcgctggtcaaggccgaggaggcggcagcgggggcgtaccccagcttcgtcttcgaggactacatgaagctgtacgtgcggcacaagttcgaggccaaggagccacggttcgaggccttcaagtccatggagacggacagctccaatcgcatagccatcgcg

SEQ ID NO: 3 - DNA, 1304 bp, Zea mays
caagcctgccctgtcctgccttgttaagcaacacagcgagacatcacgagagctagagagagatggcggccacggtttcctccttcccggtggtgaacatggagaagctggagacagaggagagggccacggccatggaggtcatccgcgacggctgcgagaactggggcttcttcgagctgctgaaccacggcatctcgcacgagctgatggacgaggtggagcggctgaccaaggcgcactacgccaccttccgggaggccaagttccaggagttcgcggcgcggacgctggaggccggcgagaagggcgccgacgtcaaggacgtggactgggagagcaccttcttcgtccgccacctcccggcctccaacctcgccgacctccccgacgtcgacgaccgctacaggcaggtgatggagcagttcgcatcggagatccggaagctgtcggagaggctgctggacctgctgtgcgagaacctgggcctggagcccgggtacctgaaggcggccttcgcggggtcggacggcccgacgttcggcaccaaggtgagcgcgtacccgccgtgcccgcgcccggacctcgtcgacggcctccgcgcgcacaccgacgccggcggcatcgtgctgctgttccaggacgaccaggtgagcggcctgcagctgctcaggggcggggagtgggtggacgtgccgcccatgcgccacgccatcgtcgccaacgtcggcgaccagctggaggtcatcaccaacgggcggtacaagagcgtcatgcaccgcgtgctcacgcgccccgacggcaaccgcatgtccgtcgcgtccttctacaacccgggcgccgacgccgtcatcttcccggcgcccgcgctcgtcggcgccgccgaggaggaccgcgccgaggccgcgtacccgagcttcgtgttcgaggactacatgaacctgtacgtgcgccacaagttcgaggccaaggagcccaggttcgaggccatgaagtcggccatcgccaccgcgtgagaaagactgccttccgctgccggcttccttcgtggcgtcaagccttgaggcttgaacgaacaacgtacgtccatgtgcttatagtggcacagttgtgtgtgtaactaccgatcgtggaacggcctaatgtatttcggttgcctcagatcgatctatatgtgcgtatacattatgtactcaaaagtgtgtagcgtctggttaatgtacgagcagtgtgtatgtgaccaggacccggtgtgtagttgctattactaccatatccggtgaatgatcaaaccttttggtgta

SEQ ID NO: 4 - DNA, 951 bp, Zea mays
atggcggccacggtttcctccttcccggtggtgaacatggagaagctggagacagaggagagggccacggccatggaggtcatccgcgacggctgcgagaactggggcttcttcgagctgctgaaccacggcatctcgcacgagctgatggacgaggtggagcggctgaccaaggcgcactacgccaccttccgggaggccaagttccaggagttcgcggcgcggacgctggaggccggcgagaagggcgccgacgtcaaggacgtggactgggagagcaccttcttcgtccgccacctcccggcctccaacctcgccgacctccccgacgtcgacgaccgctacaggcaggtgatggagcagttcgcatcggagatccggaagctgtcggagaggctgctggacctgctgtgcgagaacctgggcctggagcccgggtacctgaaggcggccttcgcggggtcggacggcccgacgttcggcaccaaggtgagcgcgtacccgccgtgcccgcgcccggacctcgtcgacggcctccgcgcgcacaccgacgccggcggcatcgtgctgctgttccaggacgaccaggtgagcggcctgcagctgctcaggggcggggagtgggtggacgtgccgcccatgcgccacgccatcgtcgccaacgtcggcgaccagctggaggtcatcaccaacgggcggtacaagagcgtcatgcaccgcgtgctcacgcgccccgacggcaaccgcatgtccgtcgcgtccttctacaacccgggcgccgacgccgtcatcttcccggcgcccgcgctcgtcggcgccgccgaggaggaccgcgccgaggccgcgtacccgagcttcgtgttcgaggactacatgaacctgtacgtgcgccacaagttcgaggccaaggagcccaggttcgaggccatgaagtcggccatcgccaccgcg

SEQ ID NO: 5 - DNA, 1320 bp, Zea mays
caaactcaagcctgccctgccctgccttgttaagcaaagcaacccagctgcgagacacgagagctagctagagagagatggcggccacggtttcctccttcccggtggtgaacatggagaagctggagacagaggagagggccacggccatggaggtcatccgcgacggctgcgagaactggggcttcttcgagctgctgaaccacggcatctcgcacgagctgatggacgaggtggagcggctgaccaaggcgcactacgccaccttccgggaggccaagttccaggagttcgcggcccggacgctggaggccggcgagaagggcgccgacgtcaaggacgtggactgggagagcaccttcttcgtccgccacctcccggcctccaacctcgccgacctccccgacgtcgacgaccgctacaggcaggtgatggagcagttcgcatcggagatccgcaagctgtcggagaggctgctggacctgctgtgcgagaacctgggcctggagcccgggtacctgaaggcggccttcgcggggtcggacggcccgacgttcggcaccaaggtgagcgcgtacccgccgtgcccgcgcccggacctcgtcgacggcctccgcgcgcacaccgacgccggcggcatcgtgctgctgttccaggacgaccaggtgagcggcctgcagctgctcaggggcggggagtgggtggacgtgccgcccatgcgccacgccatcgtcgccaacgtcggcgaccagctggaggtgatcaccaacgggcggtacaagagcgtcatgcaccgcgtgctcacgcgccccgacggcaaccgcatgtccgtcgcgtccttctacaacccgggcgccgacgccgtcatcttcccggcccccgcgctcgtcggcgccgccgaggaggaccgcgccgaggccgcgtacccgagcttcgtgttcgaggactacatgaacctgtacgtgcgccacaagttcgaggccaaggagcccaggttcgaggccatgaagtcggccatcgccaccgcgtgagagaagactgccttccgctgcaggcttccttcgtggcgtcaagccttgaggcttgaacgaacaacgtacgtccatgtgcttatagtggcacagttatgtgtgtaactaccgatcgtggaacggcctaatgtatttcggttgcctcagatcgatctatatgtgcgtatacattatgtactgaaaagtgtgtagcgtctggttaatgtatgagcagtgtgtatgtgaccgggacccggtgtgtagttgctattactaccatatccggtgaatgatcaaaccttttggtgta

SEQ ID NO: 6 - DNA, 951 bp, Zea mays
atggcggccacggtttcctccttcccggtggtgaacatggagaagctggagacagaggagagggccacggccatggaggtcatccgcgacggctgcgagaactggggcttcttcgagctgctgaaccacggcatctcgcacgagctgatggacgaggtggagcggctgaccaaggcgcactacgccaccttccgggaggccaagttccaggagttcgcggcccggacgctggaggccggcgagaagggcgccgacgtcaaggacgtggactgggagagcaccttcttcgtccgccacctcccggcctccaacctcgccgacctccccgacgtcgacgaccgctacaggcaggtgatggagcagttcgcatcggagatccgcaagctgtcggagaggctgctggacctgctgtgcgagaacctgggcctggagcccgggtacctgaaggcggccttcgcggggtcggacggcccgacgttcggcaccaaggtgagcgcgtacccgccgtgcccgcgcccggacctcgtcgacggcctccgcgcgcacaccgacgccggcggcatcgtgctgctgttccaggacgaccaggtgagcggcctgcagctgctcaggggcggggagtgggtggacgtgccgcccatgcgccacgccatcgtcgccaacgtcggcgaccagctggaggtgatcaccaacgggcggtacaagagcgtcatgcaccgcgtgctcacgcgccccgacggcaaccgcatgtccgtcgcgtccttctacaacccgggcgccgacgccgtcatcttcccggcccccgcgctcgtcggcgccgccgaggaggaccgcgccgaggccgcgtacccgagcttcgtgttcgaggactacatgaacctgtacgtgcgccacaagttcgaggccaaggagcccaggttcgaggccatgaagtcggccatcgccaccgcg

SEQ ID NO: 7 - DNA, 1252 bp, Zea mays
ggcctgcctgttaagcaacccggcgagcgaggtggtgagagaacgagcgagagggagatggcagccacggtgtccttcccggtggtgaacatggagaagctggagaccgaggagagggacacggccatggcggtcatccgcgacgcctgcgagaactggggcttcttcgagctgctgaaccatggcatctcgcacgagctgatggacgaggtggagcggctgaccaaggcgcactacgccaccttccgggaggccaagttccaggagttcgcggcgcggacgctggccgcggccggcgacgagggcgccgacgtcagcgacgtggactgggagagcaccttcttcgtccgccacctcccggcctccaacctcgccgacctccccgacgtcgacgaccactaccggcaagtgatgaagcagttcgcatcggaggtgcagaagctgtcggagaaggtgctggacctgctgtgcgagaacctgggcctggagcccgggtacctgaaggcggccttcgcggggtcggacggcggcccgacgttcggcaccaaggtgagcgcgtacccgccgtgcccgcgcccggacctggtggccggcctgcgcgcgcacaccgacgccggcggcctcatcctgctgctccaggacgaccaggtgagcgggctgcagctgctcaggggcggcgacggcggggagtgggtggacgtgccgccgctgcgccacgccatcgtcgccaacgtcggcgaccagctggaggtggtcaccaacgggcggtacaagagcgcggtgcaccgcgtgctcgcccgccccgacggcaaccgcatgtccgtcgcgtccttctacaacccgggcgccgacgccgtcatcttcccggcccccgcgctcgtcggcgaggaggagcgagccgagaagaaggccaccacgtacccgaggttcgtgttcgaggactacatgaacctgtacgcgcgccacaagttcgaggccaaggagccccggttcgaggccatgaagtcgtcggccatcgccaccgcgtgagcacataatactgccgtgttctcccttcgtggggtgcatatgcttgagcttgaagagccatgtgcctgtatgtagtggcacgtacggtggttatgcgtgtatcgtggaatggcgcggcgtgatgtattttggttgtctcagatctaagtgtgtgcgtatatattgtgtactgtaaagtttgcagcgtctgattaatgtacgagcagtgtgtgtacctaaccag

SEQ ID NO: 8 - DNA, 969 bp, Zea mays
atggcagccacggtgtccttcccggtggtgaacatggagaagctggagaccgaggagagggacacggccatggcggtcatccgcgacgcctgcgagaactggggcttcttcgagctgctgaaccatggcatctcgcacgagctgatggacgaggtggagcggctgaccaaggcgcactacgccaccttccgggaggccaagttccaggagttcgcggcgcggacgctggccgcggccggcgacgagggcgccgacgtcagcgacgtggactgggagagcaccttcttcgtccgccacctcccggcctccaacctcgccgacctccccgacgtcgacgaccactaccggcaagtgatgaagcagttcgcatcggaggtgcagaagctgtcggagaaggtgctggacctgctgtgcgagaacctgggcctggagcccgggtacctgaaggcggccttcgcggggtcggacggcggcccgacgttcggcaccaaggtgagcgcgtacccgccgtgcccgcgcccggacctggtggccggcctgcgcgcgcacaccgacgccggcggcctcatcctgctgctccaggacgaccaggtgagcgggctgcagctgctcaggggcggcgacggcggggagtgggtggacgtgccgccgctgcgccacgccatcgtcgccaacgtcggcgaccagctggaggtggtcaccaacgggcggtacaagagcgcggtgcaccgcgtgctcgcccgccccgacggcaaccgcatgtccgtcgcgtccttctacaacccgggcgccgacgccgtcatcttcccggcccccgcgctcgtcggcgaggaggagcgagccgagaagaaggccaccacgtacccgaggttcgtgttcgaggactacatgaacctgtacgcgcgccacaagttcgaggccaaggagccccggttcgaggccatgaagtcgtcggccatcgccaccgcg

SEQ ID NO: 9 - DNA, 1237 bp, Zea mays
gctagctagccttccctacagcaactgcatacatacaacacttccatctgcccgctcgtcttcgatcaattcccaagtcaaataataatataacagcaatggtggttcccgtgatcgacttctccaagctggacggcgctgagagggctgaaaccctggcgcagatcgccaatggctgcgaggagtggggattcttccagctcgtgaaccacggcatcccgctggagctgctcgagcgcgtcaagaaggtgtgctccgactgctaccgcctccgggaggccgggttcaaggcgtcggagccggtgcgcacgctggaggcgctcgtcgacgcggagcggcgcggtgaggtggtggcgccggtggacgacctggactgggaggacatcttctacatccacgacggatgccagtggccgtccgacccgccggcgttcaaggagaccatgcgcgagtaccgcgccgagctgaggaagctcgccgagcgagtcatggaggccatggacgagaacctcggcctcgccaggggcaccatcaaggacgccttctccggcggcggccggcacgatcccttcttcggcaccaaggtcagccactacccgccgtgcccacgcccggacctcatcacgggcctgcgcgcgcacaccgacgccggcggcgtcatcctcctgttccaggacgacaaggtcggtggcctggaggtgctcaaggacggcgagtggaccgacgtacagccgctcgagggcgccatcgtcgtcaacaccggcgaccagatcgaggtgctcagcaacgggctgtaccgcagcgcttggcaccgcgtgctgcccatgcgcgacggcaatcgccgctccatcgcatccttctacaacccagccaacgaagccaccatctcgccggcggcggtgcaggccagcggcggtgacgcgtatcccaagtacttgttcggcgattacatggacgtgtacgtcaagcagaagttccaggccaaggagcctaggttcgaagccgtcaagacgggggcgccaaagtcatctccagcggcataaataaacagggaaaacaattattgaatgcattattaaaaggtagtaataagtttgttaagtattaactagctagttgccctctttgctatatatatatatatatatatatatatatatatatatatatatatatataaaataggtgagtgtccgtgcgttgcaacagaaatatataataccacgacaagttatat

SEQ ID NO: 10 - DNA, 942 bp, Zea mays
atggtggttcccgtgatcgacttctccaagctggacggcgctgagagggctgaaaccctggcgcagatcgccaatggctgcgaggagtggggattcttccagctcgtgaaccacggcatcccgctggagctgctcgagcgcgtcaagaaggtgtgctccgactgctaccgcctccgggaggccgggttcaaggcgtcggagccggtgcgcacgctggaggcgctcgtcgacgcggagcggcgcggtgaggtggtggcgccggtggacgacctggactgggaggacatcttctacatccacgacggatgccagtggccgtccgacccgccggcgttcaaggagaccatgcgcgagtaccgcgccgagctgaggaagctcgccgagcgagtcatggaggccatggacgagaacctcggcctcgccaggggcaccatcaaggacgccttctccggcggcggccggcacgatcccttcttcggcaccaaggtcagccactacccgccgtgcccacgcccggacctcatcacgggcctgcgcgcgcacaccgacgccggcggcgtcatcctcctgttccaggacgacaaggtcggtggcctggaggtgctcaaggacggcgagtggaccgacgtacagccgctcgagggcgccatcgtcgtcaacaccggcgaccagatcgaggtgctcagcaacgggctgtaccgcagcgcttggcaccgcgtgctgcccatgcgcgacggcaatcgccgctccatcgcatccttctacaacccagccaacgaagccaccatctcgccggcggcggtgcaggccagcggcggtgacgcgtatcccaagtacttgttcggcgattacatggacgtgtacgtcaagcagaagttccaggccaaggagcctaggttcgaagccgtcaagacgggggcgccaaagtcatctccagcggca

SEQ ID NO: 11 - DNA, 1500 bp, Zea mays
atcttcccgagctcgtcttcgatcaattcccaagtcaaataataatataacaacaatggtggttcccgtcatcgacttctccaagctggacggcgctgagagggccgaaaccctggcgcagatcgccaatggctgcgaggagtggggattcttccagctcgtgaaccacggcatcccgctggagcttcttgagcgcgtcaagaaggtgagctccgactgctaccgcctccgggaggccgggttcaaggcgtcggagccggtgcgcacgctggaggcgctcgtcgacgcggagcggcgcggcgaggttgtggcgccggtggatgacctggactgggaggacatcttctacatccacgacggatgccagtggccgtccgagccgccggcgttcaaggagaccatgcgcgagtaccgcgccgagctgaggaagctcgccgagcgcgtcatggaggccatggacgagaacctcggcctcgccaggggcaccatcaaggacgccttctccagcggcggccggcacgagcccttcttcggcaccaaggtcagccactacccgccgtgcccgcgcccggacctcatcacgggcctgcgcgcgcacaccgacgccggcggcgtcatcctgctgttccaggacgacagggtcggcggcctggaggtgctcaaggacggccagtggaccgacgtgcagccgctcgcgggcgccatcgtcgtcaacactggcgaccagattgaggtgctcagcaacgggcgctaccgcagcgcctggcaccgcgtgctgcccatgcgcgacggcaaccgccgctccatcgcttccttctacaacccggccaacgaggccaccatctcgccggcggcggtgcaggccagcggcggcgacgcataccccaagtacgtgttcggcgactacatggacgtgtacgccaagcacaagttccaggccaaggagcccaggttcgaagccgtcaaggttgcagcgcccaagtcatctccagcggcataaataaatggaggggaccaattattaaatgcattataatttatttgttgaataaaacagccggagaaataatgataatgtaaagtatatatgataaacaccggttaggatttaaggtgtttaactttagttgcatggtataatatgatatattgttgtagcaataagtttattaagtattcataagtgttctaaatagtgggctaaggcacttatccatcgcctttctcaaacagaaaatagtgatttaattcgggctatagcgactaatagttgctatatatattaggcgtagtagcaaacaatttcaccctttggaaacagttatatctagaaataactatagccagagatttagaaccttgttaatcatgtagaaattaaaggttcgtcaagtcagagcggcaccgaacaagataaaaatgtgacctcccctatatgcaaatgtctgccaacttattacattggtgggtgccatcttactatgtacaaatatatcgcgg

SEQ ID NO: 12 - DNA, 942 bp, Zea mays
atggtggttcccgtcatcgacttctccaagctggacggcgctgagagggccgaaaccctggcgcagatcgccaatggctgcgaggagtggggattcttccagctcgtgaaccacggcatcccgctggagcttcttgagcgcgtcaagaaggtgagctccgactgctaccgcctccgggaggccgggttcaaggcgtcggagccggtgcgcacgctggaggcgctcgtcgacgcggagcggcgcggcgaggttgtggcgccggtggatgacctggactgggaggacatcttctacatccacgacggatgccagtggccgtccgagccgccggcgttcaaggagaccatgcgcgagtaccgcgccgagctgaggaagctcgccgagcgcgtcatggaggccatggacgagaacctcggcctcgccaggggcaccatcaaggacgccttctccagcggcggccggcacgagcccttcttcggcaccaaggtcagccactacccgccgtgcccgcgcccggacctcatcacgggcctgcgcgcgcacaccgacgccggcggcgtcatcctgctgttccaggacgacagggtcggcggcctggaggtgctcaaggacggccagtggaccgacgtgcagccgctcgcgggcgccatcgtcgtcaacactggcgaccagattgaggtgctcagcaacgggcgctaccgcagcgcctggcaccgcgtgctgcccatgcgcgacggcaaccgccgctccatcgcttccttctacaacccggccaacgaggccaccatctcgccggcggcggtgcaggccagcggcggcgacgcataccccaagtacgtgttcggcgactacatggacgtgtacgccaagcacaagttccaggccaaggagcccaggttcgaagccgtcaaggttgcagcgcccaagtcatctccagcggca

SEQ ID NO: 13 - DNA, 1274 bp, Zea mays
catgcaactaagctttcactgaagcaagcaaacaaacacctaaagatctgctatttgagtatttcttgtttctcttcagcttcatcagccatggtggttcccgtgatcgacttctccaagctggacggcgctgagaggaccgagactctggcgcagatcgccaatggctgcgaggaatggggattcttccagcttgtgaaccatggcatcccgctggagcttcttgagcgcgtcaagaaggtgtgctccgactgctaccgcctccgagaggccgggttcaaggcgtcggagccagtgcgcacgttggaggcgctcgtcgacgcggagcggcgcggcgaggaggtggcgcctgtggatgacctggactgggaggacatattcttcatccacgacggctgccagtggccgtccgacccgtcggcgttcaaggagaccatgcgcgagtaccgcgccgagctgaggaagctcgccgagcgcgtcatggaggccatggacgagaaccttggcctcaccaagggcaccatcaaggatgccttctccgccggcggccggcacgagcccttcttcggcaccaaggtcagccactacccgccgtgcccgcgcccggacctcatcacgggcctgcgcgcgcacaccgacgctggcggagtcatcctgctgttccaggatgacagagtcggtggcctggaggtgctcaaggacggccagtggatcgacgtgcagccgctcgcgggcgccatcgtcatcaacaccggcgatcagatcgaggtgctcagcaacgggcggtaccgcagcgcctggcaccgcgtgctgcccatgcgcgacggcaaccgccgctccatcgcctccttctacaacccggccaacgaggccaccatctcgccggcggcggtgcagggcagcggcggtggtgagacgtaccccaagtacgtgttcggtgattacatggacgtgtatgtcaagcagaagttccaagccaaggagcccagattcgaagccgtcaaggccgcggcgcccaagtcatctccggcggcctaaaacttgcactagacaacttctttatctagtgctaaaacgtttgcggagagttaaaatgtcgggcactctgataaagacaaaatttaccgagtattcgacaaagaactcttctccaatagtgttgccgcttaaggacacaaactcaatacaggatggtaaaattatttgggttgctattttgtttcatcgtgttgagcgtgaaaatgtaatcctaatattcttgttcctcgtgttca

SEQ ID NO: 14 - DNA, 1890 bp, Zea mays
atggtggttcccgtgatcgacttctccaagctggacggcgctgagaggaccgagactctggcgcagatcgccaatggctgcgaggaatggggattcttccagcttgtgaaccatggcatcccgctggagcttcttgagcgcgtcaagaaggtgtgctccgactgctaccgcctccgagaggccgggttcaaggcgtcggagccagtgcgcacgttggaggcgctcgtcgacgcggagcggcgcggcgaggaggtggcgcctgtggatgacctggactgggaggacatattcttcatccacgacggctgccagtggccgtccgacccgtcggcgttcaaggagaccatgcgcgagtaccgcgccgagctgaggaagctcgccgagcgcgtcatggaggccatggacgagaaccttggcctcaccaagggcaccatcaaggatgccttctccgccggcggccggcacgagcccttcttcggcaccaaggtcagccactacccgccgtgcccgcgcccggacctcatcacgggcctgcgcgcgcacaccgacgctggcggagtcatcctgctgttccaggatgacagagtcggtggcctggaggtgctcaaggacggccagtggatcgacgtgcagccgctcgcgggcgccatcgtcatcaacaccggcgatcagatcgaggtgctcagcaacgggcggtaccgcagcgcctggcaccgcgtgctgcccatgcgcgacggcaaccgccgctccatcgcctccttctacaacccggccaacgaggccaccatctcgccggcggcggtgcagggcagcggcggtggtgagacgtaccccaagtacgtgttcggtgattacatggacgtgtatgtcaagcagaagttccaagccaaggagcccagattcgaagccgtcaaggccgcggcgcccaagtcatctccggcggccatggtggttcccgtgatcgacttctccaagctggacggcgctgagaggaccgagactctggcgcagatcgccaatggctgcgaggaatggggattcttccagcttgtgaaccatggcatcccgctggagcttcttgagcgcgtcaagaaggtgtgctccgactgctaccgcctccgagaggccgggttcaaggcgtcggagccagtgcgcacgttggaggcgctcgtcgacgcggagcggcgcggcgaggaggtggcgcctgtggatgacctggactgggaggacatattcttcatccacgacggctgccagtggccgtccgacccgtcggcgttcaaggagaccatgcgcgagtaccgcgccgagctgaggaagctcgccgagcgcgtcatggaggccatggacgagaaccttggcctcaccaagggcaccatcaaggatgccttctccgccggcggccggcacgagcccttcttcggcaccaaggtcagccactacccgccgtgcccgcgcccggacctcatcacgggcctgcgcgcgcacaccgacgctggcggagtcatcctgctgttccaggatgacagagtcggtggcctggaggtgctcaaggacggccagtggatcgacgtgcagccgctcgcgggcgccatcgtcatcaacaccggcgatcagatcgaggtgctcagcaacgggcggtaccgcagcgcctggcaccgcgtgctgcccatgcgcgacggcaaccgccgctccatcgcctccttctacaacccggccaacgaggccaccatctcgccggcggcggtgcagggcagcggcggtggtgagacgtaccccaagtacgtgttcggtgattacatggacgtgtatgtcaagcagaagttccaagccaaggagcccagattcgaagccgtcaaggccgcggcgcccaagtcatctccggcggcc

SEQ ID NO: 15 - DNA, 1133 bp, Zea mays
gctgcatgcaactaagctttcactgaagcaagcaaacaaacacctaaagatctgctatttgagtatttctcgtttctcttcagcttcatcagccatggtggttcccgtgatcgacttctccaagctggacggcgctgagaggaccgagactctggcgcagatcgccaatggctgcgaggaatggggattcttccagcttgtgaaccatggcatcccgctggagcttcttgagcgcgtcaagaaggtatgctccgactgctaccgcctccgggaggccgggttcaaggtgtcggagccagtgcgcacgttggaggcgctcgtcgacgcggagcggcgcggcgaggaggtggcgcctgtggatgacctggactgggaggacatattcttcatccacgacggctgccagtggccgtccgacccgtcggcgttcaagaagaccatacgcgagtaccgcgccgagctgaggaagctcgccgagcgcgtcatggaggccatggacgagaacctcggcctcaccaagggcaccatcaaggatgccttctccggcggcggccggcacgagcccttcttcggcaccaaggtcagccactacccgccgtgcccgcgcccggacctcatcacgggcctgcgtgcgcacaccgacgctggcggagtcatcctgctgttccaggatgacagagtcggtggcctggaggtgctcaaggacggccagtggatcgacgtgcagccgctcgcgggcgccatcgtcatcaacaccggcgatcagatcgaggtgctcagcaacgggcggtaccgcagcgcctggcaccgcgtgctgcccatgcgcgacggcaaccgccgctccattgcctccttctacaacccggctaacgaggccaccatctcgccggcggcggtgcagggcagcagcggtggtgagacgtaccccaagtacgtgttcggtgattacatggacgtgtatgtcaagcagaagttccaagccaaggagcccagattcgaagccgtcaaggccgcggcgcccaagtcatctccggcggcctaaaacttgcactagacaacttctttatctagtgctaaaacgtttgcggagagttaaatgttgggcactcgataaagacaaagtttaacgagta

SEQ ID NO: 16 - DNA, 945 bp, Zea mays
atggtggttcccgtgatcgacttctccaagctggacggcgctgagaggaccgagactctggcgcagatcgccaatggctgcgaggaatggggattcttccagcttgtgaaccatggcatcccgctggagcttcttgagcgcgtcaagaaggtatgctccgactgctaccgcctccgggaggccgggttcaaggtgtcggagccagtgcgcacgttggaggcgctcgtcgacgcggagcggcgcggcgaggaggtggcgcctgtggatgacctggactgggaggacatattcttcatccacgacggctgccagtggccgtccgacccgtcggcgttcaagaagaccatacgcgagtaccgcgccgagctgaggaagctcgccgagcgcgtcatggaggccatggacgagaacctcggcctcaccaagggcaccatcaaggatgccttctccggcggcggccggcacgagcccttcttcggcaccaaggtcagccactacccgccgtgcccgcgcccggacctcatcacgggcctgcgtgcgcacaccgacgctggcggagtcatcctgctgttccaggatgacagagtcggtggcctggaggtgctcaaggacggccagtggatcgacgtgcagccgctcgcgggcgccatcgtcatcaacaccggcgatcagatcgaggtgctcagcaacgggcggtaccgcagcgcctggcaccgcgtgctgcccatgcgcgacggcaaccgccgctccattgcctccttctacaacccggctaacgaggccaccatctcgccggcggcggtgcagggcagcagcggtggtgagacgtaccccaagtacgtgttcggtgattacatggacgtgtatgtcaagcagaagttccaagccaaggagcccagattcgaagccgtcaaggccgcggcgcccaagtcatctccggcggcc

SEQ ID NO: 17 - DNA, 1220 bp, Zea mays
gaacaacacaaattaagtagtggagtgtcagaacttgggaggcacaaattaagtacaaagcagtctaattaatgacgggcccgatggagattccggtgatcgatctcggcggcctcaacggcggcggcgaggagaggtcgcggaccttggcggagctccacgacgcctgcaaggactggggcttcttctgggtggagaaccacggcgtggacgcgccgctgatggacgaggtcaagcgcttcgtctacggccactacgaggagcacctggaggccaagttctacgcctccgccctcgccatggacctcgaggccgccaccagaggtgacactgatgagaagccctccgacgaggtggactgggagtccacctacttcatccagcaccaccccaagaccaacgtcgccgacttcccagagatcacgccgccgacacgagagacgctggacgcgtacgtcgcgcagatggtgtccctcgcggagcgtctggccgagtgcatgagcctcaacctgggcctccccggggcccacgtcgccgccaccttcgcgccgccgttcgtgggcaccaagttcgccatgtacccgtcctgcccgcgcccggagctggtgtggggcctgcgcgcgcacaccgacgccggcggcatcatcctgctcctccaggacgacgtcgtgggcggcctcgagttcctcagggccggcgcccactgggtccccgtcggccccaccaaggggggcaggctcttcgtcaacatcggggaccagatcgaggtcctcagcgccggcgcctaccggagcgtcctgcaccgcgtcgcggccggggaccagggccgccgcctgtccgtggccacgttctacaaccctggcaccgacgccgtggtcgcgccggcgccccgcagggatcaggacgccggcgccgcggcgtaccccggtccctacaggttcggggactacctcgactactaccagggcaccaagttcggcgacaaggacgccaggttccaggccgtcaagaagctgctcggctaagcgaacagctgcaagtaggcagaggcagcttagctcgtggactatgcatagtttcaagcttgctgcttgcttcttgttcgatccattgtctgcatgcgtactgttgcgtgtttaaatttagcaaatcttatacgtagtcgttactggtactacgtattctgtggttgacaatacattgttgcggtttaagggcgc

SEQ ID NO: 18 - DNA, 951 bp, Zea mays
atgacgggcccgatggagattccggtgatcgatctcggcggcctcaacggcggcggcgaggagaggtcgcggaccttggcggagctccacgacgcctgcaaggactggggcttcttctgggtggagaaccacggcgtggacgcgccgctgatggacgaggtcaagcgcttcgtctacggccactacgaggagcacctggaggccaagttctacgcctccgccctcgccatggacctcgaggccgccaccagaggtgacactgatgagaagccctccgacgaggtggactgggagtccacctacttcatccagcaccaccccaagaccaacgtcgccgacttcccagagatcacgccgccgacacgagagacgctggacgcgtacgtcgcgcagatggtgtccctcgcggagcgtctggccgagtgcatgagcctcaacctgggcctccccggggcccacgtcgccgccaccttcgcgccgccgttcgtgggcaccaagttcgccatgtacccgtcctgcccgcgcccggagctggtgtggggcctgcgcgcgcacaccgacgccggcggcatcatcctgctcctccaggacgacgtcgtgggcggcctcgagttcctcagggccggcgcccactgggtccccgtcggccccaccaaggggggcaggctcttcgtcaacatcggggaccagatcgaggtcctcagcgccggcgcctaccggagcgtcctgcaccgcgtcgcggccggggaccagggccgccgcctgtccgtggccacgttctacaaccctggcaccgacgccgtggtcgcgccggcgccccgcagggatcaggacgccggcgccgcggcgtaccccggtccctacaggttcggggactacctcgactactaccagggcaccaagttcggcgacaaggacgccaggttccaggccgtcaagaagctgctcggc

SEQ ID NO: 19 - DNA, 1451 bp, Zea mays
atccaatatccactgcaccacttctgctaatcccttgttcttgtgcctccgatccggagctctcaccattgtcatcgtcaatcgatcaatataaagcgagccaattaccccaaggagctaccgcttgcgacggtatggcgatcccggtgattgacttctccaagctggacggccctgagagggccgagaccatggcggccctcgctgccgggttcgagcacgtggggttcttccagctggtgaacaccggcatctccgacgacctgctggagcgggtgaagaaggtgtgcagcgactcctacaagctgcgggacgaggcgttcaaggactccaaccccgcggtgaaggcgctcacagagctcgtggacaaggagatcgaggacggcctccccgcgaggaagataaaggacatggactgggaggacgtcttcaccctccatgacgacctgccatggccttccaaccctcccgccttcaaggagacgatgatggagtaccgcagggagctgaagaagctggcggagaagatgctgggcgtgatggaggagctgctggggttggaggagggccacatcaggaaggccttcagcaacgacggcgagttcgagcccttctacggcaccaaggtcagccactacccgccgtgcccgcggccggacctcatcgacggcctgcgcgcgcacaccgacgccggcggcctcatccttctgttccaggatgaccgcttcggcggcctgcaggcgcagcttccggacggcagctgggtcgacgtccagcccctcgagaacgccatcgtcatcaacaccggcgaccagatcgaggtgctgagcaatggccggtacaagagcgcatggcaccgcatcctggcgacccgcgacggcaaccggcgctccatcgcctccttctacaacccagcgcgcctggccaccatcgctccggcgatccccgccgcaggggtcggcgacgacgactacccgagcttcgtgttcggcaactacatggaggtgtacgtcaagcagaagttccagcctaaggcgcccagatttgaagccatggccacgacgacgaccaagtgatgacctagcagcgactcagcgagagcctaaataaatattaattcacagtcgtcaagttaatcttgtggttatacggtacgggcggggcttgtacttatgtaggttgctaagtcttaagtgtgtagtttaattaacgtgtgtgtggaatgtacgcgtcatacaaatgtgttggtgtgtgccctgccgcaagattgcggtgagcggtggatctatggtcaacgggtgcctaaatgatttgtgcttttgtagcataaaatggcacatctcctctgcttttgttacatctccaccttttctttttgcacttttcacctcaagtaaaacatgtggcggctttcactaagtacaaagaagctctacagagctatttctatt

SEQ ID NO: 20 - DNA, 939 bp, Zea mays
atggcgatcccggtgattgacttctccaagctggacggccctgagagggccgagaccatggcggccctcgctgccgggttcgagcacgtggggttcttccagctggtgaacaccggcatctccgacgacctgctggagcgggtgaagaaggtgtgcagcgactcctacaagctgcgggacgaggcgttcaaggactccaaccccgcggtgaaggcgctcacagagctcgtggacaaggagatcgaggacggcctccccgcgaggaagataaaggacatggactgggaggacgtcttcaccctccatgacgacctgccatggccttccaaccctcccgccttcaaggagacgatgatggagtaccgcagggagctgaagaagctggcggagaagatgctgggcgtgatggaggagctgctggggttggaggagggccacatcaggaaggccttcagcaacgacggcgagttcgagcccttctacggcaccaaggtcagccactacccgccgtgcccgcggccggacctcatcgacggcctgcgcgcgcacaccgacgccggcggcctcatccttctgttccaggatgaccgcttcggcggcctgcaggcgcagcttccggacggcagctgggtcgacgtccagcccctcgagaacgccatcgtcatcaacaccggcgaccagatcgaggtgctgagcaatggccggtacaagagcgcatggcaccgcatcctggcgacccgcgacggcaaccggcgctccatcgcctccttctacaacccagcgcgcctggccaccatcgctccggcgatccccgccgcaggggtcggcgacgacgactacccgagcttcgtgttcggcaactacatggaggtgtacgtcaagcagaagttccagcctaaggcgcccagatttgaagccatggccacgacgacgaccaag

SEQ ID NO: 21 - protein, 326 aa, Zea mays
MAPALSFPIIDMGLLAGEERPAAMELLQDACENWGFFEILNHGISTELMDEVEKLTKEHYKRVREQRFLEFASKTLGDGRDIAQGVKAENLDWESTFFVRHLPEPNIAEIPDLDDEYRRVMKRFAGELEALAERLLDLLCENLGLDRGYLARAFRGPSKGAPTFGTKVSSYPPCPRPDLVSGLRAHTDAGGIILLFQDDRVGGLQLLKDGEWVDVPPMRHAVVVNLGDQLEVITNGRYKSVMHRVVAQPSGNRMSIASFYNPGSDAVIFPAPALVKAEEAAAGAYPSFVFEDYMKLYVRHKFEAKEPRFEAFKSMETDSSNRIAIA

SEQ ID NO: 22 - protein, 317 aa, Zea mays
MAATVSSFPVVNMEKLETEERATAMEVIRDGCENWGFFELLNHGISHELMDEVERLTKAHYATFREAKFQEFAARTLEAGEKGADVKDVDWESTFFVRHLPASNLADLPDVDDRYRQVMEQFASEIRKLSERLLDLLCENLGLEPGYLKAAFAGSDGPTFGTKVSAYPPCPRPDLVDGLRAHTDAGGIVLLFQDDQVSGLQLLRGGEWVDVPPMRHAIVANVGDQLEVITNGRYKSVMHRVLTRPDGNRMSVASFYNPGADAVIFPAPALVGAAEEDRAEAAYPSFVFEDYMNLYVRHKFEAKEPRFEAMKSAIATA

SEQ ID NO: 23 - protein, 317 aa, Zea mays; identical to SEQ ID NO: 22

SEQ ID NO: 24 - protein, 323 aa, Zea mays
MAATVSFPVVNMEKLETEERDTAMAVIRDACENWGFFELLNHGISHELMDEVERLTKAHYATFREAKFQEFAARTLAAAGDEGADVSDVDWESTFFVRHLPASNLADLPDVDDHYRQVMKQFASEVQKLSEKVLDLLCENLGLEPGYLKAAFAGSDGGPTFGTKVSAYPPCPRPDLVAGLRAHTDAGGLILLLQDDQVSGLQLLRGGDGGEWVDVPPLRHAIVANVGDQLEVVTNGRYKSAVHRVLARPDGNRMSVASFYNPGADAVIFPAPALVGEEERAEKKATTYPRFVFEDYMNLYARHKFEAKEPRFEAMKSSAIATA

SEQ ID NO: 25 - protein, 314 aa, Zea mays
MVVPVIDFSKLDGAERAETLAQIANGCEEWGFFQLVNHGIPLELLERVKKVCSDCYRLREAGFKASEPVRTLEALVDAERRGEVVAPVDDLDWEDIFYIHDGCQWPSDPPAFKETMREYRAELRKLAERVMEAMDENLGLARGTIKDAFSGGGRHDPFFGTKVSHYPPCPRPDLITGLRAHTDAGGVILLFQDDKVGGLEVLKDGEWTDVQPLEGAIVVNTGDQIEVLSNGLYRSAWHRVLPMRDGNRRSIASFYNPANEATISPAAVQASGGDAYPKYLFGDYMDVYVKQKFQAKEPRFEAVKTGAPKSSPAA

SEQ ID NO: 26 - protein, 314 aa, Zea mays
MVVPVIDFSKLDGAERAETLAQIANGCEEWGFFQLVNHGIPLELLERVKKVSSDCYRLREAGFKASEPVRTLEALVDAERRGEVVAPVDDLDWEDIFYIHDGCQWPSEPPAFKETMREYRAELRKLAERVMEAMDENLGLARGTIKDAFSSGGRHEPFFGTKVSHYPPCPRPDLITGLRAHTDAGGVILLFQDDRVGGLEVLKDGQWTDVQPLAGAIVVNTGDQIEVLSNGRYRSAWHRVLPMRDGNRRSIASFYNPANEATISPAAVQASGGDAYPKYVFGDYMDVYAKHKFQAKEPRFEAVKVAAPKSSPAA

SEQ ID NO: 27 - protein, 315 aa, Zea mays
MVVPVIDFSKLDGAERTETLAQIANGCEEWGFFQLVNHGIPLELLERVKKVCSDCYRLREAGFKASEPVRTLEALVDAERRGEEVAPVDDLDWEDIFFIHDGCQWPSDPSAFKETMREYRAELRKLAERVMEAMDENLGLTKGTIKDAFSAGGRHEPFFGTKVSHYPPCPRPDLITGLRAHTDAGGVILLFQDDRVGGLEVLKDGQWIDVQPLAGAIVINTGDQIEVLSNGRYRSAWHRVLPMRDGNRRSIASFYNPANEATISPAAVQGSGGGETYPKYVFGDYMDVYVKQKFQAKEPRFEAVKAAAPKSSPAA

SEQ ID NO: 28 - protein, 315 aa, Zea mays
MVVPVIDFSKLDGAERTETLAQIANGCEEWGFFQLVNHGIPLELLERVKKVCSDCYRLREAGFKVSEPVRTLEALVDAERRGEEVAPVDDLDWEDIFFIHDGCQWPSDPSAFKKTIREYRAELRKLAERVMEAMDENLGLTKGTIKDAFSGGGRHEPFFGTKVSHYPPCPRPDLITGLRAHTDAGGVILLFQDDRVGGLEVLKDGQWIDVQPLAGAIVINTGDQIEVLSNGRYRSAWHRVLPMRDGNRRSIASFYNPANEATISPAAVQGSSGGETYPKYVFGDYMDVYVKQKFQAKEPRFEAVKAAAPKSSPAA

SEQ ID NO: 29 - protein, 317 aa, Zea mays
MTGPMEIPVIDLGGLNGGGEERSRTLAELHDACKDWGFFWVENHGVDAPLMDEVKRFVYGHYEEHLEAKFYASALAMDLEAATRGDTDEKPSDEVDWESTYFIQHHPKTNVADFPEITPPTRETLDAYVAQMVSLAERLAECMSLNLGLPGAHVAATFAPPFVGTKFAMYPSCPRPELVWGLRAHTDAGGIILLLQDDVVGGLEFLRAGAHWVPVGPTKGGRLFVNIGDQIEVLSAGAYRSVLHRVAAGDQGRRLSVATFYNPGTDAVVAPAPRRDQDAGAAAYPGPYRFGDYLDYYQGTKFGDKDARFQAVKKLLG

SEQ ID NO: 30 - protein, 313 aa, Zea mays
MAIPVIDFSKLDGPERAETMAALAAGFEHVGFFQLVNTGISDDLLERVKKVCSDSYKLRDEAFKDSNPAVKALTELVDKEIEDGLPARKIKDMDWEDVFTLHDDLPWPSNPPAFKETMMEYRRELKKLAEKMLGVMEELLGLEEGHIRKAFSNDGEFEPFYGTKVSHYPPCPRPDLIDGLRAHTDAGGLILLFQDDRFGGLQAQLPDGSWVDVQPLENAIVINTGDQIEVLSNGRYKSAWHRILATRDGNRRSIASFYNPARLATIAPAIPAAGVGDDDYPSFVFGNYMEVYVKQKFQPKAPRFEAMATTTTK

SEQ ID NO: 31 - DNA, 2256 bp, Zea mays
cagcccagccaagccaagctggagtgcaagagaatcccgtgcgtgcatgctgagggcccgcgacgagacgggccaacacgcgtcgcccacatgggcgtggcccgcgtgggtgcccacaggtcaatgccctgtctgtcagcaagagcaacaaccaaaaaacaactctgctgctggctgctgtctgttgacaagtcgggaaagctcgtccacttccagttccactccgctagaaagcttgaacttggatgccgagcctataaatggcgaccgaccccggccacttccactcaccgcactccagcgttcagcattagacacgagagctcctagtagccagaccagtagtcccgcgaccctgtcgagagaaacagacagagcaacatggcgcctgcattgtcattcccgatcatcgacatggggctgctcgccggggaggagaggccggcggcgatggagctgctgcaagatgcgtgcgagaactggggcttcttcgaggtagatgctcagcatggatggagaactgatccaactccaggaactgaaacaaaataattaagctgcacaaatatacactctatctgtttttatcgttgatgatggtgctctatctgttttctttaatcctattattccctcctgccctgcagattctgaaccacggcatctcgacggagctgatggacgaggtagagaagctgaccaaggagcactacaagcgggtgcgcgagcagaggttcctcgagttcgccagcaagacgctcggggacggccgcgacattgcgcagggcgtgaaggcggagaacctggactgggagagcaccttcttcgtccgccacctcccggagcccaacatcgccgagataccggacctggacgacgagtaccggcgcgtcatgaagcggttcgccggcgagctggaggcgctggcggagcggctgctggacctgctgtgcgagaacctcggcctcgacaggggctacctggcgcgcgcgttccgcgggcccagcaagggcgccccgacgttcggcaccaaggtcagcagctacccgccgtgcccgcgcccggacctcgtcagcggcctgcgcgcgcacaccgacgccggcggcatcatcctgctgttccaggacgaccgggtgggcggcctccagctgctcaaggacggcgagtgggttgacgtgccgcccatgcgccacgccgtcgtcgtcaacctgggcgaccagctggaggtgatcaccaacggcaggtacaagagcgtcatgcaccgggtggtggcgcagcccagcgggaacaggatgtccatcgcgtccttctacaacccgggcagcgacgcggtcatcttcccggcgccggcgctggtcaaggccgaggaggcggcagcgggggcgtaccccagcttcgtcttcgaggactacatgaagctgtacgtgcggcacaagttcgaggccaaggagccacggttcgaggccttcaagtccatggagacggacagctccaatcgcatagccatcgcgtgaaacaccggacctgcgccgagctctggcttactgttcgagatgtacgtgcggcgtactgtactcactaccggaatccgagactttgccgagtgttggcttctttgccgagtgccttttgtcgggcactcggcaaagaaagctttgccgagtgccgtactcggtaacgttaggcactcggcaaaacgtgctttgccgagggctgaacactcggcacagaacggcactcggcaaagacaactttgccgagagtcaaacactcggcaaaggaggctctcggcgagcggccgtcccaaagctgacggccgttagtctttgccgagtgtcatccgttggctctcggcaaagaggttctgtaccgagtgccacatagtaggcactcggcaaagcatactttgccgagtgtcatctctggacactcggcaaagtatatttttatttttttattttgtctctcgaaatttttgtggtatgtttctacactatgtagacctacatgtaccattttgggataattataacagttttttctatagctagtatatttagtttgtttatttgaatttcttcggaaaattcagatttgaactgcaggtcactcgaaacttggaaaaccgtgaatgcaaaaatgatatccatgctacatagcacaagttacgaccgatttcaggagcgaaccggaaacttcgagcaccatgctcactcaacatgaccgtaaactgccatgacgaatctctagatcgta

SEQ ID NO: 32 - DNA, 2923 bp, Zea mays
tacggtactgcacatccggactgtcctgtcccagcctcccaggttgcatgctcatctacaccgtcgagcgtcgaggcggctagctctagccgatcagcgagcatcgcgggcggcgggctatatacgtccagactgcttttatttgagaatgcgtagtttggcttcctaatccatctgactaaactatgaaagtaataataaacgtaccgtcgcgaggccattctggtaatccaacatttctcgctcagccgcctataaattgggccgcgcgcaccgcctcgctctccactcaaacaaactcaagcctgccctgtcctgccttgttaagcaacacagcgagacatcacgagagctagagagagatggcggccacggtttcctccttcccggtggtgaacatggagaagctggagacagaggagagggccacggccatggaggtcatccgcgacggctgcgagaactggggcttcttcgaggtgtgcatatacatacatactctgcagactgcttgctgctcacacgaagctaccacagaacacaattattctactaacctacgcaccacacctgatcacaataagtaatgatctaaccacacacagcaggaagaattactacttcacttgttgtttgcctgacctgccacccccctgcttcttcaacatctagagccccttcattctgtcagcacatgcaggctgttcgtttcggattaaatcaatctagttgttcctgacagtagaaaccgatacacattaaagcgagcaccgttccagaaaaagaaaagcaaaacaaagtattctagcagcttgctttacctaacaaacagccaacgatcctcgaacgtacagattcctattctccatgccatcaaccggccgaccaccagctgattccatcacgtctttctctcaccgcgcctagctgatcagcacacacacaagtagcatcttatctattggttcgttgatgcccagctctcgaacgaatcaccatctcatgtattgtcttgtccccatccccatgcatgcagctgctgaaccacggcatctcgcacgagctgatggacgaggtggagcggctgaccaaggcgcactacgccaccttccgggaggccaagttccaggagttcgcggcgcggacgctggaggccggcgagaagggcgccgacgtcaaggacgtggactgggagagcaccttcttcgtccgccacctcccggcctccaacctcgccgacctccccgacgtcgacgaccgctacaggtgcgttcagacctcaaacacaacactacgtgcgtgcgtgcggatgcgcgccattaaattaatgacgtgtggatcagtatcatatattattagtgtttatctgctagctgcgacccaatgatcagtcgtctttgttaatcgactttttgttggcttctctcggaatgttctaagtgccatgtcacccgcttttgactgatcagtttattttaattgatctgattagtcttagcttgagagtgacttgagtataccaggctgggatactacctgacctgacctgctcctacataacggattaagtaatgtttcgagaaattttgtccatacgcatataattaagttatcagaattctgcctgacgacgacgacgacgcgaaaacagttagttatctgttcatctcgttgcctttaattgcttgacaagctagctagctgtacagcagaatgcggtgcgagccccgtagctatgacaaggtcgaatcgcctttcagcaggcgacagcgctgtttgtccggtggaatattccggccgtgtctcaaagccttccttccttccgtgtcgctgcaggcaggtgatggagcagttcgcatcggagatccggaagctgtcggagaggctgctggacctgctgtgcgagaacctgggcctggagcccgggtacctgaaggcggccttcgcggggtcggacggcccgacgttcggcaccaaggtgagcgcgtacccgccgtgcccgcgcccggacctcgtcgacggcctccgcgcgcacaccgacgccggcggcatcgtgctgctgttccaggacgaccaggtgagcggcctgcagctgctcaggggcggggagtgggtggacgtgccgcccatgcgccacgccatcgtcgccaacgtcggcgaccagctggaggtcatcaccaacgggcggtacaagagcgtcatgcaccgcgtgctcacgcgccccgacggcaaccgcatgtccgtcgcgtccttctacaacccgggcgccgacgccgtcatcttcccggcgcccgcgctcgtcggcgccgccgaggaggaccgcgccgaggccgcgtacccgagcttcgtgttcgaggactacatgaacctgtacgtgcgccacaagttcgaggccaaggagcccaggttcgaggccatgaagtcggccatcgccaccgcgtgagaaagactgccttccgctgccggcttccttcgtggcgtcaagccttgaggcttgaacgaacaacgtacgtccatgtgcttatagtggcacagttgtgtgtgtaactaccgatcgtggaacggcctaatgtatttcggttgcctcagatcgatctatatgtgcgtatacattatgtactcaaaagtgtgtagcgtctggttaatgtacgagcagtgtgtatgtgaccaggacccggtgtgtagttgctattactaccatatccggtgaatgatcaaaccttttggtgtattaaaactagatgttcatccccctcacggactcaccccaggtattgacaaccaaatcggaatatggcatatataataaaaacatgatgtcccggccaagaaaggggactattcgaaaaaccaaaaattgcgtaaagggacccttggacaagtcaaaccatagtatttagtgtacatgtgctagaaatttgta

SEQ ID NO: 33 - DNA, 3306 bp, Zea mays
attatttcgaatacggtactgcacatccggactgtcctgtcccagcctctcccaggttgcatgctcatctacaccgtcgagcgtcgaggcggctagctctagccgatcagcgagcatcgcgggctatatacgtccagactgctttcatttgagaatgcgtagtttggcttcctaatccatttgagtaaattatgaaagtaatgataaacgtaccgtcgcgaggtcactctggtaatccaacatttctcgctcagccgcctataaattgggccgcgcgcaccgcctcgctctccactcaaacaaactcaagcctgccctgccctgccttgttaagcaaagcaacccagctgcgagacacgagagctagctagagagagatggcggccacggtttcctccttcccggtggtgaacatggagaagctggagacagaggagagggccacggccatggaggtcatccgcgacggctgcgagaactggggcttcttcgaggtgtgcatatacatactctgcagactgcttgctgctcacaccaagctaccacagaacacaattattctactaaccaacgcaccacacctgatcacaataagtaatgatctaaccacacagcaggaagaattactacttcacttgttgtttgcctgacctgccacccccctgcttcttcaacatctagagccccttcattctgtcagcacatgcaagctgttcgtttcggatcaaatctatttgttcggactgctgacagtagaaaccgatactcgttaaagccagcaccaccgttccagaaaaagaaaagcaaaacaaagtattctagcagcttgctttacctaacaaacagcctccgatcctcgaacgtacagattcctattctccatgccatcaaccggccgaccaccagctgattccatcacgtctctctctcaccgcgcctagctgatgagcacacacaaagtagcatcttatctattggttcgttgatgcccagctctcgaacgaatcaccatctcatgtattgtcttgtccccatccccatgcatgcagctgctgaaccacggcatctcgcacgagctgatggacgaggtggagcggctgaccaaggcgcactacgccaccttccgggaggccaagttccaggagttcgcggcccggacgctggaggccggcgagaagggcgccgacgtcaaggacgtggactgggagagcaccttcttcgtccgccacctcccggcctccaacctcgccgacctccccgacgtcgacgaccgctacaggtgcgttcagacctcaaacacaacactacgtgcgtgcgtgcgtgctagctagctagcttatgcgcgccattaaattaatgacgtctggcgcacagggccgggccggcataattgaaggccctgtactgtttttttttcttttttttctttgttaagaatagatgatacagattaatctcatttattaacagtgattgaattattaatgtaggaaatggcttaataacgataacaaatgatcttaaagtttggattttatgctagcatgtgctagctgcacttcgccatatagccaaaataagttgcatgagagattggtactcgcttgttacgacaaacactatgttttattcttatcgagctgacttagctagactttctaatcattactaaaatttatattgattaaattatcactaactattattttaggggcccttgaagggagggggccctgttcttgtgcactagtgacacatgcctcccgcccgggcctgctggcgcagtatcgtatatttattagtgtttggctgctagctgcgacccaatgatcagtcgtctttgttaatcgactttttgttggcttctgacggatgttctaagtgccatgtcacccgcttttgactgatcagtttattttaattgatctgattagtcttagcttgagagtgacttgagtatagcaggctgggatactacctgacctgctcctacataacggattaagtaatgtttcaagaaattttgtccatacgcatataattaagttatcattatcagaattctgcctgacgacgacgacgacgacgcgaaaacagttagttatctgttcatctcgttgcctttaattgcttgacaagctagctagctagctgtacagcagaatgcggtgcgagccccgtagctatgacaaggtcgatcgaatcgccttttcagcaggcgacagcgctatttgtccggtggaattattccggccgtgtctcaaagccttccttccgtacgtgtcgctgcaggcaggtgatggagcagttcgcatcggagatccgcaagctgtcggagaggctgctggacctgctgtgcgagaacctgggcctggagcccgggtacctgaaggcggccttcgcggggtcggacggcccgacgttcggcaccaaggtgagcgcgtacccgccgtgcccgcgcccggacctcgtcgacggcctccgcgcgcacaccgacgccggcggcatcgtgctgctgttccaggacgaccaggtgagcggcctgcagctgctcaggggcggggagtgggtggacgtgccgcccatgcgccacgccatcgtcgccaacgtcggcgaccagctggaggtgatcaccaacgggcggtacaagagcgtcatgcaccgcgtgctcacgcgccccgacggcaaccgcatgtccgtcgcgtccttctacaacccgggcgccgacgccgtcatcttcccggcccccgcgctcgtcggcgccgccgaggaggaccgcgccgaggccgcgtacccgagcttcgtgttcgaggactacatgaacctgtacgtgcgccacaagttcgaggccaaggagcccaggttcgaggccatgaagtcggccatcgccaccgcgtgagagaagactgccttccgctgcaggcttccttcgtggcgtcaagccttgaggcttgaacgaacaacgtacgtccatgtgcttatagtggcacagttatgtgtgtaactaccgatcgtggaacggcctaatgtatttcggttgcctcagatcgatctatatgtgcgtatacattatgtactgaaaagtgtgtagcgtctggttaatgtatgagcagtgtgtatgtgaccgggacccggtgtgtagttgctattactaccatatccggtgaatgatcaaaccttttggtgtattaaaactagatgttcatcccctcacggactaccctggtattgacaaccaaaacggaatatgacatatatagtaaaaacatgatttcccggccaagaaaggggactattccaactcgg

SEQ ID NO: 34 - DNA, 2844 bp, Zea mays
caaagtatgggattgttaggctcgataaaaaaacctagtggtcgtcatagaaaaatagcatgcaccacagccgtgacatcgttggatatttatattactaccttatatccagcgcttacttttctgggatttaaacacactcaatctaaatagatttagaaaaaaacgaaccgcttcgtctcccaggtagtcagtcttgcatagttgggcctcgcgcgaggttattctggtaatctcgcatcctggcgctcggcctataaactgggccgcacccgccgcctcaatctccacacaaagcttggcctgcctgttaagcaacccggcgagcgaggtggtgagagaacgagcgagagggagatggcagccacggtgtccttcccggtggtgaacatggagaagctggagaccgaggagagggacacggccatggcggtcatccgcgacgcctgcgagaactggggcttcttcgaggtgtgtgcatatatctcatagagactcacatcaagcacgcacggaacaactaaggccctgtttggaattgtagtatttttgcagctttgaaacaatactatggtatttaatgatactatagtattagagctcaaaaggtgtttggtttgtacagtcaaaacacagttttaaataccatggtttacccaaaactgtggtatttttggagtttttgaaactccactcaggacctcagttttcttctcttctctctacatatactttgtttttccaatagaaccaaacagacctcggttttgaacaataccaacgcaacgcaccacacaccggccgtatgtatctcccattaagtaatgtaaccacacgcacaacagtcgttttctgcatcaactagtatctcgttggctcaaagcctcgatcagcagagcatgtaattctggtgatcttttcgccccatgcatgcagctgctgaaccatggcatctcgcacgagctgatggacgaggtggagcggctgaccaaggcgcactacgccaccttccgggaggccaagttccaggagttcgcggcgcggacgctggccgcggccggcgacgagggcgccgacgtcagcgacgtggactgggagagcaccttcttcgtccgccacctcccggcctccaacctcgccgacctccccgacgtcgacgaccactaccggtacgttgcgtccaaacacgctaccgtgctagctagctagctagctgcgtgtcgttaacgacgacgtgcgtgtagtatcgtattcttagtgggtgttaaactttttgttggcttctttctgacataaaattccaagtggtgccatgtcaccggcttttgactcttgtctctaaaccattttaaaaagaaaaatctgaatataatctcaactggagcgatcaacaaacgtacaaaatactaccagacctgacctgctcctatcaacgaatgaagcagtgcagtgggggtagtagcgtgcagtgtgtttccattccatccacagcatcagaattcttgcctgacgtcgacgacgcgcatagttatccgatcattccgttgcccctgtcaagtgtacatcagattgaatgctgtgttaggccagcaactatcacaatcacaagtcatagcaggtgacggtgcgatcgacgcgctttgtttggtggaacattttcccgtgttcaattctttcttcctttctttttttttttaaaaaaaaaggctttccgtgtcgctgctgcaggcaagtgatgaagcagttcgcatcggaggtgcagaagctgtcggagaaggtgctggacctgctgtgcgagaacctgggcctggagcccgggtacctgaaggcggccttcgcggggtcggacggcggcccgacgttcggcaccaaggtgagcgcgtacccgccgtgcccgcgcccggacctggtggccggcctgcgcgcgcacaccgacgccggcggcctcatcctgctgctccaggacgaccaggtgagcgggctgcagctgctcaggggcggcgacggcggggagtgggtggacgtgccgccgctgcgccacgccatcgtcgccaacgtcggcgaccagctggaggtggtcaccaacgggcggtacaagagcgcggtgcaccgcgtgctcgcccgccccgacggcaaccgcatgtccgtcgcgtccttctacaacccgggcgccgacgccgtcatcttcccggcccccgcgctcgtcggcgaggaggagcgagccgagaagaaggccaccacgtacccgaggttcgtgttcgaggactacatgaacctgtacgcgcgccacaagttcgaggccaaggagccccggttcgaggccatgaagtcgtcggccatcgccaccgcgtgagcacataatactgccgtgttctcccttcgtggggtgcatatgcttgagcttgaagagccatgtgcctgtatgtagtggcacgtacggtggttatgcgtgtatcgtggaatggcgcggcgtgatgtattttggttgtctcagatctaagtgtgtgcgtatatattgtgtactgtaaagtttgcagcgtctgattaatgtacgagcagtgtgtgtacctaaccagaacctggaatgtggctggctgtgtgctgatattactaccacatcaggtgagtggccacccgtcgtcgcctcctacggctccggtgccgactcgaccccttccttccctgcgaccctgcggccccaccgcccttatctccatggatacttgcggcgagcaaaggcttaacaaaggagaacagtgtgcaaaacatacctgcagtgagcaaaggctttacatgaggatatcaggatatgcacagacctaccatacaagctatagcctttcc

SEQ ID NO: 35 - DNA, 1738 bp, Zea mays
ttggctggcaatgtggtcaccttgacagtgacatccgatcgatcctgtggcgtatcctgaatttgccaccacaagcatccaatccaattgctctcccactgcccagaagcttcatcacacctcagctagaggcagccatgcatggcaggaccaaaaagcggtccagtccaggtccgtacctgagagacttgtgttgaccctcctcatccatggcagtaggtaggttgagctgctcgttgatcactgctattatatatacgggtgccatggattcatgccttctccatcctcaagtcatcagctagctagccttccctacagcaactgcatacatacaacacttccatctgcccgctcgtcttcgatcaattcccaagtcaaataataatataacagcaatggtggttcccgtgatcgacttctccaagctggacggcgctgagagggctgaaaccctggcgcagatcgccaatggctgcgaggagtggggattcttccagctcgtgaaccacggcatcccgctggagctgctcgagcgcgtcaagaaggtgtgctccgactgctaccgcctccgggaggccgggttcaaggcgtcggagccggtgcgcacgctggaggcgctcgtcgacgcggagcggcgcggtgaggtggtggcgccggtggacgacctggactgggaggacatcttctacatccacgacggatgccagtggccgtccgacccgccggcgttcaaggagaccatgcgcgagtaccgcgccgagctgaggaagctcgccgagcgagtcatggaggccatggacgagaacctcggcctcgccaggggcaccatcaaggacgccttctccggcggcggccggcacgatcccttcttcggcaccaaggtcagccactacccgccgtgcccacgcccggacctcatcacgggcctgcgcgcgcacaccgacgccggcggcgtcatcctcctgttccaggacgacaaggtcggtggcctggaggtgctcaaggacggcgagtggaccgacgtacagccgctcgagggcgccatcgtcgtcaacaccggcgaccagatcgaggtgctcagcaacgggctgtaccgcagcgcttggcaccgcgtgctgcccatgcgcgacggcaatcgccgctccatcgcatccttctacaacccagccaacgaagccaccatctcgccggcggcggtgcaggccagcggcggtgacgcgtatcccaagtacttgttcggcgattacatggacgtgtacgtcaagcagaagttccaggccaaggagcctaggttcgaagccgtcaagacgggggcgccaaagtcatctccagcggcataaataaacagggaaaacaattattgaatgcattattaaaaggtagtaataagtttgttaagtattaactagctagttgccctctttgctatatatatatatatatatatatatatatatatatatataaaataggtgagtgtccgtgcgttgcaacagaaatatataataccacgacaagttatatatgtgtgttatactgttattagaaaatatttcgtaatccatttctgatcctagccatgtataaattttgttatcttaatctagttatttcacctctacatagtacagtgctcgtgtgttgcgatgacacaatcatatttgatgagtgactctagcaatccattatcatggcatggctattatgcataaaattcacataaaagtaaattcaa

SEQ ID NO: 36 - DNA, 1975 bp, Zea mays
tccgatcctgaatttccgattggggtggcaaaggtcaagttgccaccacaagcatccagtccaatggctctgccactgcccagaagcttcatcacacctagaggtagccatgacaggacccaaaaaaaggtccagtccaggtccgtaccagctgcgacgacgcttgtcagtaggtaggttgagctagctgcttgttgatcactgctatatatacgggtgccatggatccatgccttctccatcctcaagtcatcagctagctagccttccctacagcaactgcttacatacaacacttccatcttcccgagctcgtcttcgatcaattcccaagtcaaataataatataacaacaatggtggttcccgtcatcgacttctccaagctggacggcgctgagagggccgaaaccctggcgcagatcgccaatggctgcgaggagtggggattcttccagctcgtgaaccacggcatcccgctggagcttcttgagcgcgtcaagaaggtgagctccgactgctaccgcctccgggaggccgggttcaaggcgtcggagccggtgcgcacgctggaggcgctcgtcgacgcggagcggcgcggcgaggttgtggcgccggtggatgacctggactgggaggacatcttctacatccacgacggatgccagtggccgtccgagccgccggcgttcaaggagaccatgcgcgagtaccgcgccgagctgaggaagctcgccgagcgcgtcatggaggccatggacgagaacctcggcctcgccaggggcaccatcaaggacgccttctccagcggcggccggcacgagcccttcttcggcaccaaggtcagccactacccgccgtgcccgcgcccggacctcatcacgggcctgcgcgcgcacaccgacgccggcggcgtcatcctgctgttccaggacgacagggtcggcggcctggaggtgctcaaggacggccagtggaccgacgtgcagccgctcgcgggcgccatcgtcgtcaacactggcgaccagattgaggtgctcagcaacgggcgctaccgcagcgcctggcaccgcgtgctgcccatgcgcgacggcaaccgccgctccatcgcttccttctacaacccggccaacgaggccaccatctcgccggcggcggtgcaggccagcggcggcgacgcataccccaagtacgtgttcggcgactacatggacgtgtacgccaagcacaagttccaggccaaggagcccaggttcgaagccgtcaaggttgcagcgcccaagtcatctccagcggcataaataaatggaggggaccaattattaaatgcattataatttatttgttgaataaaacagccggagaaataatgataatgtaaagtatatatgataaacaccggttaggatttaaggtgtttaactttagttgcatggtataatatgatatattgttgtagcaataagtttattaagtattcataagtgttctaaatagtgggctaaggcacttatccatcgcctttctcaaacagaaaatagtgatttaattcgggctatagcgactaatagttgctatatatattaggcgtagtagcaaacaatttcaccctttggaaacagttatatctagaaataactatagccagagatttagaaccttgttaatcatgtagaaattaaaggttcgtcaagtcagagcggcaccgaacaagataaaaatgtgacctcccctatatgcaaatgtctgccaacttattacattggtgggtgccatcttactatgtacaaatatatcgcggaaaccatattatcagcgtcgagaattggccatacccctggatattgataatatgccttgcgagatctattgagctgaagaaaactcgtagtgggtctagctagtgccatacctaaactactgggtctcgtgccctgaggagttataacatgtttctactaaatcttagggtcctc

SEQ ID NO: 37 - DNA, 1738 bp, Zea mays
cacctcctgctcgcgggccatagactgcatgcggagtgcaaatacgaagtctgctggaaacggggacagatacggagagaagagagaaactgttggccgtgctaaatacggatacggagagagagtctgctggagttggtctaagctgccaatgaaatgaacccgtagctgcctccaagaaacttctctccccgtttgccacatgctcaaacttgctgaccgtcgacctgtgtacacctggtggctggtgccctataaaacctcaaccatggcctccgaccacaaacacatgatcagctgcatgcaactaagctttcactgaagcaagcaaacaaacacctaaagatctgctatttgagtatttcttgtttctcttcagcttcatcagccatggtggttcccgtgatcgacttctccaagctggacggcgctgagaggaccgagactctggcgcagatcgccaatggctgcgaggaatggggattcttccagcttgtgaaccatggcatcccgctggagcttcttgagcgcgtcaagaaggtgtgctccgactgctaccgcctccgagaggccgggttcaaggcgtcggagccagtgcgcacgttggaggcgctcgtcgacgcggagcggcgcggcgaggaggtggcgcctgtggatgacctggactgggaggacatattcttcatccacgacggctgccagtggccgtccgacccgtcggcgttcaaggagaccatgcgcgagtaccgcgccgagctgaggaagctcgccgagcgcgtcatggaggccatggacgagaaccttggcctcaccaagggcaccatcaaggatgccttctccgccggcggccggcacgagcccttcttcggcaccaaggtcagccactacccgccgtgcccgcgcccggacctcatcacgggcctgcgcgcgcacaccgacgctggcggagtcatcctgctgttccaggatgacagagtcggtggcctggaggtgctcaaggacggccagtggatcgacgtgcagccgctcgcgggcgccatcgtcatcaacaccggcgatcagatcgaggtgctcagcaacgggcggtaccgcagcgcctggcaccgcgtgctgcccatgcgcgacggcaaccgccgctccatcgcctccttctacaacccggccaacgaggccaccatctcgccggcggcggtgcagggcagcggcggtggtgagacgtaccccaagtacgtgttcggtgattacatggacgtgtatgtcaagcagaagttccaagccaaggagcccagattcgaagccgtcaaggccgcggcgcccaagtcatctccggcggcctaaaacttgcactagacaacttctttatctagtgctaaaacgtttgcggagagttaaaatgtcgggcactctgataaagacaaaatttaccgagtattcgacaaagaactcttctccaatagtgttgccgcttaaggacacaaactcaatacaggatggtaaaattatttgggttgctattttgtttcatcgtgttgagcgtgaaaatgtaatcctaatattcttgttcctcgtgttcaatgacatatattggattattttacctcttttgtccagaaaattttatcaaagaaggccatgattataatttcttaatctaggattatcgaagtttcgaacctcgctctgacaattaatttgttgtgcgtgttccgggctccaaacggtatgcgaggtgcgcgta

SEQ ID NO: 38 - DNA, 1659 bp, Zea mays
tcctgctcgcgggccccagctgtcatagactgcatgcggagtgcaaatacggagtctgctggaaacggggacagatacggagagaagagagaaactgttggccgtgctaaatacggatacggagagggagtctgctggagttggtctaagctgccaatgaaatgaacccgtagctgcctccaagaaacttctctccccgtttgccacatgctcaaacttgctgaccgtcgacctgtgtacacctggtggctggtgccctataaaacctcaaccatggcctccgaccacaacacatgatcagctgcatgcaactaagctttcactgaagcaagcaaacaaacacctaaagatctgctatttgagtatttctcgtttctcttcagcttcatcagccatggtggttcccgtgatcgacttctccaagctggacggcgctgagaggaccgagactctggcgcagatcgccaatggctgcgaggaatggggattcttccagcttgtgaaccatggcatcccgctggagcttcttgagcgcgtcaagaaggtatgctccgactgctaccgcctccgggaggccgggttcaaggtgtcggagccagtgcgcacgttggaggcgctcgtcgacgcggagcggcgcggcgaggaggtggcgcctgtggatgacctggactgggaggacatattcttcatccacgacggctgccagtggccgtccgacccgtcggcgttcaagaagaccatacgcgagtaccgcgccgagctgaggaagctcgccgagcgcgtcatggaggccatggacgagaacctcggcctcaccaagggcaccatcaaggatgccttctccggcggcggccggcacgagcccttcttcggcaccaaggtcagccactacccgccgtgcccgcgcccggacctcatcacgggcctgcgtgcgcacaccgacgctggcggagtcatcctgctgttccaggatgacagagtcggtggcctggaggtgctcaaggacggccagtggatcgacgtgcagccgctcgcgggcgccatcgtcatcaacaccggcgatcagatcgaggtgctcagcaacgggcggtaccgcagcgcctggcaccgcgtgctgcccatgcgcgacggcaaccgccgctccattgcctccttctacaacccggctaacgaggccaccatctcgccggcggcggtgcagggcagcagcggtggtgagacgtaccccaagtacgtgttcggtgattacatggacgtgtatgtcaagcagaagttccaagccaaggagcccagattcgaagccgtcaaggccgcggcgcccaagtcatctccggcggcctaaaacttgcactagacaacttctttatctagtgctaaaacgtttgcggagagttaaatgttgggcactcgataaagacaaagtttaacgagtattggacaaagaacttttctccaatagtgttgccgcttaaggacacaaactcaatacaggatggtaaaattatttgagttgctattttgtttcatcgtgttgagcctgaaaatgtaatcctaatactcttgttcctcgtgttcaatgacatatattggattattttacctcttttgtccagaaaattttatcaaagaaggccatgattataatttcttaagctagga

SEQ ID NO: 39 - DNA, 1975 bp, Zea mays
atcagagtaccaggactgacgctacctacgccgcgtccggccggcgcgctgtcttgtccacccgggccgggaaacggaaacctgccattccaaaccaagcaacacgaaaccgcgggacgaagtttcgttgctgctgctactcactccactccagtccggtccaactgctgcagaattccacatggaatgtgggctccatccagcttcacccatttcacctgcaatgcaaggtgtgtgtttttggtgcgaattccagtataaatagccagctacccatataccttcctctcatgcagcagcgaacaacacaaattaagtagtggagtgtcagaacttgggaggcacaaattaagtacaaagcagtctaattaatgacgggcccgatggagattccggtgatcgatctcggcggcctcaacggcggcggcgaggagaggtcgcggaccttggcggagctccacgacgcctgcaaggactggggcttcttctgggtaagcagagcaccaacgaatgcttgcaattaatatttgacaacttctttccatatgcatgcgcgcgggcgtacgtacgtcattatgatgcgccggcgccgctcgcatccgcatcgcaggtggagaaccacggcgtggacgcgccgctgatggacgaggtcaagcgcttcgtctacggccactacgaggagcacctggaggccaagttctacgcctccgccctcgccatggacctcgaggccgccaccagaggtgacactgatgagaagccctccgacgaggtggactgggagtccacctacttcatccagcaccaccccaagaccaacgtcgccgacttcccagagatcacgccgccgacacggtccgtatatatactgctgtgctgccttcgtcgattcgacctcaattagttgttgccgcacacccacacaccatgcatgcttcgtacgcgctatcattcttcatcttcatgtaacacgcagagagacgctggacgcgtacgtcgcgcagatggtgtccctcgcggagcgtctggccgagtgcatgagcctcaacctgggcctccccggggcccacgtcgccgccaccttcgcgccgccgttcgtgggcaccaagttcgccatgtacccgtcctgcccgcgcccggagctggtgtggggcctgcgcgcgcacaccgacgccggcggcatcatcctgctcctccaggacgacgtcgtgggcggcctcgagttcctcagggccggcgcccactgggtccccgtcggccccaccaaggggggcaggctcttcgtcaacatcggggaccagatcgaggtcctcagcgccggcgcctaccggagcgtcctgcaccgcgtcgcggccggggaccagggccgccgcctgtccgtggccacgttctacaaccctggcaccgacgccgtggtcgcgccggcgccccgcagggatcaggacgccggcgccgcggcgtaccccggtccctacaggttcggggactacctcgactactaccagggcaccaagttcggcgacaaggacgccaggttccaggccgtcaagaagctgctcggctaagcgaacagctgcaagtaggcagaggcagcttagctcgtggactatgcatagtttcaagcttgctgcttgcttcttgttcgatccattgtctgcatgcgtactgttgcgtgtttaaatttagcaaatcttatacgtagtcgttactggtactacgtattctgtggttgacaatacattgttgcggtttaagggcgcatccgtttggtggacttgcacatgccattcgacaaaaaagttggctttccttgtcaattaatagtcaactagtacaatgacacgcaaatattcggtaagcacacatgtccacatgttgaaaaaacatctgcaagcttccgttcagtttacgtgaaaatcaaaggggttaccggatcaagaaaaaaaaatataaaactaaatatatccaacacgaa

SEQ ID NO: 40 - DNA, 2449 bp, Zea mays
cgttctcttcctgcctctaaatattgttatttattccctaataacgcgaagtcgccggccatcggcatgacacaaataaataaataaataaatatttaaaaaaggcgcatcacaagaaccaaagtaaacaccggccagaacgacaatgcatgccttggttcccttgcaaaccaatccaagctcccagtgtaaatcagtcccctgattgattggattagttgagctttcaaaataaacaattatttgacacctaacttgttcagctataaaaggctcaggggctacacagcctccaccaccatccaatatccactgcaccacttctgctaatcccttgttcttgtgcctccgatccggagctctcaccattgtcatcgtcaatcgatcaatataaagcgagccaattaccccaaggagctaccgcttgcgacggtatggcgatcccggtgattgacttctccaagctggacggccctgagagggccgagaccatggcggccctcgctgccgggttcgagcacgtggggttcttccagctggtgaacaccggcatctccgacgacctgctggagcgggtgaagaaggtgtgcagcgactcctacaagctgcgggacgaggcgttcaaggactccaaccccgcggtgaaggcgctcacagagctcgtggacaaggagatcgaggacggcctccccgcgaggaagataaaggacatggactgggaggacgtcttcaccctccatgacgacctgccatggccttccaaccctcccgccttcaagtgagagttccattccacgcatgcatgcatgattctaaattgcttccgtgctttagtttcagtttttggttaaccttttgtgctgactgctgacgcgtgtggtgcgcgcgcatgcagggagacgatgatggagtaccgcagggagctgaagaagctggcggagaagatgctgggcgtgatggaggagctgctggggttggaggagggccacatcaggaaggccttcagcaacgacggcgagttcgagcccttctacggcaccaaggtcagccactacccgccgtgcccgcggccggacctcatcgacggcctgcgcgcgcacaccgacgccggcggcctcatccttctgttccaggatgaccgcttcggcggcctgcaggcgcagcttccggacggcagctgggtcgacgtccagcccctcgagaacgccatcgtcatcaacaccggcgaccagatcgaggtacgctcatcatattcttccactactattcccttacctagcttatatatataataatatatgccgttgaataatgcatgcatgggacggtggacttcggagctcgctcgctctcctcaccttgattagattacaattgatcagtagcgagccgcttaattaatgagcctgagtgcttgcttacattgctgactgatgatgacccataaaaataatatactcctgcgtatcggtcaaacaaatcatgtcaggatttcgtttgctgtggccttgtctgattcgtcaagatccatgaattccttatgaaacatagaatgtcaaaaccttagctttgctagtttggttgttgacatgtactaccgtagtactaccttttcatgtgacttgtgactaacgagaagggattgcattgacaggtgctgagcaatggccggtacaagagcgcatggcaccgcatcctggcgacccgcgacggcaaccggcgctccatcgcctccttctacaacccagcgcgcctggccaccatcgctccggcgatccccgccgcaggggtcggcgacgacgactacccgagcttcgtgttcggcaactacatggaggtgtacgtcaagcagaagttccagcctaaggcgcccagatttgaagccatggccacgacgacgaccaagtgatgacctagcagcgactcagcgagagcctaaataaatattaattcacagtcgtcaagttaatcttgtggttatacggtacgggcggggcttgtacttatgtaggttgctaagtcttaagtgtgtagtttaattaacgtgtgtgtggaatgtacgcgtcatacaaatgtgttggtgtgtgccctgccgcaagattgcggtgagcggtggatctatggtcaacgggtgcctaaatgatttgtgcttttgtagcataaaatggcacatctcctctgcttttgttacatctccaccttttctttttgcacttttcacctcaagtaaaacatgtggcggctttcactaagtacaaagaagctctacagagctatttctattagtgtttttcagtgccgccaatgctagaccagtgaaaatcggcattttcactaacagttgttaggaactgtcattgaaaatgctattttcactagcagttttcttaaagaaactatcagtgaaaatatcatttatactagtggttggtaaagacaacagcaagtg

SEQ ID NO: 41 - DNA, 299 bp, Artificial
  construct_1
tggactgggagagcaccttcttcgtccgccacctcccggcctccaacctcgccgacctccccgacgtcgacgaccgctacaggcaggtgatggagcagttcgcatcggagatccgcaagctgtcggagaggctgctggacctgctgtgcgagaacctgggcctggagcccgggtacctgaaggcggccttcgcggggtcggacggcccgacgttcggcaccaaggtgagcgcgtacccgccgtgcccgcgcccggacctcgtcgacggcctccgcgcgcacaccgacgccggcggcatc

SEQ ID NO: 42 - DNA, 200 bp, Artificial
  construct_2
gccgccgcctgtccgtggccacgttctacaaccctggcaccgacgccgtggtcgcgccggcgccccgcagggatcaggacgccggcgccgccgcgtaccccggtccctacaggttcggggactatctagactactaccagggcaccaagttcggcgacaaggacgccaggttccaggccgtcaagaagctgctcggctaa

SEQ ID NO: 43 - DNA, 202 bp, Artificial
  construct_3
tggtggttcccgtcatcgacttctccaagctggacggcgctgagagggccgaaaccctggcgcagatcgccaatggctgcgaggagtggggattcttccagctcgtgaaccacggcatcccgctggagcttcttgagcgcgtcaagaaggtgagctccgactgctaccgcctccgggaggccgggttcaaggcgtcggagcc

SEQ ID NO: 44 - DNA, 1056 bp, Arabidopsis thaliana
atggagtcaactgatcgttcaagtcaagcaaaagctttcgacgaggccaaaatcggtgtgaaagggcttgtggattcaggaatcacagagattccggccctgttccgtgcaacgccggctactcttgcaagcctgaagtcgccaccacctccaaagcatctcaccatccctaccgttgatctcaaaggagcaagcgtggtggagaagatcggagaagctgctgagaaatggggattattccatttggtgaatcacggcatcccggtggaggttctggagaggatgattcaagggattcgcgggtttcacgagcaagaacctgaagccaagaaacgcttctactctagggatcacactagagacgtgctttactttagcaatcatgatctccaaaactccgaggccgccagttggagagacactctcggttgttataccgcacccgagcctcccagattagaggatttgcccgcggtttgcggggagattatgctggagtactcaaaggaaataatgagtttaggtgaaaggctatttgagcttctatcagaggctttggggttgaactctcatcatctcaaggacatggactgtgccaagtctcaatatatggttggccaacactacccaccttgccctcagcctgaccttactataggcataaacaagcacaccgatatttcctttctcaccgttcttcttcaagacaatgttggagggcttcaagttttccatgaacagtattggattgatgttactcctgtccctggggctctagtcattaacattggagattttcttcagcttataaccaatgataagttcataagcgcggagcatagggtgatagccaatggatcttctgaaccgcggacttccgtggcaattgttttcagcacgttcatgagggcgtattctcgagtatatgggccaatcaaagatctcctgtctgcagaaaaccctgctaagtatagagactgcaccctcaccgaattttcaaccatcttcagctcaaaaacgctcgatgctcctaagttacaccatttcaaaatctaa

SEQ ID NO: 45 - protein, 351 aa, Arabidopsis thaliana
MESTDRSSQAKAFDEAKIGVKGLVDSGITEIPALFRATPATLASLKSPPPPKHLTIPTVDLKGASVVEKIGEAAEKWGLFHLVNHGIPVEVLERMIQGIRGFHEQEPEAKKRFYSRDHTRDVLYFSNHDLQNSEAASWRDTLGCYTAPEPPRLEDLPAVCGEIMLEYSKEIMSLGERLFELLSEALGLNSHHLKDMDCAKSQYMVGQHYPPCPQPDLTIGINKHTDISFLTVLLQDNVGGLQVFHEQYWIDVTPVPGALVINIGDFLQLITNDKFISAEHRVIANGSSEPRTSVAIVFSTFMRAYSRVYGPIKDLLSAENPAKYRDCTLTEFSTIFSSKTLDAPKLHHFKI

SEQ ID NO: 46 - DNA, 963 bp, Arabidopsis thaliana
atggagaagaacatgaagtttccagtagtagacttgtccaagctcaatggggaagagagagaccaaaccatggctctaatcaatgaagcttgtgagaattggggcttctttgagatagtgaaccatggattaccacatgacttaatggacaagatcgagaagatgacaaaggaccattacaagacatgccaagaacaaaagttcaatgacatgctcaagtccaaaggtttggataatcttgagacagaagtcgaagatgtcgattgggaaagcactttctacgttcgtcacctccctcaatccaatctcaatgacatttcagatgtgtctgatgaatacaggacggccatgaaagactttggtaagagactggagaatcttgctgaggatttgttggatctactgtgtgagaatctagggttagagaaagggtatttgaagaaagtgtttcatggaacaaaaggcccaacctttgggacaaaggtgagcaattatccaccatgtcctaaaccagagatgatcaaaggtcttagggcccacactgatgcaggaggcatcatcttgttgtttcaagacgacaaggtcagtggtctccagcttcttaaagatggtgactggattgatgttcctcctctcaaccactctattgtcatcaatcttggtgaccaacttgaggtgataaccaacgggaagtataagagtgtgctgcaccgtgtggtgactcaacaagaaggaaacaggatgtcggttgcatcgttttacaacccgggaagcgatgcggagatctcaccagctacttcgcttgtcgagaaagattccgagtacccgagtttcgtctttgatgactacatgaagctttatgcaggggtcaagtttcagcccaaggagccacggttcgcagcaatgaagaatgcttctgcagttacagaactgaatcctacagcagccgtagagactttctaa

SEQ ID NO: 47 - protein, 320 aa, Arabidopsis thaliana
MEKNMKFPVVDLSKLNGEERDQTMALINEACENWGFFEIVNHGLPHDLMDKIEKMTKDHYKTCQEQKFNDMLKSKGLDNLETEVEDVDWESTFYVRHLPQSNLNDISDVSDEYRTAMKDFGKRLENLAEDLLDLLCENLGLEKGYLKKVFHGTKGPTFGTKVSNYPPCPKPEMIKGLRAHTDAGGIILLFQDDKVSGLQLLKDGDWIDVPPLNHSIVINLGDQLEVITNGKYKSVLHRVVTQQEGNRMSVASFYNPGSDAEISPATSLVEKDSEYPSFVFDDYMKLYAGVKFQPKEPRFAAMKNASAVTELNPTAAVETF

SEQ ID NO: 48 - DNA, 933 bp, Arabidopsis thaliana
atggttttgatcaaagagagagagatggagattccagttattgattttgcagagttggatggagagaagagaagcaagaccatgtcacttcttgatcatgcatgtgataagtggggattcttcatggttgataatcatggaattgataaagagttgatggagaaagtgaagaagatgattaactctcactatgaggagcatttgaaagagaagttttaccagtcagagatggtcaaggctttgagtgaaggcaaaacctcagatgcagattgggaaagcagtttcttcatctcacataaaccaacttcaaatatctgtcagatcccaaacatttcagaggaactcagcaagacgatggatgaatatgtttgtcaactgcacaagtttgcagagagactctccaagctcatgtgtgagaatcttggtcttgatcaggaagacataatgaatgccttttctggtccaaaaggtccagcttttggaacaaaagtggctaaatacccagaatgcccacgtcctgagcttatgagagggctgagagaacatacggatgctgggggaatcatattactcctgcaggatgatcaagtgcctggtcttgagttctttaaagatgggaagtgggttcctataccgccatccaagaacaataccatttttgtcaataccggtgatcaactagagatactgagtaatgggaggtacaagagtgttgttcaccgtgtaatgacagtgaagcatggaagtagactgtcgattgctacgttttacaatccggctggtgatgccataatatctccagctccaaagctcttgtatccaagtggctaccgttttcaagactacctaaagctttattcaactaccaagtttggagacaaaggccccagacttgagaccatgaagaaaatgggaaatgcggattcagcctag

SEQ ID NO: 49 - protein, 310 aa, Arabidopsis thaliana
MVLIKEREMEIPVIDFAELDGEKRSKTMSLLDHACDKWGFFMVDNHGIDKELMEKVKKMINSHYEEHLKEKFYQSEMVKALSEGKTSDADWESSFFISHKPTSNICQIPNISEELSKTMDEYVCQLHKFAERLSKLMCENLGLDQEDIMNAFSGPKGPAFGTKVAKYPECPRPELMRGLREHTDAGGIILLLQDDQVPGLEFFKDGKWVPIPPSKNNTIFVNTGDQLEILSNGRYKSVVHRVMTVKHGSRLSIATFYNPAGDAIISPAPKLLYPSGYRFQDYLKLYSTTKFGDKGPRLETMKKMGNADSA

SEQ ID NO: 50 - DNA, 1080 bp, Arabidopsis thaliana
atggcggaaaactacgaccgtgccagtgagttaaaagcattcgacgagatgaagattggcgtgaaaggactcgtcgacgccggagtcacaaaagtcccgcgcattttccataacccgcatgttaacgtagcaaaccctaagcctacatcgacggtggtgatgattccaacaatcgatctaggtggcgtgttcgaatccacggtcgtgcgagagagtgtagttgcgaaggttaaagacgcaatggagaagtttggatttttccaggcgattaaccatggggttccacttgatgtgatggagaagatgataaatggtattcgtcggtttcacgaccaagatccagaagtgaggaaaatgttctatacccgagacaaaaccaaaaagcttaaatatcactctaatgctgatctctatgagtctcctgctgcgagttggagagataccttaagttgtgtcatggctcctgatgttccaaaagcacaggacttacctgaggtttgtggggagatcatgttggagtactcaaaggaagtgatgaagttagcggagttaatgtttgaaattttatcagaagctttagggttgagtcctaaccacctcaaagaaatggattgcgcaaaaggtttatggatgctctgtcattgttttccaccctgtcctgagccaaaccgaacattcggcggcgctcagcacacagacagatctttccttactattcttcttaacgacaacaatggaggacttcaagttctctacgatggatactggatcgatgttcctcctaatcccgaagcacttatctttaacgtaggagatttcctccagcttatctcgaatgacaagtttgtaagcatggagcatagaattttggcaaatggaggtgaagagccgcgcatttcggtcgcttgtttctttgtgcatacttttacttcaccaagttcgagagtatatggacccattaaagagcttctgtctgagctaaaccctccaaaatacagagacaccacctcggaatcctccaatcactatgtggctagaaaacctaatgggaattcttcgttggaccatttaaggatctga

SEQ ID NO: 51 - protein, 359 aa, Arabidopsis thaliana
MAENYDRASELKAFDEMKIGVKGLVDAGVTKVPRIFHNPHVNVANPKPTSTVVMIPTIDLGGVFESTVVRESVVAKVKDAMEKFGFFQAINHGVPLDVMEKMINGIRRFHDQDPEVRKMFYTRDKTKKLKYHSNADLYESPAASWRDTLSCVMAPDVPKAQDLPEVCGEIMLEYSKEVMKLAELMFEILSEALGLSPNHLKEMDCAKGLWMLCHCFPPCPEPNRTFGGAQHTDRSFLTILLNDNNGGLQVLYDGYWIDVPPNPEALIFNVGDFLQLISNDKFVSMEHRILANGGEEPRISVACFFVHTFTSPSSRVYGPIKELLSELNPPKYRDTTSESSNHYVARKPNGNSSLDHLRI

SEQ ID NO: 52 - DNA, 1098 bp, Arabidopsis thaliana
atgacagaaaaatctgcagaactcgttcgtttgaacgaactcaaggcttttgtatcgacaaaagcaggtgtgaaaggacttgtcgataccaaaataaccgaagttcctcgaatcttccatatcccttcttcttcaactttatctaacaacaaaccttctgatatctttggcttaaacctcactgtcccaatcattgacctcggagatggtaacacatctgctgcaagaaacgtcctcgtttccaagattaaagaagcagctgagaattggggatttttccaagtaatcaatcatggtattcctttaactgttcttaaagatatcaaacaaggtgttcgaagatttcatgaggaagatccagaggtcaagaaacagtattttgctacagatttcaatacaagatttgcttacaacaccaacttcgatattcattattcttctcctatgaattggaaagactctttcacttgctacacttgtcctcaagatcctctaaagccagaggaaatcccactagcttgcagggatgttgtgattgaatactcgaagcatgtaatggaattaggaggtttactcttccaacttctctcagaagctttaggtttagactctgagattcttaagaacatggattgtctcaagggtttgcttatgctctgccattattatccaccttgtccacaacctgacctaactttgggcataagtaaacacaccgacaattccttcataacaattcttcttcaagatcaaatcggtggtcttcaagttcttcatcaagattcttgggttgatgtaactcctgttcctggagctcttgtcatcagtatcggtgatttcatgcagctgatcacaaacgataagttcttaagtatggagcatagggtacgggcaaacagagatggaccgcggatttcagttgcttgcttcgttagctcgggagtgtttccaaattccactgtttatggaccgataaaagagcttctttctgatgaaaaccctgcaaagtacagagacatcactataccagaatacactgtaggatacctagcaagcatcttcgatggaaaatcgcatttgtctaagttccggatatga

SEQ ID NO: 53 - protein, 365 aa, Arabidopsis thaliana
MTEKSAELVRLNELKAFVSTKAGVKGLVDTKITEVPRIFHIPSSSTLSNNKPSDIFGLNLTVPIIDLGDGNTSAARNVLVSKIKEAAENWGFFQVINHGIPLTVLKDIKQGVRRFHEEDPEVKKQYFATDFNTRFAYNTNFDIHYSSPMNWKDSFTCYTCPQDPLKPEEIPLACRDVVIEYSKHVMELGGLLFQLLSEALGLDSEILKNMDCLKGLLMLCHYYPPCPQPDLTLGISKHTDNSFITILLQDQIGGLQVLHQDSWVDVTPVPGALVISIGDFMQLITNDKFLSMEHRVRANRDGPRISVACFVSSGVFPNSTVYGPIKELLSDENPAKYRDITIPEYTVGYLASIFDGKSHLSKFRI

SEQ ID NO: 54 - DNA, 873 bp, Arabidopsis thaliana
atgacagaaaaatctgcagaactcgttcgtttgaacgaactcaaggcttttgtatcgacaaaagcaggtgtgaaaggacttgtcgataccaaaataaccgaagttcctcgaatcttccatatcccttcttcttcaactttatctaacaacaaaccttctgatatctttggcttaaacctcactgtcccaatcattgacctcggagatggtaacacatctgctgcaagaaacgtcctcgtttccaagattaaagaagcagctgagaattggggatttttccaagtaatcaatcatggtattcctttaactgttcttaaagatatcaaacaaggtgttcgaagatttcatgaggaagatccagaggtcaagaaacagtattttgctacagatttcaatacaagatttgcttacaacaccaacttcgatattcattattcttctcctatgaattggaaagactctttcacttgctacacttgtcctcaagatcctctaaagccagaggaaatcccactagcttgcagggatgttgtgattgaatactcgaagcatgtaatggaattaggaggtttactcttccaacttctctcagaagctttaggtttagactctgagattcttaagaacatggattgtctcaagggtttgcttatgctctgccattattatccaccttgtccacaacctgacctaactttgggcataagtaaacacaccgacaattccttcataacaattcttcttcaagatcaaatcggtggtcttcaagttcttcatcaagattcttgggttgatgtaactcctgttcctggagctcttgtcatcagtatcggtgatttcatgcaggcaagctcgattgatgcttccttttaa

SEQ ID NO: 55 - protein, 290 aa, Arabidopsis thaliana
MTEKSAELVRLNELKAFVSTKAGVKGLVDTKITEVPRIFHIPSSSTLSNNKPSDIFGLNLTVPIIDLGDGNTSAARNVLVSKIKEAAENWGFFQVINHGIPLTVLKDIKQGVRRFHEEDPEVKKQYFATDFNTRFAYNTNFDIHYSSPMNWKDSFTCYTCPQDPLKPEEIPLACRDVVIEYSKHVMELGGLLFQLLSEALGLDSEILKNMDCLKGLLMLCHYYPPCPQPDLTLGISKHTDNSFITILLQDQIGGLQVLHQDSWVDVTPVPGALVISIGDFMQASSIDASF

SEQ ID NO: 56 - DNA, 1089 bp, Arabidopsis thaliana
atgacagagaattctgaaaaaatcgatcgtttaaacgatctcacgacttttatctcgacgaagacaggagtgaaaggactcgtcgatgccgaaataaccgaagttcctagcatgtttcatgtcccttcttctattttatcaaacaacagaccttctgatatctccggcttaaacctcaccgtcccaatcatcgacctcggagatcgtaacacatcttcaagaaacgttgtcatttcgaagatcaaagacgcagctgagaattggggatttttccaagtgatcaatcatgatgttcctttaactgttcttgaagagatcaaagagagtgttcgaaggtttcatgaacaagatccagttgtcaagaaccaatatcttcctaccgataacaacaagagatttgtttataacaatgatttcgatctctatcattcttctcctttgaattggagagactctttcacttgttatattgctccagatcctccgaatccagaggaaatcccactagcttgcaggagtgcggtgatcgaatacacgaagcatgtaatggaattaggagctgtgctcttccaacttctctcagaagctttaggtttagactctgagacacttaagaggattgattgtcttaagggtttgtttatgctctgccattactatccaccttgcccacaacctgacctaactttaggtataagtaaacacaccgacaactctttcctcacgcttcttcttcaagaccaaatcggtggtcttcaagttcttcatgaagattattgggtcgatgtccctcctgtacctggagctcttgttgtcaacattggtgatttcatgcagctgataacgaacgataagttcttgagcgtggagcatagggtacgaccgaacaaagatagaccgcggatttcagttgcgtgcttctttagctcgagtctttctccaaattccacggtttatggaccgattaaagatcttttgtctgatgaaaaccctgctaagtacaaagatatcaccataccagagtacactgcaggatttcttgcgagcatttttgatgaaaagtcgtatttgactaattacatgatatga

SEQ ID NO: 57 - protein, 362 aa, Arabidopsis thaliana
MTENSEKIDRLNDLTTFISTKTGVKGLVDAEITEVPSMFHVPSSILSNNRPSDISGLNLTVPIIDLGDRNTSSRNVVISKIKDAAENWGFFQVINHDVPLTVLEEIKESVRRFHEQDPVVKNQYLPTDNNKRFVYNNDFDLYHSSPLNWRDSFTCYIAPDPPNPEEIPLACRSAVIEYTKHVMELGAVLFQLLSEALGLDSETLKRIDCLKGLFMLCHYYPPCPQPDLTLGISKHTDNSFLTLLLQDQIGGLQVLHEDYWVDVPPVPGALVVNIGDFMQLITNDKFLSVEHRVRPNKDRPRISVACFFSSSLSPNSTVYGPIKDLLSDENPAKYKDITIPEYTAGFLASIFDEKSYLTNYMI

SEQ ID NO: 58 - DNA, 966 bp, Oryza sativa
atggcgagtgttgcctccttcccggtgatcaacatggagaacctggagaccgaggagaggggcgcagcaatggaggtcatccgcgacgcctgcgagaactggggcttcttcgagatgctgaaccatggcatcgcgcacgagctgatggacgaggtggagcgggtgagcaaggcgcactacgccaactgccgggaggagaagttcaaggagttcgcgcggcggatgctggaggccggcgagaagggcgccgacgtgaagggcatcgactgggagagcaccttcttcgtccgccaccgccccgtctccaacctcgccgacctccccgacgtcgacgaccactacaggcaggtgatgaagcaatttgcgtcggagatcgagaagctctcggagagggtgctggacctgctgtgcgagaatctgggcctggagaagggttacctgaagaaggccttcgccgggtcgaacggcccaacgttcggcaccaaggtgagcagctacccgccgtgcccgcgccccgatctcgtcgacggcctccgcgcccacaccgacgccggtggcatcatcctgctgttccaggacgaccaggtgagcggcctccagctgctcaaggacggggagtgggtggacgtgccgcccatgcgccacgccatcgtcgccaacatcggcgaccagctggaggtgatcaccaacggcaggtacaagagcgtcatgcaccgcgtcctcacgcgccccgacggcaaccgcatgtccatcgcctccttctacaaccccggcgccgacgccgtcatcttcccggcgcccgcgctcgccgccgccgacgcggcggcggccgcctacccgaggttcgtgttcgaggactacatgaacctgtacgtgcgccacaagttcgaggccaaggagccacgcttcgaggccatgaagtccgccgccgaggtcgtccacgcggcgcccatcgccaccgcttga

SEQ ID NO: 59 - protein, 321 aa, Oryza sativa
MASVASFPVINMENLETEERGAAMEVIRDACENWGFFEMLNHGIAHELMDEVERVSKAHYANCREEKFKEFARRMLEAGEKGADVKGIDWESTFFVRHRPVSNLADLPDVDDHYRQVMKQFASEIEKLSERVLDLLCENLGLEKGYLKKAFAGSNGPTFGTKVSSYPPCPRPDLVDGLRAHTDAGGIILLFQDDQVSGLQLLKDGEWVDVPPMRHAIVANIGDQLEVITNGRYKSVMHRVLTRPDGNRMSIASFYNPGADAVIFPAPALAAADAAAAAYPRFVFEDYMNLYVRHKFEAKEPRFEAMKSAAEVVHAAPIATA

SEQ ID NO: 60 - DNA, 969 bp, Oryza sativa
atggcggcagcattgtcgttcccgatcatcgacatgagtctgctcgacggggcagagaggcccgcggcgatggggctgctccgcgacgcatgcgagagctggggcttctttgagatcctgaaccacggcatctcgacggagctgatggacgaggtggagaagatgaccaaggaccactacaagcgtgtgcgcgagcagaggttcctcgagttcgcgagcaagacgctcaaggaaggctgcgacgacgtgaataaggcggagaagctggactgggagagcaccttcttcgtccgccacctcccggagtccaacatcgccgacatacccgacctcgacgacgactacaggcgcctcatgaagcgcttcgcggcggagctggagacgctggcggagcggctactggacctgctctgcgagaacctcggcctcgagaagggctacctcaccaaggccttccgtggccccgcgggcgcacccaccttcggcaccaaggtcagcagctacccgccgtgcccgcgccccgacctcgtcaagggcctccgcgcccacaccgacgccggcggcatcatcctgctcttccaggacgaccgcgtcggtggcctccagctgctcaaggacggcgagtgggtggacgtgccgcccatgcgccactccatcgtcgtcaacctcggcgaccagctggaggtgatcaccaacggcaggtacaagagcgtgatgcaccgggtggtggcgcagatcgacggcaacaggatgtccatcgcgtccttctacaaccctggcagcgacgccgtcatctccccggcgccggcgctggtgaaggaggaggaggccggcgagacgtatcccaagttcgtgttcgaggactacatgaagctgtacgtgcgccacaagttcgaggccaaggagccccggttcgaggcgttcaaggccatggagaacgagacccccaaccgcattgccatcgcttga

SEQ ID NO: 61 - protein, 322 aa, Oryza sativa
MAAALSFPIIDMSLLDGAERPAAMGLLRDACESWGFFEILNHGISTELMDEVEKMTKDHYKRVREQRFLEFASKTLKEGCDDVNKAEKLDWESTFFVRHLPESNIADIPDLDDDYRRLMKRFAAELETLAERLLDLLCENLGLEKGYLTKAFRGPAGAPTFGTKVSSYPPCPRPDLVKGLRAHTDAGGIILLFQDDRVGGLQLLKDGEWVDVPPMRHSIVVNLGDQLEVITNGRYKSVMHRVVAQIDGNRMSIASFYNPGSDAVISPAPALVKEEEAGETYPKFVFEDYMKLYVRHKFEAKEPRFEAFKAMENETPNRIAIA

SEQ ID NO: 62 - DNA, 969 bp, Oryza sativa
atggcaccgacttcgacgttcccggtcatcaacatggagttgctcgccggggaggagcgacctgcggcgatggagcagctggatgatgcttgcgagaactggggattcttcgagatcctgaaccacggcatctcgacggagctgatggacgaggtggagaagatgaccaaggaccactacaagcgtgtgcgcgagcagaggttcctcgagttcgcgagcaagacgctcaaggaaggctgcgacgacgtgaataaggcggagaagctggactgggagagcaccttcttcgtccgccacctcccggagtccaacatcgccgacatacccgacctcgacgacgactacaggcgcctcatgaagcgcttcgcggcggagctggagacgctggcggagcggctactggacctgctctgcgagaacctcggcctcgagaagggctacctcaccaaggccttccgtggccccgcgggcgcacccaccttcggcaccaaggtcagcagctacccgccgtgcccgcgccccgacctcgtcgagggcctccgcgcccacaccgacgccggcggcatcatcctgctcttccaggacgaccgcgtcggtggcctccagctgctcaaggacggcgagtgggtggacgtgccgcccatgcgccactccatcgtcgtcaacctcggcgaccagctggaggtgatcaccaacggcaggtacaagagcgtgatccaccgggtggtggcgcagaccgacggcaacaggatgtccatcgcgtcgttctacaaccctggcagcgacgccgtgatctcccctgcgccggcgctggtgaaggaggaggaggccgtcgtggcgtaccccaagttcgtgttcgaggactacatgaagctgtacgtgcgccacaagttcgaggccaaggagcccaggttcgaggcgttcaagtccatggaaaccgagacctccaaccgcatcgccatcgcttag

SEQ ID NO: 63 - protein, 322 aa, Oryza sativa
MAPTSTFPVINMELLAGEERPAAMEQLDDACENWGFFEILNHGISTELMDEVEKMTKDHYKRVREQRFLEFASKTLKEGCDDVNKAEKLDWESTFFVRHLPESNIADIPDLDDDYRRLMKRFAAELETLAERLLDLLCENLGLEKGYLTKAFRGPAGAPTFGTKVSSYPPCPRPDLVEGLRAHTDAGGIILLFQDDRVGGLQLLKDGEWVDVPPMRHSIVVNLGDQLEVITNGRYKSVIHRVVAQTDGNRMSIASFYNPGSDAVISPAPALVKEEEAVVAYPKFVFEDYMKLYVRHKFEAKEPRFEAFKSMETETSNRIAIA

SEQ ID NO: 64 - DNA, 939 bp, Oryza sativa
atggagattccagtgattgatctcaaggggctcgccggcggcgacgaagaaagggagcgcaccatggcccagctccacgaggcctgtaaggactggggcttcttctgggtggaaaaccatggcgtggaggcggcgttaatggaggaggtgaagagcttcgtgtaccgccattacgacgagcacctggagaagaaattctacgcctccgacctcgccaagaacctccacctgaacaaggacgacggcgacgtcctcgtcgacggcggcgacctcgccgaccaggccgactgggaggccacctacttcatccagcaccgccccaagaacaccgccgccgacttcccggacatcccgccggcggcgagggagtccctggacgcgtacatcgcgcaggcggtgtccctcgccgagctgctcgccggctgcatcagcaccaacctgggcctcgccggcgccgccggcgtcgtggacgccttcgcgccgccgttcgtcggcaccaagttcgccatgtacccaccgtgcccgcgcccggacctcgtctggggcctccgcgcccacaccgacgccggcggcatcatcctgctcctccaggacgacgccgtcggcgggctcgagttccaccgcggcggccgcgagtgggtccccgtcggcccgacccggcgcggccggctgttcgtcaacatcggcgaccaggtggaggtgctcagcggcggcgcctacaagagcgtcgtgcaccgcgtcgccgccggcgccgagggccgccgcctgtccgtcgccacgttctacaaccccgggcccgacgccgtgatcgcgccggcgacggcggcggcgccgtaccccgggccgtacaggtacggcgactacctggactactaccagggcaccaagttcggcgacaagaccgctaggttccaggccgtcaagaagctcttcagctga

SEQ ID NO: 65 - protein, 312 aa, Oryza sativa
MEIPVIDLKGLAGGDEERERTMAQLHEACKDWGFFWVENHGVEAALMEEVKSFVYRHYDEHLEKKFYASDLAKNLHLNKDDGDVLVDGGDLADQADWEATYFIQHRPKNTAADFPDIPPAARESLDAYIAQAVSLAELLAGCISTNLGLAGAAGVVDAFAPPFVGTKFAMYPPCPRPDLVWGLRAHTDAGGIILLLQDDAVGGLEFHRGGREWVPVGPTRRGRLFVNIGDQVEVLSGGAYKSVVHRVAAGAEGRRLSVATFYNPGPDAVIAPATAAAPYPGPYRYGDYLDYYQGTKFGDKTARFQAVKKLFS

SEQ ID NO: 66 - DNA, 930 bp, Oryza sativa
atggcgatcccggtcatcgacttctccaagctcgacggcgatgagagcgaggccaccctggcggagctcgctgcggggtttgaggagtgggggttcttccagctggtgaacactggcatccctgatgatctgctggaaagggtgaagaaggtgtgcagtgacatctacaagctgcgcgaggatgggttcaaagaatccaaccccgcagtgaaggctctcgcccgcctggtagaccaggaaggcgagggcctcgcaatgaagaaaatcgaggacatggactgggaggacgtcttcaccctccaggacgacctgccctggccctccaaccctccatccttcaaggagacgatgatggagtacaggagggagctgaagaagctggcagagaagctgctgggagtgatggaggagcttcttggtctggaggaagggcacatcaggaaggccttcaccaacgacggcgacttcgagcccttctacggcaccaaggtgagccactacccgccgtgcccgcggccggagctcgtcgacggcctccgcgcccacaccgacgccggcggcctcatcctcctcttccaggacgaccgcttcggcggcctccagatgatccccaaccgcggcggcgacggccggtggatcgacgtccagcccgtcgagaacgccatcgtcgtcaacaccggggaccagatcgaggtgcttagcaatggccgcttcaagagcgcatggcacagaatcctggccacccgggacggcaatcgccggagcatcgcctccttctacaacccggcgcgcatggccaacattgctccggcgatccccgccgccgccgccgactacccgagcttcaagttcggcgactacatggaggtgtacgtgaagcagaagttccaggccaaggagcccaggttcgcagccctggcgaacaagtga

SEQ ID NO: 67 - protein, 309 aa, Oryza sativa
MAIPVIDFSKLDGDESEATLAELAAGFEEWGFFQLVNTGIPDDLLERVKKVCSDIYKLREDGFKESNPAVKALARLVDQEGEGLAMKKIEDMDWEDVFTLQDDLPWPSNPPSFKETMMEYRRELKKLAEKLLGVMEELLGLEEGHIRKAFTNDGDFEPFYGTKVSHYPPCPRPELVDGLRAHTDAGGLILLFQDDRFGGLQMIPNRGGDGRWIDVQPVENAIVVNTGDQIEVLSNGRFKSAWHRILATRDGNRRSIASFYNPARMANIAPAIPAAAADYPSFKFGDYMEVYVKQKFQAKEPRFAALANK

SEQ ID NO: 68 - DNA, 927 bp, Oryza sativa
atggttgttccggtgatcgacttctccaagctcgacggcaccgccgcagagagggctgagacgatggcgcagatcgacaatggctgcgaggagtggggattcttccagctggtgaaccatggcgtcccgaaggagcttcttgatcgggtgaagaaggtgtgcttggagagctaccgactccgggaggcggcgttcatggagtcggagccggtgaggacgctggaggggctcatggcggcggagcggcgcggcgaggcggcggcgccggtggacgacatggactgggaggacatcttctacctccacgacgacaaccagtggccgtcgaacccgccggagttcaaggagacgatgcgcgagtaccgcgcggcgctgcgggggctcgccgagagggtgatggaggccatggacgagaacctcggcctcgacaaggggcgcatgaggcgcgccttcaccggcgacggccgccacgcgccgttcttcggcaccaaggtcagccactacccgccgtgcccgcgccccgacctcatcaccggcctccgcgcccacaccgacgccggcggcgtcatcctgctgttccaggacgaccgcgtcggcggcctccaggtgctcaggggcggcgagtgggtcgacgtgcagccgctcgccgacgccatcgtcgtcaacaccggcgaccaggtggaggtgctcagcaacggccgctaccgcagcgcgtggcaccgcgtcctccccatgcgcgacggaaaccggcgctccgtcgcgtcgttctacaacccggcgttcgaggccaccatctcgccggcggtgggcgccggcggcgagtacccggagtacgtgttcggcgagtacatggatgtgtacgccaagcagaagttcgatgcgaaggagccacgcttcgaggccgtcaaggcgccaaaatctgcttaa

SEQ ID NO: 69 - protein, 308 aa, Oryza sativa
MVVPVIDFSKLDGTAAERAETMAQIDNGCEEWGFFQLVNHGVPKELLDRVKKVCLESYRLREAAFMESEPVRTLEGLMAAERRGEAAAPVDDMDWEDIFYLHDDNQWPSNPPEFKETMREYRAALRGLAERVMEAMDENLGLDKGRMRRAFTGDGRHAPFFGTKVSHYPPCPRPDLITGLRAHTDAGGVILLFQDDRVGGLQVLRGGEWVDVQPLADAIVVNTGDQVEVLSNGRYRSAWHRVLPMRDGNRRSVASFYNPAFEATISPAVGAGGEYPEYVFGEYMDVYAKQKFDAKEPRFEAVKAPKSA

SEQ ID NO: 70 - DNA, 690 bp, Oryza sativa
atggtggttccggtgatcaacttctccaagctcgacggcaccgccgcggagagggccgagacgatggcgcagatcgacaatggctgcgaggagtggggattcttccagctggtgaaccatggcgtcccgaaggagcttcttgatcgggtgaagaagctaccgactccgggaggcggcgttcatggagtcgagccggtgaggacgctggaggggctcatggcggcggagcggcgcggcgaggcggcggcgccggtggacgacatggactgggaggacatcttctacctccacgacgacaaccagtggccgtcgaaaccgccggagttcaaggagacgatgcgggagtaccgcgcggcgctgcgggggctcgccgagagggtgatggaggccatggacgagaacctcggcctcgacaaggggcgcatgaggcgcgccttcaccggcgacggccgccacgcgccgttcttcggcaccaaggtcagccactacccgccgtgcccgcgccccgacctcatcaccggcctccgcgcccacaccgacgccggcggcgtcatcctgctgttccaggacgaccgcgtcggcggcctccaggtgctcaggggcggcgagtgggtcgacgtgcagccgctcgccgacgccatcgtcgtcaacaccggcaaccaggtggaggtgctcagcaacggccgctaa

SEQ ID NO: 71 - protein, 229 aa, Oryza sativa
MVVPVINFSKLDGTAAERAETMAQIDNGCEEWGFFQLVNHGVPKELLDRVKKLPTPGGGVHGVEPVRTLEGLMAAERRGEAAAPVDDMDWEDIFYLHDDNQWPSKPPEFKETMREYRAALRGLAERVMEAMDENLGLDKGRMRRAFTGDGRHAPFFGTKVSHYPPCPRPDLITGLRAHTDAGGVILLFQDDRVGGLQVLRGGEWVDVQPLADAIVVNTGNQVEVLSNGR